SEQUENCE LISTING (53 sequences)

SEQ ID NO: 1 - DNA, 60 bp, synthetic construct
  source 1..60: mol_type = other DNA
  modified_base 50: mod_base = OTHER; note = thymidine-succinyl hexamide CED phosphoramidite
  modified_base 51: mod_base = OTHER; note = thymidine-succinyl hexamide CED phosphoramidite
agacaatcaaccatttggggtggacagccttgacctctagacttcggcattttttttttt

SEQ ID NO: 2 - DNA, 110 bp, synthetic construct
  source 1..110: mol_type = other DNA
  modified_base 50: mod_base = OTHER; note = thymidine-succinyl hexamide CED phosphoramidite
  modified_base 51: mod_base = OTHER; note = thymidine-succinyl hexamide CED phosphoramidite
cgggatccttatcgtcatcgtcgtacagatcccgacccatttgctgtccaccagtcatgctagccataccatgatgatgatgatgatgagaaccccgcattttttttttt

SEQ ID NO: 3 - DNA, 19 bp, synthetic construct
  source 1..19: mol_type = other DNA
atgcggggttctcatcatc

SEQ ID NO: 4 - DNA, 20 bp, synthetic construct
  source 1..20: mol_type = other DNA
cgggatccttatcgtcatcg

SEQ ID NO: 5 - DNA, 22 bp, synthetic construct
  source 1..22: mol_type = other DNA
cagaacgttgttttcaggaagt

SEQ ID NO: 6 - DNA, 13 bp, synthetic construct
  source 1..13: mol_type = other DNA
aatacacaaagat

SEQ ID NO: 7 - DNA, 13 bp, synthetic construct
  source 1..13: mol_type = other DNA
catacgtgatggc

SEQ ID NO: 8 - DNA, 10 bp, synthetic construct
  source 1..10: mol_type = other DNA
aggctcacca

SEQ ID NO: 9 - DNA, 10 bp, synthetic construct
  source 1..10: mol_type = other DNA
aggcaaccgt

SEQ ID NO: 10 - DNA, 16 bp, synthetic construct
  source 1..16: mol_type = other DNA
gcccagatggacatga

SEQ ID NO: 11 - DNA, 16 bp, synthetic construct
  source 1..16: mol_type = other DNA
ggaattaagagaagca

SEQ ID NO: 12 - DNA, 10 bp, synthetic construct
  source 1..10: mol_type = other DNA
tatgggattg

SEQ ID NO: 13 - DNA, 16 bp, synthetic construct
  source 1..16: mol_type = other DNA
ttgggagatcttgcac

SEQ ID NO: 14 - DNA, 25 bp, synthetic construct
  source 1..25: mol_type = other DNA
gcagacctctaggcaggcccaggcc

SEQ ID NO: 15 - DNA, 10 bp, synthetic construct
  source 1..10: mol_type = other DNA
tgtggccgag

SEQ ID NO: 16 - DNA, 28 bp, synthetic construct
  source 1..28: mol_type = other DNA
actgcttccctgaggaggagaagtgctt

SEQ ID NO: 17 - DNA, 13 bp, synthetic construct
  source 1..13: mol_type = other DNA
gagatgtttatga

SEQ ID NO: 18 - DNA, 17 bp, synthetic construct
  source 1..17: mol_type = other DNA
tgctttcaccctcagcg

SEQ ID NO: 19 - DNA, 17 bp, synthetic construct
  source 1..17: mol_type = other DNA
gaagctgtacttcacaa

SEQ ID NO: 20 - DNA, 20 bp, synthetic construct
  source 1..20: mol_type = other DNA
catgagtctagtacttaatg

SEQ ID NO: 21 - DNA, 23 bp, synthetic construct
  source 1..23: mol_type = other DNA
ctgtcttctgggattatcagaac

SEQ ID NO: 22 - DNA, 18 bp, synthetic construct
  source 1..18: mol_type = other DNA
gtaaaagtttagtaagta

SEQ ID NO: 23 - DNA, 15 bp, synthetic construct
  source 1..15: mol_type = other DNA
ttgattctagcacgc

SEQ ID NO: 24 - DNA, 18 bp, synthetic construct
  source 1..18: mol_type = other DNA
actacacaaatattgagg

SEQ ID NO: 25 - DNA, 26 bp, synthetic construct
  source 1..26: mol_type = other DNA
tgaaggactaaaggatcttcgaagac

SEQ ID NO: 26 - DNA, 23 bp, synthetic construct
  source 1..23: mol_type = other DNA
taagaaaagtatggatgatcaag

SEQ ID NO: 27 - DNA, 17 bp, synthetic construct
  source 1..17: mol_type = other DNA
aaatctggtgactatac

SEQ ID NO: 28 - DNA, 19 bp, synthetic construct
  source 1..19: mol_type = other DNA
actgtccccattggtgaat

SEQ ID NO: 29 - DNA, 17 bp, synthetic construct
  source 1..17: mol_type = other DNA
tgagacagttcctttta

SEQ ID NO: 30 - DNA, 20 bp, synthetic construct
  source 1..20: mol_type = other DNA
tgctccgccacctctaccag

SEQ ID NO: 31 - DNA, 30 bp, synthetic construct
  source 1..30: mol_type = other DNA
gatgaggagggcgcatgccagccttgcccc

SEQ ID NO: 32 - DNA, 29 bp, synthetic construct
  source 1..29: mol_type = other DNA
tctccactggcaccctccgaaggggctgg

SEQ ID NO: 33 - DNA, 28 bp, synthetic construct
  source 1..28: mol_type = other DNA
agatcccgactccgggacagcaccagcc

SEQ ID NO: 34 - DNA, 13 bp, synthetic construct
  source 1..13: mol_type = other DNA
caccccctccgaa

SEQ ID NO: 35 - DNA, 17 bp, synthetic construct
  source 1..17: mol_type = other DNA
ccttggcgaatccacca

SEQ ID NO: 36 - DNA, 31 bp, synthetic construct
  source 1..31: mol_type = other DNA
agaagatttgctgaaccctattggtgttact

SEQ ID NO: 37 - DNA, 14 bp, synthetic construct
  source 1..14: mol_type = other DNA
ctaaaatggcagtg

SEQ ID NO: 38 - DNA, 13 bp, synthetic construct
  source 1..13: mol_type = other DNA
tctgacagagtta

SEQ ID NO: 39 - DNA, 19 bp, synthetic construct
  source 1..19: mol_type = other DNA
gctgaaaaacaatctgact

SEQ ID NO: 40 - DNA, 17 bp, synthetic construct
  source 1..17: mol_type = other DNA
aatgaaactttcatttg

SEQ ID NO: 41 - DNA, 12 bp, synthetic construct
  source 1..12: mol_type = other DNA
cattgcagaatt

SEQ ID NO: 42 - DNA, 22 bp, synthetic construct
  source 1..22: mol_type = other DNA
attcactttgtcttagaggagt

SEQ ID NO: 43 - DNA, 25 bp, synthetic construct
  source 1..25: mol_type = other DNA
caatacaagcgactatagaggaaaa

SEQ ID NO: 44 - DNA, 16 bp, synthetic construct
  source 1..16: mol_type = other DNA
atttccagtcctgcag

SEQ ID NO: 45 - DNA, 26 bp, synthetic construct
  source 1..26: mol_type = other DNA
ctagagttctccttggaaatgagagc

SEQ ID NO: 46 - DNA, 13 bp, synthetic construct
  source 1..13: mol_type = other DNA
gacatgtccccca

SEQ ID NO: 47 - DNA, 16 bp, synthetic construct
  source 1..16: mol_type = other DNA
catgtgaacgctactt

SEQ ID NO: 48 - DNA, 14 bp, synthetic construct
  source 1..14: mol_type = other DNA
ataaccgaggagtt

SEQ ID NO: 49 - DNA, 14 bp, synthetic construct
  source 1..14: mol_type = other DNA
taattgtgcatgag

SEQ ID NO: 50 - DNA, 23 bp, synthetic construct
  source 1..23: mol_type = other DNA
ggcgtcccgcttgtccctgcgga

SEQ ID NO: 51 - DNA, 12 bp, synthetic construct
  source 1..12: mol_type = other DNA
gcatacgtgatg

SEQ ID NO: 52 - DNA, 20 bp, synthetic construct
  source 1..20: mol_type = other DNA
aatgatacggcgaccaccga

SEQ ID NO: 53 - DNA, 24 bp, synthetic construct
  source 1..24: mol_type = other DNA
caagcagaagacggcatacgagat

(19) United States
(12) Patent Application Publication
(10) Pub. No.: US 2026/0275331 A1
(43) Pub. Date: Sep. 17, 2026

(54) VARIANT-CAPTURE MINIMAL RESIDUAL DISEASE PANELS

(71) Applicant: Twist Bioscience Corporation, South San Francisco, CA (US)

(72) Inventors: Derek MURPHY, South San Francisco, CA (US); Michael BOCEK, Inver Grove Heights, MN (US)

(73) Assignee: Twist Bioscience Corporation, South San Francisco, CA (US)

(21) Appl. No.: 19/474,594

(22) PCT Filed: Apr. 12, 2024
(86) PCT No.: PCT/US2024/024409

§ 371 Date: Oct. 10, 2025

Related U.S. Application Data
(60) Provisional application No. 63/495,938, filed on Apr. 13, 2023.

(51) Int. Cl. C12N 15/10

(52) U.S. Cl. CPC C12N 15/1093

(57) ABSTRACT

Described herein are compositions for detecting genomic variants associated with minimal residual disease (MRD). The compositions include libraries comprising a plurality of polynucleotides comprising at least one variant associated with minimal residual disease (MRD). Further described herein are methods of preparing such polynucleotide libraries, and methods of detecting MRD in a sample using such polynucleotide libraries.

### CROSS-REFERENCE TO RELATED APPLICATIONS

This application claims priority to U.S. Provisional Patent Application No. 63/495,938, filed Apr. 13, 2023, the entirety of which is incorporated herein by reference. All publications, patents, and patent applications mentioned in this specification are herein incorporated by reference to the same extent as if each individual publication, patent, or patent application was specifically and individually indicated to be incorporated by reference.

### BACKGROUND

Identification of genomic variants with high fidelity and low cost has a central role in biotechnology and medicine, and in basic biomedical research. While various methods are known for identification of genomic variants in complex nucleic acid samples, these techniques often suffer from scalability, automation, speed, sensitivity, accuracy, and cost.

### SUMMARY

Provided herein are polynucleotide libraries. In some aspects, the polynucleotide library comprises a plurality of polynucleotides. In some aspects, each polynucleotide of the plurality of polynucleotides comprises a nucleic acid sequence having a center. In some aspects, the nucleic acid sequence of each polynucleotide comprises at least one variant sequence associated with minimal residual disease (MRD).

In some embodiments, the location of the at least one MRD-associated variant sequence is within 20 bases of the center of each nucleic acid sequence of each polynucleotide. In some embodiments, the polynucleotide library further comprises a distribution of locations of the at least one MRD-associated variant sequence in the nucleic acid sequences of all polynucleotides of the plurality of polynucleotides, wherein the distribution comprises a mean within 20 bases of the center of each nucleic acid sequence of each polynucleotide. In some embodiments, the nucleic acid sequence of each polynucleotide is no more than 150 bases in length. In some embodiments, the at least one MRD-associated variant sequence is derived from genomic sequences. In some aspects, the genomic sequences are derived from cell-free DNA (cfDNA). In some embodiments, the at least one MRD-associated variant sequence is present in the plurality of polynucleotides at a frequency of 0.001% to 0.1% relative to a wild-type genomic sequence. In some embodiments, the plurality of polynucleotides comprises about 500 variant sequences associated with MRD. In some embodiments, the nucleic acid sequence of each polynucleotide comprises a variant sequence of the at least one MRD-associated variant sequence. In some embodiments, the at least one MRD-associated variant is present in nucleic acid sequences of at least 150 genes. In some embodiments, the at least one MRD-associated variant sequence comprises a modification relative to a nucleic acid sequence of a tumor suppressor gene or an oncogene. In some embodiments, the polynucleotide library further comprises a background set of polynucleotides, wherein the at least one MRD-associated variant sequence is at least one base pair different than a nucleic acid sequence of a polynucleotide of the background set. In some aspects, the background set comprises cell-free DNA (cfDNA).

Also provided herein are methods of preparing a polynucleotide library comprising a plurality of polynucleotides. In some aspects, the method comprises: providing at least one variant sequence associated with minimal residual disease (MRD); and synthesizing a plurality of polynucleotides comprising the at least one MRD-associated variant sequence to produce the polynucleotide library.

In some embodiments, the method further comprises: providing a background set of polynucleotides; and mixing the background set and the plurality of polynucleotides such that the at least one MRD-associated variant sequence is present at a frequency of no more than 2% relative to a wild-type genomic sequence. In some embodiments, synthesizing comprises chemical synthesis, synthesis on a surface, or coupling of nucleoside phosphoramidites. In some embodiments, the method further comprises: sequencing the polynucleotide library.

Also provided herein are methods of detecting minimal residual disease (MRD) in a sample. In some aspects, the method comprises: providing a polynucleotide library as described herein; contacting the polynucleotide library with a sample; and detecting a presence or an absence of the at least one variant associated with MRD in the sample.

### BRIEF DESCRIPTION OF THE DRAWINGS

FIGS. 1A-1B are schematics illustrating probe designs, according to aspects of the present disclosure. FIG. 1A illustrates a probe design in which the probes do not contain mutations, whereas

FIG. 1B illustrates a probe design in which the probes themselves incorporate the variant sequences.

FIG. 2 is a graph of estimated allele fractions for different variants, according to aspects of the present disclosure. The variants include single-based substitution (SBS), small indels, large indel (>5 base pairs), and structural variants (SV), and the estimated allele fraction is shown from 0% to 14% in increments of 2% for alignments and K-mer searches.

FIG. 3 is a schematic illustrating an experimental design to test probes, according to aspects of the present disclosure.

FIG. 4 is a schematic illustrating a bioinformatics workflow for analyzing variant detection using probes, according to aspects of the present disclosure.

FIG. 5 is a panel of three bar graphs depicting target variant allele frequencies and measured variant allele frequencies for probes with (“Ref+Alt) and without (“Ref only”) variant sequences, according to aspects of the present disclosure. Measured variant allele frequency (VAF) is measured as a function of target VAF for single-based substitution (SBS) sites, indel sites, and all sites.

FIG. 6 is a panel of three bar graphs depicting target variant allele frequencies and recall rates with (“Ref+Alt”) and without (“Ref only”) variant sequences, according to aspects of the present disclosure. Recall is measured as a function of target VAF for single-based substitution (SBS) sites, indel sites, and all sites.

FIG. 7 is a panel of point graphs related to probe panel performance with the following Picard metrics: (A) Off target rate; (B) Fold80 uniformity; (C) Zero coverage target rate; and (D) Mean target coverage. The probe panels include breast, CRC, kidney, lung, and melanoma.

FIG. 8 is a panel of five bar graphs depicting mean positive variant allele frequency (VAF) detected at different targeted VAF levels for each of the probe panels of FIG. 7, according to aspects of the present disclosure.

FIG. 9 is a panel of five point graphs depicting variant recall rates at different variant allele frequency (VAF) levels for each of the probe panels of FIG. 7, according to aspects of the present disclosure.

FIG. 10 is a panel of five scatter plots depicting mean recall rates for variant allele frequencies (VAF) of different variants, according to aspects of the present disclosure. The variants include single-based substitutions (SBS), single base indels, small indels, medium indels, and large indels.

FIG. 11 is a panel of five scatter plots depicting recalls per sample for variant allele frequencies (VAF) of different variants, according to aspects of the present disclosures. The variants include small indels (2-4 base pairs), medium indels (5-9 base pairs), and large indels (10+ base pairs).

FIG. 12 is a panel of five scatter plots depicting mean recall rates for variant allele frequencies (VAF) of different variants, according to aspects of the present disclosure. The variants include single-based substitutions (SBS), single base indels, small indels, medium indels, and large indels.

FIGS. 13A-13B are panels of Receiver Operating Characteristic (ROC) curve analyses to show the connection between sensitivity and specificity with different target sites at each variant allele frequency (VAF) level, according to aspects of the present disclosure. FIG. 13A is a panel of ten ROC curve analyses for 0.01% VAF and 0.05% VAF at 10, 20, 50, 100, and 197 sites. FIG. 13B is a panel of ten ROC curve analyses for 0.10% and 2.00% VAF at 10, 20, 50, 100, and 197 sites.

FIG. 14 is a panel of four scatter plots depicting ultra-low variant allele frequency (VAF) results for number of positive sites and measured VAF on linear and log scales, according to aspects of the present disclosure.

FIG. 15 is a schematic illustrating a plate having 256 clusters, each cluster having 121 loci with polynucleotides extending therefrom, according to aspects of the present disclosure.

FIG. 16 is a schematic illustrating a computer system, according to aspects of the present disclosure.

FIG. 17 a block diagram illustrating an architecture of a computer system, according to aspects of the present disclosure.

FIG. 18 is a diagram illustrating a network configured to incorporate a plurality of computer systems, a plurality of cell phones and personal data assistants, and Network Attached Storage (NAS), according to aspects of the present disclosure.

FIG. 19 is a block diagram illustrating a multiprocessor computer system using a shared virtual address memory space, according to aspects of the present disclosure.

FIG. 20A is a frequency plot of polynucleotide representation across a plate from synthesis of 29,040 unique polynucleotides from 240 clusters, each cluster having 121 polynucleotides, according to aspects of the present disclosure. Polynucleotide frequency is shown as a function of abundance as measured via absorbance units.

FIG. 20B is a series of frequency plots of polynucleotide representation across each individual cluster (with control clusters identified by boxes), according to aspects of the present disclosure. Polynucleotide frequency is shown as a function of abundance as measured via absorbance units.

FIG. 21A is a schematic illustrating a workflow for attachment of adapters comprising unique molecular identifiers (UMIs) to a polynucleotide to form an adapter-ligated polynucleotide, according to aspects of the present disclosure.

FIG. 21B is a schematic illustrating a workflow for amplification of adapter-ligated polynucleotides to form a library for sequencing, according to aspects of the present disclosure.

FIG. 21C is a schematic illustrating a workflow for synthesis of a polynucleotide adapter comprising a unique molecular identifier (UMI), according to aspects of the present disclosure.

FIG. 21D is a schematic illustrating a workflow for synthesis of a polynucleotide adapter comprising a unique molecular identifier (UMI), wherein the method comprises PCR extension of one strand of the adapter, according to aspects of the present disclosure.

FIG. 21E is a schematic illustrating a workflow for synthesis of a polynucleotide adapter comprising a unique molecular identifier (UMI), wherein the method comprises PCR extension of one strand of the adapter followed by restriction enzyme cleavage, according to aspects of the present disclosure.

FIG. 21F is a schematic illustrating a workflow for synthesis of a polynucleotide adapter comprising a unique molecular identifier (UMI), wherein the method comprises restriction enzyme cleavage, according to aspects of the present disclosure.

FIG. 22 is a schematic illustrating a workflow for duplex sequencing analysis to identify variants, according to aspects of the present disclosure. An asterisk (“*”) indicates potential errors introduced by PCR or sequencing, and a plus sign (“+”) indicates true variants.

FIG. 23A is a graph depicting a design of synthetic circulating tumor DNA (ctDNA) to target a variant site, according to aspects of the present disclosure. Multiple overlapping or “tiled” polynucleotides are configured to contain the variant site (indicated with a star). Labeled oligonucleotides (y-axis) is shown as a function of labeled genome coordinate from 0-300 at 100 unit intervals (x-axis).

FIG. 23B is a frequency graph depicting a distribution of indel sizes for a synthetic circulating tumor (ctDNA) library, including short, medium (5-10 base pairs), and large (~30 base pairs) size variants, according to aspects of the present disclosure. Positive numbers indicate insertions, and negative numbers indicate deletions. Labeled number of variants from 0-40 at 20 unit intervals (y-axis) is shown as a function of labeled indel size from −30-10 base pairs at 10 unit intervals.

FIG. 23C is a line graph depicting abundance versus size for background cell-free DNA (cfDNA), according to aspects of the present disclosure. Abundance was measured via signal in fluorescence units (FU), and background cfDNA was obtained from healthy donor plasma. Abundance from 0-400 fluorescence units at 50 unit intervals (y-axis) is shown as a function of labeled base pairs (bp) at 35, 100, 150, 200, 300, 400, 500, 600, 1000, 2000, and 10380 base pairs. Peak 1 and peak 2 are labeled.

### DETAILED DESCRIPTION

Minimal residual disease (MRD), also known as molecular residual disease, refers to a small number of tumor cells which may remain within a patient after therapeutic intervention. The detection of these remnants and monitoring of their abundance is a promising prognostic marker to identify individuals at risk of recurrence or in need of adjuvant therapy. Due to the low abundance of circulating tumor DNA (ctDNA) present in samples obtained during remission, MRD assays need to be highly sensitive. In addition, each individual will have a different set of somatic variants, requiring personalized solutions for detection. What is needed are personalized NGS assays with high sensitivity and specificity for MRD diagnostics.

Provided herein are compositions and methods, including panels and kits, that can be used to address this need and empower accurate assessments of MRD. In some instances, such compositions and methods can enable users to design and/or manufacture fully personalized MRD panels. In some examples, the panels can comprise up to 100, 200, 300, 400 or 500 targets. In some examples, the design and/or manufacturing of panels can be performed in as little as six days.

Provided herein are polynucleotide libraries comprising at least one variant sequence. In some instances, the at least one variant sequence is associated with a minimal residual disease (MRD). In some instances, the at least one variant sequence is present at a frequency of 0.001% to 0.1% relative to a wild-type genomic sequence. In some instances, the at least one variant sequence is within 20 bases of a center of a sequence in each of the plurality of polynucleotides. In some instances, the at least one variant sequence is within 10% of a center of a sequence in each of the plurality of polynucleotides. In some instances, locations of each the at least one variant sequence in each sequence of the plurality of polynucleotides comprises a distribution comprising a mean. In some examples, the mean is a center of each sequence. In some instances, the mean is within 20 bases of the center of each sequence. In some instances, the mean is within 10% of the center of each sequence. In some instances, the plurality of polynucleotides are no more than 150 bases in length. In some instances, the plurality of polynucleotides are double-stranded.

Further provided herein are kits for detecting MRD. In some instances, the kit detects MRD in a sample, such as a biological sample from a patient and/or a user. In some instances, the kit comprises a polynucleotide library comprising at least one variant sequence, as described herein. In some instances, the kit further comprises instructions for use of the kit and/or packaging configured to hold and describe the kit contents.

Also provided herein are methods of preparing a polynucleotide library comprising at least one variant sequence as described herein. The library can be used to detect MRD. In some instances, the method comprises providing the at least one variant sequence associated with MRD. In some instances, the method comprises synthesizing the plurality of polynucleotides comprising the at least one variant sequence. In some instances, the method further comprises providing a background set of background polynucleotides. In some instances, the method further comprises mixing the background set and the plurality of polynucleotides comprising the at least one variant sequence. In some instances, mixing the background set and the plurality of polynucleotides comprises mixing the background set and the plurality of polynucleotides such that the at least one variant sequence present at a frequency of 0%, 0.01%, 0.05%, 0.1%, 0.25%, 0.5%, 1%, or 2% relative to a wild-type genomic sequence.

Further provided herein are methods of detecting MRD. In some instances, MRD may be detected in a sample, such as a biological sample from a patient and/or a user. In some instances, the method comprises providing a polynucleotide library comprising at least one variant sequence, as described herein. In some instances, the method comprises contacting the polynucleotide library with the sample. In some instances, the method comprises detecting a presence or an absence of the at least one variant sequence associated with MRD in the sample.

### Definitions

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein, the terms “preselected sequence”, “predefined sequence” or “predetermined sequence” are used interchangeably. The terms mean that the sequence of the polymer is known and chosen before synthesis or assembly of the polymer. In particular, various aspects of the invention are described herein primarily with regard to the preparation of nucleic acids molecules, the sequence of the polynucleotide being known and chosen before the synthesis or assembly of the nucleic acid molecules.

As used herein, the term “nucleic acid” encompasses double-stranded or triple-stranded nucleic acid molecules, as well as single-stranded nucleic acid molecules. In double-stranded or triple-stranded nucleic acid molecules, the nucleic acid strands need not be coextensive (i.e., a double-stranded nucleic acid need not be double-stranded along the entire length of both strands). Nucleic acid sequences, when provided, are listed in the 5′ to 3′ direction, unless stated otherwise. Methods described herein provide for the generation of isolated nucleic acid molecules. Methods described herein additionally provide for the generation of isolated nucleic acids and purified nucleic acids. The length of nucleic acid molecules (e.g., polynucleotides), when provided, are described as the number of bases and abbreviated, such as nucleotides (nt), bases or base pairs (bp), kilobases (kb), megabases (Mb) or gigabases (Gb).

As used herein, the terms “polynucleotide,” “oligonucleic acid,” “oligonucleotide,” “oligo,” and “nucleic acid molecule” are used interchangeably. Libraries of synthetic (i.e., de novo synthesized or chemically synthesized) polynucleotides described herein may comprise a plurality of polynucleotides collectively encoding for one or more genes or gene fragments. In some instances, the polynucleotide library comprises coding or non-coding nucleic acid sequences. In some instances, the polynucleotide library encodes for a plurality of cDNA sequences. Reference gene sequences from which the cDNA sequences are based may contain introns, whereas cDNA sequences exclude introns. Polynucleotides described herein may encode for genes or gene fragments from an organism. Exemplary organisms include, without limitation, prokaryotes (e.g., bacteria) and eukaryotes (e.g., mice, rabbits, humans, and non-human primates). In some instances, the polynucleotide library comprises one or more polynucleotides, each of the one or more polynucleotides encoding sequences for multiple exons. Each polynucleotide within a library described herein may encode a different nucleic acid sequence, i.e., non-identical nucleic acid sequence. In some instances, each polynucleotide within a library described herein comprises at least one portion that is complementary to the nucleic acid sequence of another polynucleotide within the library. Polynucleotide sequences described herein may, unless stated otherwise, comprise DNA or RNA. A polynucleotide library described herein may comprise at least 10, 20, 50, 100, 200, 500, 1000, 2000, 5000, 10000, 20000, 30000, 50000, 100000, 200000, 500000, 1000000, or more than 1000000 polynucleotides. A polynucleotide library described herein may have no more than 10, 20, 50, 100, 200, 500, 1000, 2000, 5000, 10000, 20000, 30000, 50000, 100000, 200000, 500000, or no more than 1000000 polynucleotides. A polynucleotide library described herein may comprise 10 to 500, 20 to 1000, 50 to 2000, 100 to 5000, 500 to 10000, 1000 to 5000, 10000 to 50000, 100000 to 500000, or 50000 to 1000000 polynucleotides. A polynucleotide library described herein may comprise about 370000, 400000, 500000, or more different polynucleotides.

### Libraries of Variants

Provided herein are polynucleotide libraries configured to detect or measure one or more variant sequences. In some instances, these libraries are used as references or controls. Known methods of generating such libraries may comprise isolating nucleic acids from biological sources (blood, plasma, cells, or patients) with an established disease or condition. However, such methods in some instances provide libraries which contain contamination from their biological source. In some instances, libraries are produced from biological samples to mimic cell-free DNA (cfDNA) by restriction digestion, sonication, or other method of generating short nucleic acid fragments. These methods may not mimic the natural fragmentation profile of cfDNA. Additionally, variant sequences in low abundance may not be detected from biologically-derived libraries. Provided herein are methods comprising design and de novo synthesis of polynucleotide libraries (or sample sets) which are useful for detecting or measuring frequencies of variant sequences. In some instances, such libraries provide enhanced accuracy for diagnosing diseases or conditions and are substantially free of biological contamination. In some instances, synthetic polynucleotide libraries provide additional control over library content, reliability/reproducibility, lack of reliance on fragmentation methods, and/or provide other advantages over traditional cell-derived libraries. In some instances, such libraries are mixed with control nucleic acid molecules (e.g., cfDNA) to generate a reference standard at a specific variant allele frequency (VAF).

In some embodiments, the polynucleotide library comprises a plurality of polynucleotides (e.g., a sample set) derived from genomic sequences. In some instances, the plurality of polynucleotides may comprise at least one variant sequence associated with a disease or condition. In some instances, the at least one variant sequence comprises one or more changes compared to a wild-type genomic sequence or a background polynucleotide. In some embodiments, the polynucleotide library comprises a background set comprising background polynucleotides, wherein the background set comprises cell-free DNA (cfDNA). In some instances, at least some of the polynucleotides are tiled across each of the at least one variant sequence. In some instances, the polynucleotides are not tiled across each of the at least one variant. In some instances, background cfDNA is obtained, derived, or expanded from a cell line or patient sample.

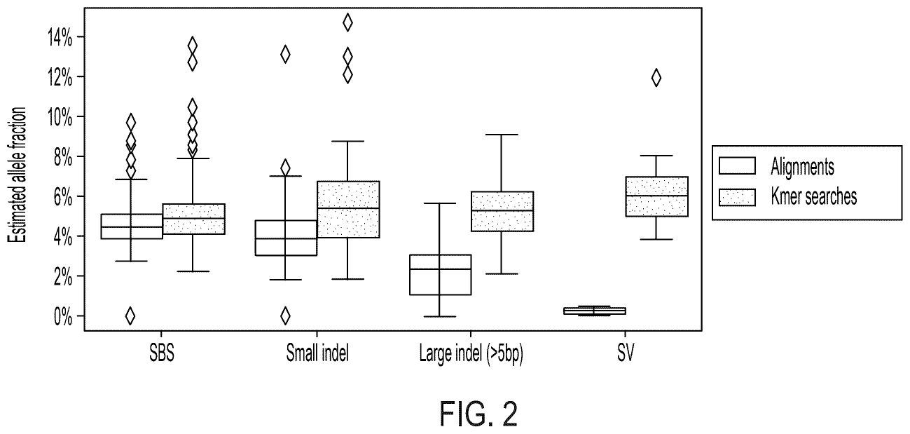

Variant allele frequencies generally observed may be lower than expected. This may be due to one or more biases, such as for example, capture bias or alignment bias. This observation is generally illustrated in FIG. 2. Generally, libraries (e.g., panels) target towards reference alleles, so an alternate allele will have mismatches to the probe, resulting in capture bias. In some examples, alignment algorithms (e.g., BWA) find reads more challenging to align if they contain large differences from the reference allele (e.g., in the context of sequencing errors), resulting in alignment bias. Both capture bias and alignment bias may favor the reference allele over the alternate, and tend to be more severe for larger-edit distances (e.g., single nucleotide polymorphisms (SNPs)<Short indels <Long indels <Structural Variants (SVs)).

In some embodiments, when sequences of genomes with variant sequences (genomic variants) are known, they can be incorporated into polynucleotides. For example, if a sequence of a cancer genome is already known, it can be incorporated to avoid probe mismatches or sequence mismatches. In some instances, the cancer comprises MRD. In some instances, the libraries do not just target the sites of variants, but also incorporate variant sequences into the probes themselves. In some instances, this design can be used to avoid reference allele bias as described herein.

Provided herein are libraries of polynucleotides comprising pre-determined variant sequences (e.g., variants). In some instances, the polynucleotide library comprises at least 1, 5, 10, 15, 20, 25, 50, 75, 100, 150, 200, 250, 300, 350, 400, 450, 500, 750, 1000, or at least 2000 variants. In some instances, the polynucleotide library comprises about 1, 5, 10, 15, 20, 25, 50, 75, 100, 150, 200, 250, 300, 350, 400, 450, 500, 750, 1000, or about 2000 variants. In some instances, the polynucleotide library comprises no more than 1, 5, 10, 15, 20, 25, 50, 75, 100, 150, 200, 250, 300, 350, 400, 450, 500, 750, 1000, or no more than 2000 variants. In some instances, the polynucleotide library comprises 1-500, 5-500, 10-500, 10-2000, 10-150, 15-500, 20-1000, 50-500, 50-750, 50-1000, 100-1000, 100-500, 100-750, 250-800, 400-1000, or 400-2000 variants. In some instances, the polynucleotide library is designed to include about 100, 150, or 200 targets, with about 1, 2, 3, 4, 5 or 6 variants per tissue origin.

Polynucleotides provided herein may be tiled across a nucleic acid region. In some instances, tiling describes the design of polynucleotides (or complements or reverse complements thereof) which cover or span a target area (such as a variant). In some instances, tiling results in increases in sensitivity for detection either for probes targeting the variant, or in the design of corresponding standards, controls, or references. This may be beneficial for regions of low abundance or regions that comprise sequences which are difficult to sequence (repeating, high/low GC, or other challenges). In some instances, each tiled polynucleotide for a target region is different from each other tiled polynucleotide for the target region. In some instances, such a tiling design comprises about 2, 3, 4, 5, 6, 7, 8, 9, 10, 11, 12, 15, 20, 25, 27, 30, 32, 35, 40, 45, or about 50 polynucleotides tiled across a region (e.g., variant). In some instances, such a tiling design comprises at least 2, 3, 4, 5, 6, 7, 8, 9, 10, 11, 12, 15, 20, 25, 30, 35, 40, 45, or at least 50 polynucleotides tiled across a region. In some instances, such a tiling design comprises 10-100, 5-50, 2-50, 25-50, 30-40, or 30-60 polynucleotides tiled across a region. In some instances, tiled polynucleotides comprise at least one overlap region with another polynucleotide. In some instances, both 5′ and 3′ termini of a tiled polynucleotide overlap with an adjacent tiled polynucleotide. In some instances, one or more tiled polynucleotides are tiled with an offset value, such that a first polynucleotide starts at a different position than the next tiled polynucleotide. In some instances, the offset is about 1, 2, 3, 4, 5, 6, 7, 8, 9, 10, 12, 15, 17, 20, 25, or 30 bases. In some instances, the offset is 1-30, 1-20, 1-10, 1-8, or 2-5 bases. In some instances, the length of at least some of the polynucleotides is 20-500, 50-500, 75-500, 100-200, 100-500, 200-500, 100-250, 100-200, 100-1000, 250-500, or 250-1000. In some instances, the length of at least some of the polynucleotides is about 50, 75, 100, 125, 150, 155, 160, 165, 170, 175, 180, 190, 200, or 225 bases. In some instances, the length of at least 80% of the polynucleotides is 20-500, 50-500, 75-500, 100-200, 100-500, 200-500, 100-250, 100-200, 100-1000, 250-500, or 250-1000. In some instances, the length of at least 80% of the polynucleotides is about 50, 75, 100, 125, 150, 155, 160, 165, 170, 175, 180, 190, 200, or 225 bases. In some instances, the length of at least 90% of the polynucleotides is 20-500, 50-500, 75-500, 100-200, 100-500, 200-500, 100-250, 100-200, 100-1000, 250-500, or 250-1000. In some instances, the length of at least 90% of the polynucleotides is about 50, 75, 100, 125, 150, 155, 160, 165, 170, 175, 180, 190, 200, or 225 bases. In some instances, at least some of the polynucleotides are double-stranded. In some instances, at least 50%, 60%, 70%, 75%, 80%, 90%, 95%, or at least 98% of the polynucleotides are double-stranded. In some instances, a plurality of polynucleotides comprising at least one variant sequence associated with low variant frequency alleles (e.g., MRD) may be no more than about 150 bases, 170 bases, or 200 bases in length.

Variant sequences may be present at a predetermined frequency relative to other variant sequences in a library (e.g., sample library). In some instances, at least 80% of the at least one variant sequences are present at a frequency that differs by no more than 20%, 15%, 12%, 10%, 8% or no more than 5% relative to the expected frequency for uniformly pooled variants. In some instances, at least 90% of the at least one variant sequences are present at frequencies that differ by no more than 20%, 15%, 12%, 10%, 8% or no more than 5% relative to the expected frequency for uniformly pooled variants. In some instances, at least 95% of the at least one variant sequences are present at frequencies that differ by no more than 20%, 15%, 12%, 10%, 8% or no more than 5% relative to the expected frequency for uniformly pooled variants. In some instances, at least 99% of the at least one variant sequences are present at frequencies that differ by no more than 20%, 15%, 12%, 10%, 8% or no more than 5% relative to the expected frequency for uniformly pooled variants.

Compositions (libraries) described herein may comprise a plurality of polynucleotides comprising at least one variant sequence associated with a minimal residual disease (MRD). In some instances, the at least one variant sequence is within 2, 5, 10, 15, 20, 25, 30, 35, 40, 45, or 50 bases of a center of a sequence in each of the plurality of polynucleotides. The center of the sequence may generally comprise a position in the sequence that lies maximally far from ends of the sequence. In some instances, the at least one variant sequence is within at least 2, 5, 10, 15, 20, 25, 30, 35, 40, 45, or 50 bases of a center of a sequence in each of the plurality of polynucleotides. In some instances, the at least one variant sequence is within at most 2, 5, 10, 15, 20, 25, 30, 35, 40, 45, or 50 bases from a center of a sequence in each of the plurality of polynucleotides. In some instances, the at least one variant sequence is within 2 to 5, 2 to 10, 2 to 15, 2 to 20, 2 to 25, 2 to 30, 2 to 35, 2 to 40, 2 to 45, 2 to 50, 5 to 10, 5 to 15, 5 to 20, 5 to 25, 5 to 30, 5 to 35, 5 to 40, 5 to 45, 5 to 50, 10 to 15, 10 to 20, 10 to 25, 10 to 30, 10 to 35, 10 to 40, 10 to 45, 10 to 50, 15 to 20, 15 to 25, 15 to 30, 15 to 35, 15 to 40, 15 to 45, 15 to 50, 20 to 25, 20 to 30, 20 to 35, 20 to 40, 20 to 45, 20 to 50, 25 to 30, 25 to 35, 25 to 40, 25 to 45, 25 to 50, 30 to 35, 30 to 40, 30 to 45, 30 to 50, 35 to 40, 35 to 45, 35 to 50, 40 to 45, 40 to 50, or 45 to 50 bases of a center of a sequence in each of the plurality of polynucleotides. In some instances, the at least one variant sequence is within 2%, 5%, 10%, 15%, 20%, 25%, 30%, 40%, or 50% of a center of a sequence in each of the plurality of polynucleotides. In some instances, the at least one variant sequence is within at least 2%, 5%, 10%, 15%, 20%, 25%, 30%, 40%, or 50% of a center of a sequence in each of the plurality of polynucleotides. In some instances, the at least one variant sequence is within at most 2%, 5%, 10%, 15%, 20%, 25%, 30%, 40%, or 50% of a center of a sequence in each of the plurality of polynucleotides. In some instances, the at least one variant sequence is within 2% to 5%, 2% to 10%, 2% to 15%, 2% to 20%, 2% to 25%, 2% to 30%, 2% to 40%, 2% to 50%, 5% to 10%, 5% to 15%, 5% to 20%, 5% to 25%, 5% to 30%, 5% to 40%, 5% to 50%, 10% to 15%, 10% to 20%, 10% to 25%, 10% to 30%, 10% to 40%, 10% to 50%, 15% to 20%, 15% to 25%, 15% to 30%, 15% to 40%, 15% to 50%, 20% to 25%, 20% to 30%, 20% to 40%, 20% to 50%, 25% to 30%, 25% to 40%, 25% to 50%, 30% to 40%, 30% to 50%, or 40% to 50% of a center of a sequence in each of the plurality of polynucleotides. In some instances, locations of each the at least one variant sequence in each sequence of the plurality of polynucleotides comprises a distribution comprising a mean. In some instances, the mean is a center of each sequence. In some instances, the mean is within 2, 5, 10, 15, 20, 25, 30, 35, 40, 45, or 50 bases of the center of each sequence. In some instances, the mean is within at least 2, 5, 10, 15, 20, 25, 30, 35, 40, 45, or 50 bases of the center of each sequence. In some instances, the mean is within at most 2, 5, 10, 15, 20, 25, 30, 35, 40, 45, or 50 bases of the center of each sequence. In some instances, the mean is within 2 to 5, 2 to 10, 2 to 15, 2 to 20, 2 to 25, 2 to 30, 2 to 35, 2 to 40, 2 to 45, 2 to 50, 5 to 10, 5 to 15, 5 to 20, 5 to 25, 5 to 30, 5 to 35, 5 to 40, 5 to 45, 5 to 50, 10 to 15, 10 to 20, 10 to 25, 10 to 30, 10 to 35, 10 to 40, 10 to 45, 10 to 50, 15 to 20, 15 to 25, 15 to 30, 15 to 35, 15 to 40, 15 to 45, 15 to 50, 20 to 25, 20 to 30, 20 to 35, 20 to 40, 20 to 45, 20 to 50, 25 to 30, 25 to 35, 25 to 40, 25 to 45, 25 to 50, 30 to 35, 30 to 40, 30 to 45, 30 to 50, 35 to 40, 35 to 45, 35 to 50, 40 to 45, 40 to 50, or 45 to 50 bases of the center of each sequence. In some instances, the mean is within 2%, 5%, 10%, 15%, 20%, 25%, 30%, 40%, or 50% of the center of each sequence. In some instances, the mean is within at least 2%, 5%, 10%, 15%, 20%, 25%, 30%, 40%, or 50% of the center of each sequence. In some instances, the mean is within at most 2%, 5%, 10%, 15%, 20%, 25%, 30%, 40%, or 50% of the center of each sequence. In some instances, the mean is within 2% to 5%, 2% to 10%, 2% to 15%, 2% to 20%, 2% to 25%, 2% to 30%, 2% to 40%, 2% to 50%, 5% to 10%, 5% to 15%, 5% to 20%, 5% to 25%, 5% to 30%, 5% to 40%, 5% to 50%, 10% to 15%, 10% to 20%, 10% to 25%, 10% to 30%, 10% to 40%, 10% to 50%, 15% to 20%, 15% to 25%, 15% to 30%, 15% to 40%, 15% to 50%, 20% to 25%, 20% to 30%, 20% to 40%, 20% to 50%, 25% to 30%, 25% to 40%, 25% to 50%, 30% to 40%, 30% to 50%, or 40% to 50% of the center of each sequence. In some instances, the distribution is a normal distribution.

Compositions described herein may comprise a background set (or library) of polynucleotides. In some instances, the background set mimics background cfDNA that would be present in a patient sample. In some instances, background polynucleotides are mixed with sample polynucleotides (e.g., polynucleotides comprising variant sequences, variant polynucleotide libraries) to generate reference standards or controls. In some instances, the standards or control comprises variant sequences having a VAF of 0%, 0.001%, 0.005%, 0.01%, 0.05%, 0.1% 0.25%, 0.5%, 1%, 2%, 5%, 10%, 15%, or 20% relative to a wild-type genomic sequence. In some instances, the background polynucleotide set comprises wild-type regions corresponding to locations of the at least one variant sequence. In some instances, wild-type sequences are derived from a reference database or sample. In some instances, the background polynucleotide set comprises wild-type regions corresponding to locations of the at least 1, 2, 5, 10, 15, 20, 25, 50, 75, 100, 125, 150, 200, 250, 300, 350, 400, 450, 500, or at least 500 variants. In some instances, the wild-type regions are represented within 30%, 25%, 20%, 15%, 12%, 10%, 9%, 8%, 7%, or within 5% of the variant frequency of the variant set (e.g., sample set). In some instances, the background set comprises a low level amount of variations. In some instances, at least one background polynucleotide comprises a variant present at a frequency of 0.001%, 0.005%, 0.01%, 0.05%, 0.1% 0.25%, 0.5%, 1%, or 2% relative to a wild-type genomic sequence. In some instances, at least 1% of the background polynucleotides comprise a variant sequence present at a frequency of 0.001%, 0.005%, 0.01%, 0.05%, 0.1% 0.25%, 0.5%, 1%, or 2% relative to a wild-type genomic sequence. In some instances, a background set is synthesized from pre-determined sequences. In some instances, the pre-determined sequences reflect desired variant frequencies. In some instances, synthetic background sets are used to calibrate instruments or methods by providing control over variant frequencies. In some instances, synthetic background sets are configured to mimic variant frequencies corresponding to specific samples or disease states.

In some instances, a background set comprises background polynucleotides. In some instances, a background set comprises background polynucleotides which substantially consist of wild-type sequences. In some instances, background sets are derived or isolated from a healthy individual. In some instances, the healthy individual is male. In some instances, the healthy individual is female. In some instances, the healthy individual is no more than 40, 35, 30, 25, 20, or 15 years old. In some instances, background sets are obtained from a biological sample. In some instances, the biological sample comprises blood, plasma, or another source of nucleic acids. In some instances, the background set comprises cfDNA. In some instances, the background set comprises at least 2, 5, 10, 100, 200, 500, 1000, 10,000, 100,000, 500,000 polynucleotides, 1 million, 5 million, 10 million, 50 million, 100 million, 200 million, or more than 500 million polynucleotides. In some instances, the polynucleotides of highest abundance in the background set are 100-500, 50-500, 75-250, 50-750, 50-300, 100-300, 100-200, 125-300, 150-175, 150-185, or 125-200 bases in length. In some instances, at least 50%, 60%, 70%, 75%, 80%, 85%, 90%, 95%, or at least 97% of the polynucleotides in the background set are mononucleosomal or dinucleosomal. In some instances, the ratio of mononucleosomal polynucleotides to dinucleosomal polynucleotides is 50:50 to 90:10, 60:40 to 90:10, 60:40 to 95:5, 70:30 to 95:5, 70:30 to 90:10, or 80:20 to 95:5.

Polynucleotide libraries described herein may be mixed to form standards (references). In some instances, the standard (reference) comprises both a sample (variant) polynucleotide set and a control polynucleotide set. In some instances, the standard comprising both a sample polynucleotide set and a control polynucleotide set further comprises a liquid buffer. In some instances, the buffer comprises TE or TBE buffer. In some instances, the standard comprises no more than 50%, 40%, 30%, 25%, 20%, 15%, or no more than 10% sample polynucleotides relative to background polynucleotides. In some instances, the standard comprises variant sequences having a VAF of 0%, 0.1% 0.25%, 0.5%, 1%, or 2% relative to a wild-type genomic sequence. In some instances, the standard is subjected to one or more quality control operations including one or more of fluorescence/UV DNA quantification, electrophoretic size analysis, sequencing, ddPCR analysis, or other analysis technique. In some instances, the sample polynucleotide set is subjected to one or more quality control operations including one or more of fluorescence/UV DNA quantification, electrophoretic size analysis, sequencing, ddPCR analysis, or other analysis technique prior to mixing with a background polynucleotide set. In some instances, the sample polynucleotides are ligated to adapters comprising unique molecular identifiers (UMIs) as described herein.

Provided herein are methods of preparing polynucleotide libraries as described herein. In some instances, the polynucleotide library may be used to detect variant sequences having low variant allele frequencies. In some instances, the polynucleotide library may be used to detect MRD. In some instances, the method comprises providing at least one variant sequence. In some instances, the at least one variant sequence may be associated with MRD. In some instances, the method further comprises synthesizing a plurality of polynucleotides comprising the at least one variant sequence. In some instances, the method further comprises providing a background set as described herein. In some instances, the method further comprises mixing the background set and the plurality of polynucleotides comprising the at least one variant sequence. In some instances, mixing the background set and the plurality of polynucleotides comprises mixing the background set and the plurality of polynucleotides such that the at least one variant sequence is present at a frequency of 0%, 0.01%, 0.05%, 0.1%, 0.25%, 0.5%, 1%, or 2% relative to a wild-type genomic sequence. In some instances, synthesizing comprises chemical synthesis. In some instances, synthesizing comprises synthesis on a surface. In some instances, synthesizing comprises coupling of nucleoside phosphoramidites. In some instances, the method further comprises sequencing the polynucleotide library. In some instances, the method further comprises ddPCR measurement of the polynucleotide library. In some instances, the method further comprises fluorescence/UV DNA quantification and size distribution of the polynucleotide library.

Synthetic libraries (e.g., sample libraries, sample sets, variant sets) comprising variant sequences may have fewer contaminants (less contamination) than libraries derived from biological samples. In some instances, a lower level of contaminants results in improved performance as a reference standard. In some instances, contamination includes but is not limited to cellular components, lipids, RNA, proteins, or other biomolecules derived from the biological source. In some instances, the biological source comprises plasma, cells, blood, or other source of nucleic acids. In some instances, synthetic libraries are prepared or stored in a buffer. In some instances, a synthetic library is at least 95%, 96%, 97%, 98%, 99%, 99.5%, or at least 99.7% free from biological contaminants.

### Genomic Variants

Genetic variants (“variants” in nucleic acid sequences) among populations of individuals may provide information regarding risk for diseases, identification of individuals, response to drug treatments, or susceptibility to environmental factors such as toxins. Described herein are compositions and methods involving synthesis of polynucleotide libraries which contain such variant sequences. In some instances, variant sequences comprise a single nucleotide polymorphism (SNP), a single nucleotide variation (SNV), an indel, a copy number variation, a translocation, fusion, inversion, or structural variant. In some instances, an SNP differs between individuals in the same population. In some instance, an SNP differs between individuals in different populations. In some instances, an SNV comprises a variation in a single nucleotide without any limitations of frequency. In some instances, polynucleotide libraries (e.g., probe libraries) described herein are used to identify such variants after sequencing. In some instances, polynucleotide libraries are configured to enrich for nucleic acid molecules (e.g., fragments of a genome) which comprise variant sequences. In some instances, such nucleic acid molecules are captured using the polynucleotide libraries and sequenced for variant calling. In some instances, variant calls may be assessed compared to known variant sequences using metrics such as recall and/or precision for one or all of the variant sequences. In some instances, an SNP or SNV is heterozygous. In some instances, an SNP or SNV is homozygous. In some instances, an SNP or SNV is homozygous in matching a reference sequence. In some instances, a variant sequence is homozygous for a state other than that observed in the human reference genome. In some instances, a variant sequence is identified after sequencing by comparison to a reference database. In some instances, the reference database comprises GiAB, dbSNP, DoGSD, dbGaP, clinvar, ncbi, refseq, refSNP, COSMIC, or any other database which comprises known variants. In some instances, the variant sequence comprises an insertion, deletion, fusion, duplication, frameshift, repeat expansion, or substitution. In some instances, the variant sequence comprises a copy number variant (CNV), microsatellite instability, loss of heterozygosity (LOH), DNA methylation, premature stop codon, trinucleotide repeat, translocation, somatic rearrangement, allelomorph, single nucleotide variant (SNV), indel, splice variant, regulator variant, copy number variant, or fusion. In some instances, the indels are 1-50, 1-25, 1-20, 1-15, 2-20, 5-25, 5-15, or 5-10 bases in length. In some instances, the indels are not more than 1, 2, 3, 5, 7, 8, 10, 12, 15, 17, 20, 25, or no more than 50 bases in length. In some instances, a variant described herein is located in a gene. In some instances, a library described herein comprises variant sequences found in at least 2, 5, 10, 15, 20, 25, 30, 50, 60, 75, 100, 125, 150, 200, 250, 300, 400, or at least 500 genes. In some instances, a library described herein comprises variant sequences found in about 2, 5, 10, 15, 20, 25, 30, 50, 60, 75, 100, 125, 150, 200, 250, 300, 400, or about 500 genes. In some instances, a library described herein comprises variant sequences found in 5-500, 5-100, 5-50, 10-200, 10-100, 25-500, 25-250, 25-150, 50-150, 50-250, 50-500, or 75-500 genes.

In some embodiments, identification of variant sequences is accomplished using imputed data. In some instances, identification of variant sequences near a known or detected variant sequence inform the identity of a variant sequence not measured, or which lacks sequencing data to accurately call. In some instances, the unmeasured (or unknown) genomic variant is within 100 bases, 500 bases, 1,000 bases, 10,000 bases, 100,000 bases, or 1,000,000 bases of a measured (or identified) genomic variant or variants, or more, depending on linkage disequilibrium (the non-random association of alleles for different variants within a population) between measured and unmeasured variants. In some instances, linkage disequilibrium may be inferred by making use of information about recombination rates observed in a genome or population otherwise known genetic distance. In some instances, recombination rates, genetic distance maps, and variants themselves may vary between different populations.

Variants may be present in a population of individuals, a single individual, tissue, or other group at different frequencies, such as in a genome. In some instances, genomic variants are co-occurring in less than 0.001, 0.01, 0.1, 0.5, 1, 1.5, 2, 5, 10, 20, 25, 50, or 75% of individuals in a group. In some instances, genomic variants are co-occurring in more than 0.001, 0.01, 0.1, 0.5, 1, 1.5, 2, 5, 10, 20, 25, 50, or 75% of individuals in a group. In some instances, genomic variants are co-occurring in about 0.001, 0.01, 0.1, 0.5, 1, 1.5, 2, 5, 10, 20, 25, 50, or 75% of individuals in a group. In some instances, genomic variants are co-occurring in 0.1-10%, 0.001-10%, 0.01-10%, 0.01-1%, 0.001-1%, 0.1-25%, 0.1-10%, or 0.1-5% of individuals in a group. In some instances, the occurrence of a variant is called a variant allele frequency (VAF).

Described herein are variant sequences for detecting a disease or condition. In some instances, the disease or condition is a proliferative disease. In some instances, the disease or condition is associated with low variant allele frequencies, such as minimal residual disease (MRD). In some instances, the disease or condition is a viral or bacterial disease or condition. In some instances, the disease or condition is cancer. In some instances, the variant sequence is present in an oncogene or tumor suppressor gene. In some instances, the variant sequence is present in one or more of genes ABL1, ABL2, AKT1, ALK, APC, AR, ARAF, ARIDIA, ATM, ATR, BAP1, BRAF, BRCA1, BRCA2, CCND1, CDC6, CDH1, CDK12, CDK4, CDX2, CTNNB1, DDR2, EGFR, EML4, ERBB2, ERBB3, ERG, ESR1, EZH2, FBXW7, FGFR1, FGFR2, FGFR3, FLT3, FOXA1, FOXL2, GATA3, GNA11, GNAQ, GNAS, HNFIA, HRAS, IDH1, IDH2, JAK2, KDM5C, KDM6A, KIF5B, KIT, KRAS, MAP2K1, MAPK1, MET, MIR4728, ERBB2, MLH1, MPL, MYCN, MYD88, NCOA4, NF1, NF2, NFE2L2, NOTCH1, NPM1, NRAS, PBRM1, PDGFRA, PIK3CA, PTEN, PTPN11, RET, RHEB, RHOA, RIT1, ROS1, SETD2, SMAD4, SMO, SPOP, TERT, TMPRSS2, TP53, TPR, TSC1, and VHL. In some instances, the variant sequence is present in one, two, three, five, seven, ten, 15, 20, 25, or more of genes ABL1, ABL2, AKT1, ALK, APC, AR, ARAF, ARIDIA, ATM, ATR, BAP1, BRAF, BRCA1, BRCA2, CCND1, CDC6, CDH1, CDK12, CDK4, CDX2, CTNNB1, DDR2, EGFR, EML4, ERBB2, ERBB3, ERG, ESR1, EZH2, FBXW7, FGFR1, FGFR2, FGFR3, FLT3, FOXA1, FOXL2, GATA3, GNA11, GNAQ, GNAS, HNFIA, HRAS, IDH1, IDH2, JAK2, KDM5C, KDM6A, KIF5B, KIT, KRAS, MAP2K1, MAPK1, MET, MIR4728, ERBB2, MLH1, MPL, MYCN, MYD88, NCOA4, NF1, NF2, NFE2L2, NOTCH1, NPM1, NRAS, PBRM1, PDGFRA, PIK3CA, PTEN, PTPN11, RET, RHEB, RHOA, RIT1, ROS1, SETD2, SMAD4, SMO, SPOP, TERT, TMPRSS2, TP53, TPR, TSC1, and VHL. In some instances, multiple variant sequences are present in a single gene. In some instances, a variant sequence is present in one, two, three, five, seven, ten, 15, 20, 25 or more of genes. In some instances, a variant sequence is present in one, two, three, five, seven, ten, 15, 20, 25 or more of genes which are associated with a disease or condition.

In some instances, the disease or condition is breast cancer. In some instances, the variant sequence is present in one or more of genes TP53, PIK3CA, ERBB2, MYC, FGFR1/ZNF703, GATA3, CCND1, and CHD1 (e.g., CDH1*).

In some instances, the disease or condition is lung cancer. In some instances, the variant sequence is present in one or more of genes KRAS (e.g., K117N), EGFR, ROS, ALK, and BRAF.

In some instances, the disease or condition is colorectal cancer. In some instances, the variant sequence is present in one or more of genes TP53 APC, KRAS, BRAF, PIK3CA, SMAD4, FBXW7 (e.g., R465C), and NF1.

In some instances, the disease or condition is bladder cancer. In some instances, the variant sequence is present in one or more of TP53, FGFR3 (e.g., S249C), ARID1A and KDM6A.

In some instances, the disease or condition is prostate cancer. In some instances, the variant sequence is present in one or more of genes ETS (e.g., ETS-TMPRSS2), SPOP (e.g., F133V), TP53, FOXA1 (e.g., R219), and PTEN.

In some instances, the disease or condition is kidney cancer. In some instances, the variant sequence is present in one or more of genes PBRM1, SETD2, BAP1, KDM5C, MTOR, VHL, MET, NF2, KDM6A, SMARCB1, FH, and CDKN2A.

In some instances, the disease or condition is melanoma. In some instances, the variant sequence is present in one or more of genes NRAS, BRAF, PTEN, CDKN2A, MAP2K1, MAP2K2, GNAQ, GNA11, BAP (e.g., W196X).

In some instances, the variant sequence is a variant is described in Table 1, below.

TABLE 1

| | Mutation | | | | | | |
|---|---|---|---|---|---|---|---|
| Gene | Description | COSMIC_id | Chrom | Pos | Shift | Ref | Alt |
| ARID1A | Q1401* | COSM51417 | chr1 | 26774428 | | C | T |
| ARID1A | M1564Hfs*8 | COSM211769 | chr1 | 26774915 | 487 | C | CC |
| MPL | p.W515L | COSM18918 | chr1 | 43349338 | 16574423 | G | T |
| NRAS | Q61H | COSM586 | chr1 | 114713907 | 71364569 | T | G |
| NRAS | G12D | COSM564 | chr1 | 114716126 | 2219 | C | T |
| RIT1 | M90I | COSM357927 | chr1 | 155904470 | 41188344 | C | T |
| ABL2 | P986Hfs*4 | COSM2095020 | chr1 | 179108313 | 23203843 | GG | G |
| DDR2 | I638F | COSM7363943 | chr1 | 162775707 | −16332606 | A | T |
| GATA3 | P408fs | COSM166059 | chr10 | 8073911 | −154701796 | C | CG |
| RET | p.M918T | COSM965 | chr10 | 43121968 | 35048057 | T | C |
| PTEN | R130G | COSM5033 | chr10 | 87933148 | 44811180 | G | A |
| PTEN | p.D268Gfs*30 | COSM5012 | chr10 | 87958018 | 24870 | A | AA |
| PTEN | p.N323Kfs*2 | COSM4990 | chr10 | 87961060 | 3042 | A | AA |
| FGFR2 | K659E | COSM36909 | chr10 | 121488002 | 33526942 | T | C |
| FGFR2 | N549K | COSM36912 | chr10 | 121498520 | 10518 | A | T |
| FGFR2 | C382R | COSM36906 | chr10 | 121515260 | 16740 | A | G |
| FGFR2 | S252W | COSM36903 | chr10 | 121520163 | 4903 | G | C |
| HRAS | G12V | COSM483 | chr11 | 534288 | −120985875 | C | A |
| CCND1 | T286I | COSM931395 | chr11 | 69651251 | 69116963 | C | T |
| CCND1 | E275*fs | COSM931393 | chr11 | 69651217 | 69116929 | G | T |
| ATM | S214Pfs*16 | COSM1350740 | chr11 | 108244089 | 38592838 | CT | C |
| KRAS | K117N | COSM19940 | chr12 | 25225713 | −83018376 | T | G |
| KRAS | Q61H | COSM554 | chr12 | 25227341 | 1628 | T | G |
| KRAS | G13D | COSM532 | chr12 | 25245347 | 18006 | C | T |
| KRAS | p.G12D | COSM521 | chr12 | 25245350 | 3 | C | T |
| ERBB3 | v104m | COSM20710 | chr12 | 56085070 | 30839723 | G | A |
| CDK4 | R24C | COSM1677139 | chr12 | 57751648 | 1666578 | G | A |
| PTPN11 | E76K | COSM13000 | chr12 | 112450406 | 54698758 | G | A |
| PTPN11 | G503R | COSM14259 | chr12 | 112489083 | 38677 | G | A |
| HNF1A | P289fs | COSM1476243 | chr12 | 120994313 | 8505230 | G | GC |
| CDX2 | V306Cfs*2 | COSM1366182 | chr13 | 27963146 | −93031167 | CC | C |
| FLT3 | p.D835Y | COSM783 | chr13 | 28018505 | 55359 | C | A |
| BRCA2 | N1784Tfs*7 | COSM18607 | chr13 | 32339699 | 4321194 | CA | C |
| BRCA2 | T3033Lfs*29 | COSM1366491 | chr13 | 32379885 | 40186 | CA | C |
| FOXA1 | R219S | COSM3738526 | chr14 | 37592129 | 5212244 | G | T |
| AKT1 | Q79K | COSM159008 | chr14 | 104776711 | 67184582 | G | T |
| AKT1 | L52R | COSM93893 | chr14 | 104780108 | 3397 | A | C |
| AKT1 | p.E17K | COSM33765 | chr14 | 104780214 | 3503 | C | T |
| MAP2K1 | K57N | COSM1235478 | chr15 | 66435117 | −38345097 | G | T |
| IDH2 | R140Q | COSM41590 | chr15 | 90088702 | 23653585 | C | T |
| IDH2 | R172K | COSM33733 | chr15 | 90088606 | 23653489 | C | T |
| CDH1 | Q23* | COSM19503 | chr16 | 68738315 | −21350387 | C | T |
| CDH1 | A634V | COSM19822 | chr16 | 68822190 | 83875 | C | T |
| CDH1 | R732Q | COSM972800 | chr16 | 68828204 | 6014 | G | A |
| TP53 | R282W | COSM10704 | chr17 | 7673776 | −61154428 | G | A |
| TP53 | R342Efs*3 | COSM18597 | chr17 | 7670685 | −61157519 | GG | G |
| TP53 | p.R273H | COSM10660 | chr17 | 7673802 | 3117 | C | T |
| TP53 | G245C | COSM11081 | chr17 | 7674230 | 428 | C | A |
| TP53 | p.R175H | COSM10648 | chr17 | 7675088 | 858 | C | T |
| TP53 | p.L26Pfs*11 | COSM45386 | chr17 | 7676382 | 1294 | CAGAA | C |
| | | | | | | CGTTG | |
| | | | | | | TTTTC | |
| | | | | | | AGGAA | |
| | | | | | | GT | |
| | | | | | | (SEQ ID | |
| | | | | | | NO: 5) | |
| TP53 | R209Kfs*6 | COSM6482 | chr17 | 7674903 | 673 | TTC | T |
| TP53 | P152Rfs*18 | COSM43792 | chr17 | 7675156 | 68 | CG | C |
| TP53 | V73Rfs*76 | COSM128714 | chr17 | 7676152 | 996 | C | C |
| NF1 | I679Dfs*21 | COSM24504 | chr17 | 31226465 | 23550083 | C | CC |
| NF1 | F1247Ifs*18 | COSM436320 | chr17 | 31235638 | 9173 | CTGTT | C |
| NF1 | Y2285Tfs*5 | COSM39161 | chr17 | 31338733 | 103095 | CACTT | C |
| CDK12 | W719* | COSM118018 | chr17 | 39492798 | 8154065 | G | A |
| CDK12 | E928fs27* | COSM6965693 | chr17 | 39515745 | 22947 | A | AATACA |
| | | | | | | | CAAAGAT |
| | | | | | | | (SEQ ID |
| | | | | | | | NO: 6) |
| ERBB2 | L755S | COSM14060 | chr17 | 39723967 | 208222 | T | C |
| ERBB2 | p.A775_G776insYVMA | COSM20959 | chr17 | 39724730 | 763 | C | CATACG |
| | | | | | | | TGATGGC |
| | | | | | | | (SEQ ID |
| | | | | | | | NO: 7) |
| ERBB2 | P780_Y781insGSP | COSM21607 | chr17 | 39724758 | 28 | A | AGGCTC |
| | | | | | | | ACCA |
| | | | | | | | (SEQ ID |
| | | | | | | | NO: 8) |
| ERBB2 | V842I | COSM14065 | chr17 | 39725079 | 349 | G | A |
| BRCA1 | R1443* | COSM979730 | chr17 | 43082434 | 3357355 | G | A |
| BRCA1 | K654Sfs*47 | COSM219054 | chr17 | 43093569 | 11135 | CT | C |
| BRCA1 | E23Vfs*17 | COSM35893 | chr17 | 43124027 | 30458 | ACT | A |
| SPOP | F133V | COSM219965 | chr17 | 49619064 | 6495037 | A | C |
| SMAD4 | D52Rfs*2 | COSM14091 | chr18 | 51047198 | 1428134 | A | AA |
| SMAD4 | R361H | COSM14122 | chr18 | 51065549 | 18351 | G | A |
| NFE2L2 | G333C | COSM1193323 | chr19 | 10491905 | −40573644 | C | A |
| MYCN | P44L | COSM35624 | chr2 | 15942195 | 5450290 | C | T |
| ALK | P1543S | COSM2941442 | chr2 | 29193460 | 13251265 | G | A |
| ALK | R1275Q | COSM28056 | chr2 | 29209798 | 16338 | C | T |
| ALK | R1192P | COSM7340824 | chr2 | 29220776 | 10978 | C | G |
| ALK | F1174L | COSM28055 | chr2 | 29220829 | 11031 | G | T |
| ALK | G1128A | COSM98475 | chr2 | 29222584 | 1755 | C | G |
| IDH1 | p.R132C | COSM28747 | chr2 | 208248389 | 179025805 | G | A |
| GNAS | p.R201C | COSM27887 | chr20 | 58909365 | −149339024 | C | T |
| MAPK1 | E322K | COSM461148 | chr22 | 21772875 | −37136490 | C | T |
| NF2 | L14Qfs*34 | COSM22312 | chr22 | 29604033 | 7831158 | GCT | G |
| NF2 | P275Tfs*4 | COSM6951489 | chr22 | 29665000 | 60967 | A | AA |
| NF2 | R341* | COSM21990 | chr22 | 29671847 | 6847 | C | T |
| NF2 | E445Gfs*9 | COSM22271 | chr22 | 29673477 | 1630 | CAGAG | C |
| VHL | F148Lfs*11 | COSM14410 | chr3 | 10146612 | −19526865 | AT | A |
| VHL | R161* | COSM17612 | chr3 | 10149804 | 3192 | C | T |
| MLH1 | R498fs | COSM5895322 | chr3 | 37028864 | 26879060 | GG | G |
| MYD88 | L265P | COSM85940 | chr3 | 38141150 | 1112286 | T | C |
| CTNNB1 | p.T41A | COSM5664 | chr3 | 41224633 | 3083483 | A | G |
| CTNNB1 | G34E | COSM5671 | chr3 | 41224613 | 3083463 | G | A |
| SETD2 | S2382Lfs*29 | COSM3068849 | chr3 | 47042655 | 5818022 | AG | A |
| SETD2 | R1407Gfs*5 | COSM3069036 | chr3 | 47120416 | 77761 | CT | C |
| SETD2 | S203Ifs*33 | COSM1161887 | chr3 | 47124027 | 3611 | TGA | T |
| RHOA | Y42C | COSM2849892 | chr3 | 49375465 | 2251438 | T | C |
| BAP1 | W196* | COSM51977 | chr3 | 52406900 | 3031435 | C | T |
| PBRM1 | p.1279Yfs*4 | COSM52863 | chr3 | 52644767 | 237867 | AT | A |
| FOXL2 | p.C134W | COSM33661 | chr3 | 138946321 | 86301554 | G | C |
| ATR | I774Yfs*5 | COSM214499 | chr3 | 142555906 | 3609585 | TT | T |
| PIK3CA | G106_R108del | COSM13475 | chr3 | 179199140 | 36643234 | AGGCA | A |
| | | | | | | ACCGT | |
| | | | | | | (SEQ ID | |
| | | | | | | NO: 9) | |
| PIK3CA | N345K | COSM754 | chr3 | 179203765 | 4625 | T | A |
| PIK3CA | p.E545K | COSM763 | chr3 | 179218303 | 14538 | G | A |
| PIK3CA | p.S553Tfs*20 | COSM27488 | chr3 | 179218327 | 24 | AGT | A |
| PIK3CA | p.H1047R | COSM775 | chr3 | 179234297 | 15994 | A | G |
| FGFR3 | p.S249C | COSM715 | chr4 | 1801841 | −177432456 | C | G |
| FGFR3 | Y375C | COSM718 | chr4 | 1804372 | 2531 | A | G |
| FGFR3 | K652E | COSM719 | chr4 | 1806162 | 1790 | A | G |
| FGFR3 | S249C | COSM715 | chr4 | 1801841 | −177432456 | C | G |
| PDGFRA | S566_E571delinsR | COSM30546 | chr4 | 54274884 | 52468722 | GCCCA | G |
| | | | | | | GATGG | |
| | | | | | | ACATGA | |
| | | | | | | (SEQ ID | |
| | | | | | | NO: 10) | |
| PDGFRA | N659K | COSM22414 | chr4 | 54277981 | 3097 | C | G |
| PDGFRA | p.D842V | COSM736 | chr4 | 54285926 | 7945 | A | T |
| PDGFRA | V561D | COSM739 | chr4 | 54274869 | 52468707 | T | A |
| KIT | L576P | COSM1290 | chr4 | 54727495 | 441569 | T | C |
| KIT | del557-558 | COSM1210 | chr4 | 54727434 | 441508 | CAGTG | C |
| | | | | | | GA | |
| KIT | K642E | COSM1304 | chr4 | 54728055 | 560 | A | G |
| KIT | p.D816V | COSM1314 | chr4 | 54733155 | 5100 | A | I |
| FBXW7 | R465C | COSM22932 | chr4 | 152328233 | 97595078 | G | A |
| TERT | C228G | tert_c228g | chr5 | 1295229 | −151033004 | G | A |
| TERT | C250T | None | chr5 | 1295250 | 22 | C | T |
| APC | p.R213* | COSM13134 | chr5 | 112780895 | 111485666 | C | T |
| APC | A1002Gfs*6 | COSM5748894 | chr5 | 112838598 | 57703 | G | GG |
| APC | p.E1309Dfs*4 | COSM13113 | chr5 | 112839514 | 916 | TAAAAG | T |
| APC | p.R1450* | COSM13127 | chr5 | 112839942 | 428 | C | T |
| APC | R2714C | COSM2991126 | chr5 | 112843734 | 3792 | C | T |
| APC | p.S1465Wfs*3 | COSM13864 | chr5 | 112839978 | 36 | AAG | A |
| NPM1 | p.W288fs*12 | COSM17559 | chr5 | 171410539 | 58566805 | C | CTCTG |
| ROS1 | G2032R | COSM1651690 | chr6 | 117317184 | −54093355 | C | T |
| ESR1 | D538G | COSM94250 | chr6 | 152098791 | 34781607 | A | G |
| EGFR | L718Q | COSM6503269 | chr7 | 55174012 | −96924779 | T | A |
| EGFR | p.E746_A750 | COSM6225 | chr7 | 55174772 | 760 | GGAAT | G |
| | delELREA | | | | | TAAGA | |
| | | | | | | GAAGCA | |
| | | | | | | (SEQ ID | |
| | | | | | | NO: 11) | |
| EGFR | S768I | COSM6241 | chr7 | 55181312 | 6540 | G | T |
| EGFR | p.D770_N77linsG | COSM12378 | chr7 | 55181319 | 7 | C | CGGT |
| EGFR | p.T790M | COSM6240 | chr7 | 55181378 | 6606 | C | T |
| EGFR | p.L858R | COSM6224 | chr7 | 55191822 | 10444 | T | G |
| EGFR | G724S | COSM13979 | chr7 | 55174029 | 17 | G | A |
| EGFR | L792H | COSM6493934 | chr7 | 55181384 | 6 | T | A |
| MET | exon_14_skip | met_exon14_skip | chr7 | 116771990 | 61580168 | G | A |
| MET | d1246n | COSM5015794 | chr7 | 116783353 | 11363 | G | A |
| SMO | D473H | COSM34198 | chr7 | 129209348 | 12425995 | G | C |
| BRAF | p.V600E | COSM476 | chr7 | 140753336 | 11543988 | A | T |
| EZH2 | Y641F | COSM37028 | chr7 | 148811635 | 8058299 | I | A |
| RHEB | Y35N | COSM485065 | chr7 | 151490964 | 2679329 | A | T |
| FGFR1 | K656E | COSM35673 | chr8 | 38414790 | −113076174 | T | C |
| FGFR1 | N546K | COSM19176 | chr8 | 38417331 | 2541 | G | T |
| JAK2 | p.V617F | COSM12600 | chr9 | 5073770 | −33343561 | G | T |
| GNAQ | p.Q209P | COSM28758 | chr9 | 77794572 | 72720802 | T | G |
| GNAQ | T96S | COSM404628 | chr9 | 77922196 | 127624 | T | A |
| ABL1 | F317V | COSM211607 | chr9 | 130872901 | 52950705 | T | G |
| TSC1 | Q794 | COSM753312 | chr9 | 132902616 | 2029715 | G | A |
| NOTCH1 | P2514Rfs*4 | COSM12774 | chr9 | 136496196 | 3593580 | CAG | C |
| NOTCH1 | p.L1600Pfs*10 | COSM5751249 | chr9 | 136504893 | 8697 | G | GG |
| KDM6A | K1097Sfs*6 | COSM7211707 | chrX | 45083464 | −91421429 | AAGTT | A |
| ARAF | S214C | COSM5044705 | chrX | 47566722 | 2483258 | C | G |
| KDM5C | p.D1407Tfs*5 | COSM1161909 | chrX | 53193534 | 5626812 | TC | T |
| AR | W742C | COSM5944171 | chrX | 67717530 | 14523996 | G | C |
| AR | T878A | COSM236693 | chrX | 67723710 | 6180 | A | G |

In some instances, the variant sequence is a variant described in Table 2, below.

TABLE 2

| | | | | | | | LEGACY | | |
|---|---|---|---|---|---|---|---|---|---|
| | COSMIC_ | Region_ | | | | | MUTATION_ | | |
| Gene | id | str | Chrom | Pos | Ref | Alt | ID | HGVSP | Name |
| RET | COSM9358963 | chr10: | chr10 | 43112867 | T | TTCC | COSM9358963 | ENSP00000347942.3: | T > TTCC |
| | | 43112852- | | | | | | p.Phe555_ | |
| | | 43112963 | | | | | | Ser556insLeu | |
| PTEN | COSM7350864 | chr10: | chr10 | 87864536 | T | TCGGG | COSM7350864 | ENSP00000361021.3: | T > TCGGG |
| | | 87864469- | | | | AGC | | p.Leu23SerfsTer23 | AGC |
| | | 87864548 | | | | | | | |
| PTEN | COSM5346960 | chr 10: | chr10 | 87894056 | T | TATGG | COSM5346960 | ENSP00000361021.3: | T > TATGG |
| | | 87894024- | | | | GATTG | | p.Phe37_ | GATTG |
| | | 87894109 | | | | (SEQ | | Pro38 | |
| | | | | | | ID | | insMetGlyLeu | |
| | | | | | | NO: | | | |
| | | | | | | 12) | | | |
| PTEN | COSM5882 | chr10: | chr10 | 87925549 | A | ATAT | COSM5882 | ENSP00000361021.3: | A > ATAT |
| | | 87925512- | | | | | | p.Tyr68dup | |
| | | 87925557 | | | | | | | |
| PTEN | COSM1173605 | chr 10: | chr10 | 87931069 | C | CTA | COSM1173605 | ENSP00000361021.3: | C > CTA |
| | | 87931045- | | | | | | p.Ala79ThrfsTer21 | |
| | | 87931089 | | | | | | | |
| PTEN | COSM7347202 | chr10: | chr10 | 87952180 | T | TCACC | COSM7347202 | ENSP00000361021.3: | T > TCACC |
| | | 87952117- | | | | GA | | p.His185_ | GA |
| | | 87952259 | | | | | | Leu186 | |
| | | | | | | | | insHisArg | |
| ATM | COSM1235448 | chr11: | chr11 | 108247062 | T | TA | COSM1235448 | ENSP00000278616.4: | T > TA |
| | | 108246963- | | | | | | p.Ser334 | |
| | | 108247127 | | | | | | TyrfsTer5 | |
| ATM | COSM6928114 | chr11: | chr11 | 108256298 | TGAAG | TAAAA | COSM6928114 | ENSP00000278616.4: | TGAAG |
| | | 108256214- | | | | A | | p.Glu737LysfsTer6 | > TAAAA |
| | | 108256340 | | | | | | | A |
| ATM | COSM9312366 | chr11: | chr11 | 108292758 | C | CATAA | COSM9312366 | ENSP00000278616.4: | C > CATAA |
| | | 108292618- | | | | | | p.Pro1526HisfsTer6 | |
| | | 108292793 | | | | | | | |
| ATM | COSM9358682 | chr11: | chr11 | 108293339 | G | GGATA | COSM9358682 | ENSP00000278616.4: | G > GGATA |
| | | 108293312- | | | | | | p.Ile1547AspfsTer4 | |
| | | 108293477 | | | | | | | |
| ATM, | COSM6853938 | chr11: | chr11 | 108331534 | G | GA | COSM6853938 | ENSP00000278616.4: | G > GA |
| C11orf65 | | 108331443- | | | | | | p.Gly2536GlufsTer4 | |
| | | 108331557 | | | | | | | |
| ATM, | COSM6936524 | chr11: | chr11 | 108331960 | T | TA | COSM6936524 | ENSP00000278616.4: | T > TA |
| C11orf65 | | 108331878- | | | | | | p.Phe2571TyrfsTer4 | |
| | | 108332037 | | | | | | | |
| ATM, | COSM6854263 | chr11: | chr11 | 108345767 | G | GT | COSM6854263 | ENSP00000278616.4: | G > GT |
| C11orf65 | | 108345742- | | | | | | p.Glu2815ValfsTer4 | |
| | | 108345908 | | | | | | | |
| AKT1 | COSM7345039 | chr14: | chr14 | 104772908 | G | GC | COSM7345039 | ENSP00000451828.1: | G > GC |
| | | 104772877- | | | | | | p.Ser381CysfsTer54 | |
| | | 104773092 | | | | | | | |
| AKT1 | COSM5751911 | chr14: | chr14 | 104792618 | T | TC | COSM5751911 | ENSP00000451828.1: | T > TC |
| | | 104792597- | | | | | | p.Glu9GlyfsTer24 | |
| | | 104792643 | | | | | | | |
| ERBB2 | COSM9494270 | chr17: | chr17 | 39727873 | C | CAGAG | COSM9494270 | ENSP00000269571.4: | C > CAGAG |
| | | 39727688- | | | | | | p.Gln1200ArgfsTer78 | |
| | | 39728044 | | | | | | | |
| TP53 | COSM6503572 | chr17: | chr17 | 7669655 | C | CT | COSM6503572 | ENSP00000269305.4: | C > CT |
| | | 7669608- | | | | | | p.Arg379GInfsTer3 | |
| | | 7669690 | | | | | | | |
| ALK | COSM7347227 | chr2: | chr2 | 29320797 | T | TG | COSM7347227 | ENSP00000373700.3: | T > TG |
| | | 29320750- | | | | | | p.Gln500HisfsTer26 | |
| | | 29320882 | | | | | | | |
| PIK3CA | COSM5751700 | chr3: | chr3 | 179219582 | G | GT | COSM5751700 | ENSP00000263967.3: | G > GT |
| | | 179219570- | | | | | | p.Val587CysfsTer10 | |
| | | 179219735 | | | | | | | |
| CTNNB1 | COSM6853630 | chr3: | chr3 | 41233407 | G | GGGA | COSM6853630 | ENSP00000495360.1: | G > GGGA |
| | | 41233340- | | | | | | p.Trp383_ | |
| | | 41233444 | | | | | | Thr384insGlu | |
| FGFR3 | COSM13248 | chr4: | chr4 | 1804823 | G | GGTAA | COSM13248 | ENSP00000339824.4: | G > GGTAA |
| | | 1804823- | | | | CA | | p.Val425_ | CA |
| | | 1804969 | | | | | | Ser426insThrVal | |
| FGFR3 | COSM7448276 | chr4: | chr4 | 1806568 | T | TTGGG | COSM7448276 | ENSP00000339824.4: | T > TTGGG |
| | | 1806545- | | | | AGATC | | p.Trp687delins | AGATC |
| | | 1806683 | | | | TTGCA | | LeuGlyAspLeu | TTGCA |
| | | | | | | C | | AlaArg | C |
| | | | | | | (SEQ | | | |
| | | | | | | ID | | | |
| | | | | | | NO: | | | |
| | | | | | | 13) | | | |
| FGFR3 | COSM729 | chr4: | chr4 | 1807221 | CT | CGA | COSM729 | ENSP00000339824.4: | CT > CGA |
| | | 1807115- | | | | | | p.Leu796ArgfsTer23 | |
| | | 1807262 | | | | | | | |
| PDGFR | COSM7345286 | chr4: | chr4 | 54261354 | C | CA | COSM7345286 | ENSP00000257290.5: | C > CA |
| A | | 54261094- | | | | | | p.His104ThrfsTer8 | |
| | | 54261412 | | | | | | | |
| PDGFR | COSM9358924 | chr4: | chr4 | 54270721 | A | AAGCT | COSM9358924 | ENSP00000257290.5: | A > AAGCT |
| A | | 54270632- | | | | | | p.Ser404LysfsTer6 | |
| | | 54270748 | | | | | | | |
| KIT | COSM6008883 | chr4: | chr4 | 54723605 | A | ACGAT | COSM6008883 | ENSP00000288135.5: | A > ACGAT |
| | | 54723583- | | | | TTT | | p.Arg420PhefsTer30 | TTT |
| | | 54723698 | | | | | | | |
| KIT | COSM53306 | chr4: | chr4 | 54726012 | C | CTGCC | COSM53306 | ENSP00000288135.5: | C > CTGCC |
| | | 54725856- | | | | TT | | p.Ala502_ | TT |
| | | 54726050 | | | | | | Tyr503insPheAla | |
| APC | COSM9113053 | chr5: | chr5 | 112767380 | G | GGAGA | COSM9113053 | ENSP00000257430.4: | G > GGAGA |
| | | 112767188- | | | | AAGA | | p.Glu138GlyfsTer35 | AAGA |
| | | 112767390 | | | | | | | |
| APC | COSM5010340 | chr5: | chr5 | 112815593 | G | GATGT | COSM5010340 | ENSP00000257430.4: | G > GATGT |
| | | 112815494- | | | | TT | | p.Lys311_ | TT |
| | | 112815593 | | | | | | Val312insMetPhe | |
| APC | COSM25155 | chr5: | chr5 | 112819174 | C | CG | COSM25155 | ENSP00000257430.4: | C > CG |
| | | 112818965- | | | | | | p.Arg382GlnfsTer15 | |
| | | 112819344 | | | | | | | |
| APC | COSM6854200 | chr5: | chr5 | 112835141 | T | TA | COSM6854200 | ENSP00000257430.4: | T > TA |
| | | 112834950- | | | | | | p.Ile646AspfsTer5 | |
| | | 112835165 | | | | | | | |
| ROS1 | COSM6967149 | chr6: | chr6 | 117319975 | G | GA | COSM6967149 | ENSP00000357494.3: | G > GA |
| | | 117319867- | | | | | | p.Leu1945SerfsTer17 | |
| | | 117320030 | | | | | | | |
| ROS1 | COSM9499684 | chr6: | chr6 | 117359964 | C | CAA | COSM9499684 | ENSP00000357494.3: | C > CAA |
| | | 117359808- | | | | | | p.Val1165LeufsTer4 | |
| | | 117360011 | | | | | | | |
| MET | COSM6957131 | chr7: | chr7 | 116763127 | C | CA | COSM6957131 | ENSP00000317272.6: | C > CA |
| | | 116763049- | | | | | | p.Leu833ThrfsTer18 | |
| | | 116763268 | | | | | | | |
| RET | COSM7341796 | chr10: | chr10 | 43077301 | TTGC | T | COSM7341796 | ENSP00000347942.3: | TTGC > T |
| | | 43077258- | | | | | | p.Leu19del | |
| | | 43077331 | | | | | | | |
| RET | COSM7351211 | chr 10: | chr10 | 43102447 | CCTT | C | COSM7351211 | ENSP00000347942.3: | CCTT > C |
| | | 43102341- | | | | | | p.Phe150del | |
| | | 43102629 | | | | | | | |
| RET | COSM4989957 | chr 10: | chr10 | 43105037 | CGAGC | C | COSM4989957 | ENSP00000347942.3: | CGAGC |
| | | 43104951- | | | TGGT | | | p.Glu238GlyfsTer113 | TGGT > C |
| | | 43105193 | | | | | | | |
| RET | COSM9277092 | chr 10: | chr10 | 43111413 | GCAGA | G | COSM9277092 | ENSP00000347942.3: | GCAGA |
| | | 43111206- | | | CCTCT | | | p.Thr492_ | CCTCT |
| | | 43111465 | | | AGGCA | | | Gln499del | AGGCA |
| | | | | | GGCCC | | | | GGCCC |
| | | | | | AGGCC | | | | AGGCC |
| | | | | | (SEQ | | | | > G |
| | | | | | ID | | | | |
| | | | | | NO: | | | | |
| | | | | | 14) | | | | |
| RET | COSM1237681 | chr10: | chr10 | 43112100 | TGTGG | T | COSM1237681 | ENSP00000347942.3: | TGTGG |
| | | 43112098- | | | CCGAG | | | p.Val509_ | CCGAG |
| | | 43112224 | | | (SEQ | | | Glu511del | > T |
| | | | | | ID | | | | |
| | | | | | NO: | | | | |
| | | | | | 15) | | | | |
| RET | COSM984 | chr10: | chr10 | 43113625 | ACTGC | A | COSM984 | ENSP00000347942.3: | ACTGC |
| | | 43113555- | | | TTCCC | | | p.Phe612_ | TTCCC |
| | | 43113675 | | | TGAGG | | | Cys620del | TGAGG |
| | | | | | AGGAG | | | | AGGAG |
| | | | | | AAGTG | | | | AAGTG |
| | | | | | CTT | | | | CTT > A |
| | | | | | (SEQ | | | | |
| | | | | | ID | | | | |
| | | | | | NO: | | | | |
| | | | | | 16) | | | | |
| RET | COSM962 | chr10: | chr10 | 43120163 | GAGAT | G | COSM962 | ENSP00000347942.3: | GAGAT |
| | | 43120080- | | | GTTTA | | | p.Asp898_ | GTTTA |
| | | 43120203 | | | TGA | | | Glu901del | TGA > G |
| | | | | | (SEQ | | | | |
| | | | | | ID | | | | |
| | | | | | NO: | | | | |
| | | | | | 17) | | | | |
| RET | COSM6929334 | chr 10: | chr10 | 43123710 | AG | A | COSM6929334 | ENSP00000347942.3: | AG > A |
| | | 43123670- | | | | | | p.Gly949GlufsTer16 | |
| | | 43123808 | | | | | | | |
| RET | COSM7449721 | chr10: | chr10 | 43128223 | TGCTT | T | COSM7449721 | ENSP00000347942.3: | TGCTT |
| | | 43128111- | | | TCACC | | | p.Leu1101GlnfsTer3 | TCACC |
| | | 43128269 | | | CTCAG | | | | CT |
| | | | | | CG | | | | CAGCG |
| | | | | | (SEQ | | | | > T |
| | | | | | ID | | | | |
| | | | | | NO: | | | | |
| | | | | | 18) | | | | |
| PTEN | COSM6942496 | chr10: | chr10 | 87965289 | GAAGC | G | COSM6942496 | ENSP00000361021.3: | GAAGC |
| | | 87965286- | | | TGTAC | | | p.Leu345GlnfsTer2 | TGTAC |
| | | 87965472 | | | TTCAC | | | | TTCAC |
| | | | | | AA | | | | AA > G |
| | | | | | (SEQ | | | | |
| | | | | | ID | | | | |
| | | | | | NO: | | | | |
| | | | | | 19) | | | | |
| ATM | COSM1315819 | chr11: | chr11 | 108227624 | CATGA | C | COSM1315819 | ENSP00000278616.4: | CATGA |
| | | 108227624- | | | GTCTA | | | p.Met1? | GTCTA |
| | | 108227696 | | | GTACT | | | | GTACT |
| | | | | | TAATG | | | | TAATG |
| | | | | | (SEQ | | | | > C |
| | | | | | ID | | | | |
| | | | | | NO: | | | | |
| | | | | | 20) | | | | |
| ATM | COSM6978979 | chr11: | chr11 | 108229315 | CAAAC | C | COSM6978979 | ENSP00000278616.4: | CAAAC |
| | | 108229177- | | | AGAA | | | p.Asn109SerfsTer3 | AGAA > C |
| | | 108229323 | | | | | | | |
| ATM | COSM758337 | chr11: | chr11 | 108235814 | TATCT | T | COSM758337 | ENSP00000278616.4: | TATCT |
| | | 108235669- | | | C | | | p.Ser160AlafsTer23 | C > T |
| | | 108235834 | | | | | | | |
| ATM | COSM3733253 | chr11: | chr11 | 108244952 | AAG | A | COSM3733253 | ENSP00000278616.4: | AAG > A |
| | | 108244787- | | | | | | p.Glu277SerfsTer4 | |
| | | 108245026 | | | | | | | |
| ATM | COSM1235427 | chr11: | chr11 | 108249045 | GGGAA | G | COSM1235427 | ENSP00000278616.4: | GGGAA |
| | | 108248932- | | | GTA | | | p.Trp393Ter | GTA > G |
| | | 108249102 | | | | | | | |
| ATM | COSM6945044 | chr11: | chr11 | 108250727 | CAAAG | CT | COSM6945044 | ENSP00000278616.4: | CAAAG |
| | | 108250700- | | | | | | p.Lys422del | > CT |
| | | 108251072 | | | | | | | |
| ATM | COSM6935895 | chr11: | chr11 | 108251945 | AAAGG | A | COSM6935895 | ENSP00000278616.4: | AAAGG |
| | | 108251836- | | | AATC | | | p.Lys573AsnfsTer13 | AATC > A |
| | | 108252031 | | | | | | | |
| ATM | COSM6958308 | chr11: | chr11 | 108252838 | GGA | G | COSM6958308 | ENSP00000278616.4: | GGA > G |
| | | 108252816- | | | | | | p.Lys610AsnfsTer11 | |
| | | 108252912 | | | | | | | |
| ATM | COSM22531 | chr11: | chr11 | 108253991 | CTGTC | CG | COSM22531 | ENSP00000278616.4: | CTGTC |
| | | 108253813- | | | TTCTG | | | p.Cys693_ | TTCTG |
| | | 108254039 | | | GGATT | | | Gln700delinsGlu | GGATT |
| | | | | | ATCAG | | | | ATCAG |
| | | | | | AAC | | | | AAC > CG |
| | | | | | (SEQ | | | | |
| | | | | | ID | | | | |
| | | | | | NO: | | | | |
| | | | | | 21) | | | | |
| ATM | COSM6986880 | chr11: | chr11 | 108257598 | TGTAC | T | COSM6986880 | ENSP00000278616.4: | TGTAC |
| | | 108257480- | | | CA | | | p.Cys790_ | CA > T |
| | | 108257606 | | | | | | Lys792delinsTer | |
| ATM | COSM9179264 | chr11: | chr11 | 108259065 | GTAAA | G | COSM9179264 | | GTAAA |
| | | 108258985- | | | AGTTT | | | | AGTTT |
| | | 108259075 | | | AGTAA | | | | AGTAA |
| | | | | | GTA | | | | GTA > G |
| | | | | | (SEQ | | | | |
| | | | | | ID | | | | |
| | | | | | NO: | | | | |
| | | | | | 22) | | | | |
| ATM | COSM6856770 | chr11: | chr11 | 108267334 | GTACC | G | COSM6856770 | ENSP00000278616.4: | GTACC |
| | | 108267170- | | | A | | | p.Thr878ArgfsTer4 | A > G |
| | | 108267342 | | | | | | | |
| ATM | COSM6906886 | chr11: | chr11 | 108268563 | TTGAT | T | COSM6906886 | ENSP00000278616.4: | TTGAT |
| | | 108268409- | | | TCTAG | | | p.Asp932ArgfsTer32 | TCTAG |
| | | 108268609 | | | CACGC | | | | CACGC |
| | | | | | (SEQ | | | | > T |
| | | | | | ID | | | | |
| | | | | | NO: | | | | |
| | | | | | 23) | | | | |
| ATM | COSM7345428 | chr11: | chr11 | 108271283 | ATGTT | A | COSM7345428 | ENSP00000278616.4: | ATGTT |
| | | 108271250- | | | | | | p.Cys987LysfsTer4 | > A |
| | | 108271406 | | | | | | | |
| ATM | COSM7345432 | chr11: | chr11 | 108272761 | GGA | G | COSM7345432 | ENSP00000278616.4: | GGA > G |
| | | 108272721- | | | | | | p.Gly1065GlufsTer7 | |
| | | 108272852 | | | | | | | |
| ATM | COSM1235411 | chr11: | chr11 | 108279519 | TC | T | COSM1235411 | ENSP00000278616.4: | TC > T |
| | | 108279490- | | | | | | p.Arg1106GlyfsTer3 | |
| | | 108279608 | | | | | | | |
| ATM | COSM9493731 | chr11: | chr11 | 108281162 | GA | G | COSM9493731 | ENSP00000278616.4: | GA > G |
| | | 108280994- | | | | | | p.Lys1192ArgfsTer3 | |
| | | 108281168 | | | | | | | |
| ATM | COSM758341 | chr11: | chr11 | 108282852 | ACTAC | A | COSM758341 | ENSP00000278616.4: | ACTAC |
| | | 108282709- | | | ACAAA | | | p.Asn1240LysfsTer4 | ACAAA |
| | | 108282879 | | | TATTG | | | | TATTG |
| | | | | | AGG | | | | AGG > A |
| | | | | | (SEQ | | | | |
| | | | | | ID | | | | |
| | | | | | NO: | | | | |
| | | | | | 24) | | | | |
| ATM | COSM21638 | chr11: | chr11 | 108284389 | CAGAG | C | COSM21638 | ENSP00000278616.4: | CAGAG |
| | | 108284226- | | | ACA | | | p.Arg1304ValfsTer43 | ACA > C |
| | | 108284473 | | | | | | | |
| ATM | COSM6958310 | chr11: | chr11 | 108287644 | GTTA | G | COSM6958310 | ENSP00000278616.4: | GTTA > G |
| | | 108287599- | | | | | | p.Leu1348del | |
| | | 108287715 | | | | | | | |
| ATM | COSM6971320 | chr11: | chr11 | 108289010 | TC | T | COSM6971320 | ENSP00000278616.4: | TC > T |
| | | 108288976- | | | | | | p.Pro1382HisfsTer4 | |
| | | 108289103 | | | | | | | |
| ATM | COSM6956709 | chr11: | chr11 | 108289695 | CTGTT | C | COSM6956709 | ENSP00000278616.4: | CTGTT |
| | | 108289601- | | | | | | p.Phe1445LeufsTer5 | > C |
| | | 108289801 | | | | | | | |
| ATM | COSM22532 | chr11: | chr11 | 108294983 | TGAAG | TT | COSM22532 | ENSP00000278616.4: | TGAAG |
| | | 108294926- | | | GACTA | | | p.Glu1612_ | GACTA |
| | | 108295059 | | | AAGGA | | | Gln1620delinsTer | AAGGA |
| | | | | | TCTTC | | | | TCTTC |
| | | | | | GAAGA | | | | GAAGA |
| | | | | | C | | | | C > TT |
| | | | | | (SEQ | | | | |
| | | | | | ID | | | | |
| | | | | | NO: | | | | |
| | | | | | 25) | | | | |
| ATM | COSM4745906 | chr11: | chr11 | 108297369 | AAAAG | A | COSM4745906 | ENSP00000278616.4: | AAAAG |
| | | 108297286- | | | | | | p.Glu1666PhefsTer2 | > A |
| | | 108297382 | | | | | | | |
| ATM | COSM22533 | chr11: | chr11 | 108299754 | TTTCT | T | COSM22533 | ENSP00000278616.4: | TTTCT |
| | | 108299713- | | | C | | | p.Phe1683TyrfsTer7 | C > T |
| | | 108299885 | | | | | | | |
| ATM | COSM22526 | chr11: | chr11 | 108301670 | GTTAC | G | COSM22526 | ENSP00000278616.4: | GTTAC |
| | | 108301647- | | | CTGT | | | p.Thr1735GlufsTer11 | CTGT > G |
| | | 108301789 | | | | | | | |
| ATM | COSM7347299 | chr11: | chr11 | 108302857 | TAGA | T | COSM7347299 | ENSP00000278616.4: | TAGA > T |
| | | 108302852- | | | | | | p.Glu1776del | |
| | | 108303029 | | | | | | | |
| ATM | COSM9358193 | chr11: | chr11 | 108304685 | AC | A | COSM9358193 | ENSP00000278616.4: | AC > A |
| | | 108304674- | | | | | | p.Cys1838ValfsTer8 | |
| | | 108304852 | | | | | | | |
| ATM | COSM1315822 | chr11: | chr11 | 108307969 | TGAG | T | COSM1315822 | ENSP00000278616.4: | TGAG > T |
| | | 108307896- | | | | | | p.Met1916_ | |
| | | 108307984 | | | | | | Arg1917delinsIle | |
| ATM, | COSM5967541 | chr11: | chr11 | 108310286 | TAAGA | T | COSM5967541 | ENSP00000278616.4: | TAAGA |
| C11orf65 | | 108310159- | | | AAAGT | | | p.Lys1964ArgfsTer19 | AAAGT |
| | | 108310315 | | | ATGGA | | | | ATGGA |
| | | | | | TGATC | | | | TGATC |
| | | | | | AAG | | | | AAG > T |
| | | | | | (SEQ | | | | |
| | | | | | ID | | | | |
| | | | | | NO: | | | | |
| | | | | | 26) | | | | |
| ATM, | COSM1235422 | chr11: | chr11 | 108316060 | TA | T | COSM1235422 | ENSP00000278616.4: | TA > T |
| C11orf65 | | 108316010- | | | | | | p.Tyr2049LeufsTer33 | |
| | | 108316113 | | | | | | | |
| ATM, | COSM6944878 | chr11: | chr11 | 108317460 | GAAGA | GTC | COSM6944878 | ENSP00000278616.4: | GAAGA |
| C11orf65 | | 108317372- | | | ACT | | | p.Glu2096ValfsTer29 | ACT > GTC |
| | | 108317521 | | | | | | | |
| ATM, | COSM6911065 | chr11: | chr11 | 108319958 | GA | G | COSM6911065 | ENSP00000278616.4: | GA > G |
| C11orf65 | | 108319953- | | | | | | p.Val2119Ter | |
| | | 108320058 | | | | | | | |
| ATM, | COSM21644 | chr11: | chr11 | 108325443 | GAA | G | COSM21644 | ENSP00000278616.4: | GAA > G |
| C11orf65 | | 108325309- | | | | | | p.Lys2237GlyfsTer11 | |
| | | 108325544 | | | | | | | |
| ATM, | COSM6933908 | chr11: | chr11 | 108326152 | CA | C | COSM6933908 | ENSP00000278616.4: | CA > C |
| C11orf65 | | 108326057- | | | | | | p.Lys2303ArgfsTer7 | |
| | | 108326225 | | | | | | | |
| ATM, | COSM758343 | chr11: | chr11 | 108327657 | CTAAA | C | COSM758343 | ENSP00000278616.4: | CTAAA |
| C11orf65 | | 108327644- | | | ACT | | | p.Lys2331HisfsTer6 | ACT > C |
| | | 108327758 | | | | | | | |
| ATM, | COSM6977654 | chr11: | chr11 | 108329028 | AAG | A | COSM6977654 | ENSP00000278616.4: | AAG > A |
| C11orf65 | | 108329020- | | | | | | p.Glu2366AspfsTer6 | |
| | | 108329238 | | | | | | | |
| ATM, | COSM6986181 | chr11: | chr11 | 108330215 | TACAC | T | COSM6986181 | ENSP00000278616.4: | TACAC |
| C11orf65 | | 108330213- | | | | | | p.Tyr2437Ter | > T |
| | | 108330421 | | | | | | | |
| ATM, | COSM4745907 | chr11: | chr11 | 108332850 | CTTAT | C | COSM4745907 | ENSP00000278616.4: | CTTAT |
| C11orf65 | | 108332761- | | | A | | | p.Ile | A > C |
| | | 108332900 | | | | | | 2629SerfsTer25 | |
| ATM, | COSM6853895 | chr11: | chr11 | 108333892 | TA | T | COSM6853895 | ENSP00000278616.4: | TA > T |
| C11orf65 | | 108333885- | | | | | | p.Asn2646IlefsTer14 | |
| | | 108333968 | | | | | | | |
| ATM, | COSM6986871 | chr11: | chr11 | 108334992 | AAATC | A | COSM6986871 | ENSP00000278616.4: | AAATC |
| C11orf65 | | 108334968- | | | TGGTG | | | p.Asn2679SerfsTer9 | TGGTG |
| | | 108335109 | | | ACTAT | | | | ACTAT |
| | | | | | AC | | | | AC > A |
| | | | | | (SEQ | | | | |
| | | | | | ID | | | | |
| | | | | | NO: | | | | |
| | | | | | 27) | | | | |
| ATM, | COSM1235408 | chr11: | chr11 | 108343270 | ACTGT | AA | COSM1235408 | ENSP00000278616.4: | ACTGT |
| C11orf65 | | 108343221- | | | CCCCA | | | p.Thr2773AsnfsTer4 | CCCCA |
| | | 108343371 | | | TTGGT | | | | TTGGT |
| | | | | | GAAT | | | | GAAT > AA |
| | | | | | (SEQ | | | | |
| | | | | | ID | | | | |
| | | | | | NO: | | | | |
| | | | | | 28) | | | | |
| ATM, | COSM22484 | chr11: | chr11 | 108347306 | GACA | G | COSM22484 | ENSP00000278616.4: | GACA > G |
| C11orf65 | | 108347278- | | | | | | p.Arg2871_ | |
| | | 108347365 | | | | | | His2872delinsSer | |
| ATM, | COSM6933059 | chr11: | chr11 | 108353803 | TGAGA | T | COSM6933059 | ENSP00000278616.4: | TGAGA |
| | | 108353765- | | | CAGTT | | | p.Glu2904AspfsTer29 | CAGTT |
| C11orf65 | | 108353880 | | | CCTTT | | | | CCTTT |
| | | | | | TA | | | | TA > T |
| | | | | | (SEQ | | | | |
| | | | | | ID | | | | |
| | | | | | NO: | | | | |
| | | | | | 29) | | | | |
| ATM, | COSM6930780 | chr11: | chr11 | 108354854 | ACT | A | COSM6930780 | ENSP00000278616.4: | ACT > A |
| C11orf65 | | 108354810- | | | | | | p.Leu2945ValfsTer10 | |
| | | 108354874 | | | | | | | |
| ATM, | COSM3733420 | chr11: | chr11 | 108365362 | GTCT | G | COSM3733420 | ENSP00000278616.4: | GTCT > G |
| C11orf65 | | 108365324- | | | | | | p.Leu3010del | |
| | | 108365508 | | | | | | | |
| AKT1 | COSM9358172 | chr14: | chr14 | 104773279 | CA | C | COSM9358172 | ENSP00000451828.1: | CA > C |
| | | 104773250- | | | | | | p.Cys310AlafsTer33 | |
| | | 104773379 | | | | | | | |
| ERBB2 | COSM5967125 | chr17: | chr17 | 39707050 | TGCTC | T | COSM5967125 | ENSP00000269571.4: | TGCTC |
| | | 39706989- | | | CGCCA | | | p.Leu46AlafsTer40 | CGCCA |
| | | 39707141 | | | CCTCT | | | | CCTCT |
| | | | | | ACCAG | | | | ACCAG |
| | | | | | (SEQ | | | | > T |
| | | | | | ID | | | | |
| | | | | | NO: | | | | |
| | | | | | 30) | | | | |
| ERBB2 | COSM7345562 | chr17: | chr17 | 39708492 | TCC | T | COSM7345562 | ENSP00000269571.4: | TCC > T |
| | | 39708320- | | | | | | p.Pro134ArgfsTer66 | |
| | | 39708534 | | | | | | | |
| ERBB2 | COSM7345564 | chr17: | chr17 | 39710383 | GC | G | COSM7345564 | ENSP00000269571.4: | GC > G |
| | | 39710339- | | | | | | p.Pro269GInfsTer28 | |
| | | 39710481 | | | | | | | |
| ERBB2 | COSM6961097 | chr17: | chr17 | 39712401 | CAAG | C | COSM6961097 | ENSP00000269571.4: | CAAG > c |
| | | 39712321- | | | | | | p.Lys369del | |
| | | 39712448 | | | | | | | |
| ERBB2 | COSM9494227 | chr17: | chr17 | 39715878 | GCT | G | COSM9494227 | ENSP00000269571.4: | GCT > G |
| | | 39715739- | | | | | | p.Phe486SerfsTer80 | |
| | | 39715939 | | | | | | | |
| ERBB2 | COSM6974323 | chr17: | chr17 | 39717434 | GATGA | G | COSM6974323 | ENSP00000269571.4: | GATGA |
| | | 39717319- | | | GGAGG | | | p.Glu619GlnfsTer11 | GGAGG |
| | | 39717484 | | | GCGCA | | | | GCGCA |
| | | | | | TGCCA | | | | TGCCA |
| | | | | | GCCTT | | | | GCCTT |
| | | | | | GCCCC | | | | GCCCC |
| | | | | | (SEQ | | | | > G |
| | | | | | ID | | | | |
| | | | | | NO: | | | | |
| | | | | | 31) | | | | |
| ERBB2 | COSM7345566 | chr17: | chr17 | 39719798 | TG | T | COSM7345566 | ENSP00000269571.4: | TG > T |
| | | 39719786- | | | | | | p.Asp638MetfsTer14 | |
| | | 39719834 | | | | | | | |
| ERBB2 | COSM6973838 | chr17: | chr17 | 39723542 | TGGA | T | COSM6973838 | ENSP00000269571.4: | TGGA > T |
| | | 39723537- | | | | | | p.Glu698del | |
| | | 39723660 | | | | | | | |
| ERBB2 | COSM7345570 | chr17: | chr17 | 39725766 | CG | C | COSM7345570 | ENSP00000269571.4: | CG > C |
| | | 39725706- | | | | | | p.Glu930ArgfsTer24 | |
| | | 39725853 | | | | | | | |
| MIR4728, | COSM6865894 | chr17: | chr17 | 39726611 | TG | T | COSM6865894 | ENSP00000269571.4: | TG > T |
| ERBB2 | | 39726561- | | | | | | p.Glu975AsnfsTer85 | |
| | | 39726659 | | | | | | | |
| ERBB2 | COSM6865896 | chr17: | chr17 | 39727352 | TCTCC | T | COSM6865896 | ENSP00000269571.4: | TCTCC |
| | | 39727294- | | | ACTGG | | | p.Leu1075MetfsTer48 | ACTGG |
| | | 39727547 | | | CACCC | | | | CACCC |
| | | | | | TCCGA | | | | TCCGA |
| | | | | | AGGGG | | | | AGGGG |
| | | | | | CTGG | | | | CTGG > T |
| | | | | | (SEQ | | | | |
| | | | | | ID | | | | |
| | | | | | NO: | | | | |
| | | | | | 32) | | | | |
| GNA11 | COSM9232870 | chr19: | chr19 | 3114989 | CA | C | COSM9232870 | ENSP00000078429.3: | CA > C |
| | | 3114943- | | | | | | p.Thr175ProfsTer49 | |
| | | 3115072 | | | | | | | |
| GNA11 | COSM1392334 | chr19: | chr19 | 3118935 | TG | T | COSM1392334 | ENSP00000078429.3: | TG > T |
| | | 3118923- | | | | | | p.Gly208AlafsTer16 | |
| | | 3119053 | | | | | | | |
| GNA11 | COSM6342228 | chr19: | chr19 | 3121130 | TG | T | COSM6342228 | ENSP00000078429.3: | TG > T |
| | | 3120988- | | | | | | p.Lys345 ArgfsTer108 | |
| | | 3121179 | | | | | | | |
| GNAS | COSM9277149 | chr20: | chr20 | 58854492 | AGATC | A | COSM9277149 | ENSP00000360141.3: | AGATC |
| | | 58853265- | | | CCGAC | | | p.Thr415_ | CCGAC |
| | | 58855333 | | | TCCGG | | | Gly423del | TCCGG |
| | | | | | GACAG | | | | GACAG |
| | | | | | CACCA | | | | CACCA |
| | | | | | GCC | | | | GCC > A |
| | | | | | (SEQ | | | | |
| | | | | | ID | | | | |
| | | | | | NO: | | | | |
| | | | | | 33) | | | | |
| GNAS | COSM6984215 | chr20: | chr20 | 58905438 | ACGA | AG | COSM6984215 | ENSP00000360141.3: | ACGA > AG |
| | | 58905382- | | | | | | p.Tyr806Ter | |
| | | 58905480 | | | | | | | |
| ALK | COSM7347365 | chr2: | chr2 | 29228926 | CACCC | C | COSM7347365 | ENSP00000373700.3: | CACCC |
| | | 29228883- | | | CCTCC | | | p.Phe921_ | CCTCC |
| | | 29229066 | | | GAA | | | Gly924del | GAA > C |
| | | | | | (SEQ | | | | |
| | | | | | ID | | | | |
| | | | | | NO: | | | | |
| | | | | | 34) | | | | |
| ALK | COSM2941501 | chr2: | chr2 | 29233586 | AC | A | COSM2941501 | ENSP00000373700.3: | AC > A |
| | | 29233564- | | | | | | p.GIy822ValfsTer9 | |
| | | 29233696 | | | | | | | |
| ALK | COSM6926372 | chr2: | chr2 | 29275466 | TC | T | COSM6926372 | ENSP00000373700.3: | TC > T |
| | | 29275401- | | | | | | p.Gly616AspfsTer49 | |
| | | 29275496 | | | | | | | |
| ALK | COSM6922292 | chr2: | chr2 | 29318345 | TG | T | COSM6922292 | ENSP00000373700.3: | TG > T |
| | | 29318303- | | | | | | p.Ser536ValfsTer25 | |
| | | 29318404 | | | | | | | |
| ALK | COSM9358093 | chr2: | chr2 | 29920118 | CCTTG | CG | COSM9358093 | ENSP00000373700.3: | CCTTG |
| | | 29919992- | | | GCGAA | | | p.Trp176_ | GCGAA |
| | | 29920659 | | | TCCAC | | | Gly181delinsArg | TCCAC |
| | | | | | CA | | | | CA > CG |
| | | | | | (SEQ | | | | |
| | | | | | ID | | | | |
| | | | | | NO: | | | | |
| | | | | | 35) | | | | |
| PIK3CA | COSM5613085 | chr3: | chr3 | 179209674 | GT | G | COSM5613085 | ENSP00000263967.3: | GT > G |
| | | 179209594- | | | | | | p.Gly411AlafsTer17 | |
| | | 179209700 | | | | | | | |
| PIK3CA | COSM6940128 | chr3: | chr3 | 179210290 | AGAAG | A | COSM6940128 | ENSP00000263967.3: | AGAAG |
| | | 179210185- | | | ATTTG | | | p.Glu453_ | ATTTG |
| | | 179210338 | | | CTGAA | | | Thr462del | CTGAA |
| | | | | | CCCTA | | | | CCCTA |
| | | | | | TTGGT | | | | TTGGT |
| | | | | | GTTAC | | | | GTTAC |
| | | | | | T | | | | T > A |
| | | | | | (SEQ | | | | |
| | | | | | ID | | | | |
| | | | | | NO: | | | | |
| | | | | | 36) | | | | |
| PIK3CA | COSM6911769 | chr3: | chr3 | 179221134 | GAGA | G | COSM6911769 | ENSP00000263967.3: | GAGA > G |
| | | 179220985- | | | | | | p.Lys724del | |
| | | 179221157 | | | | | | | |
| CTNNB | COSM6845286 | chr3: | chr3 | 41225063 | TCATC | T | COSM6845286 | ENSP00000495360.1: | TCATC |
| 1 | | 41224953- | | | CCA | | | p.His118LeufsTer13 | CCA > T |
| | | 41225207 | | | | | | | |
| CTNNB | COSM6963932 | chr3: | chr3 | 41225731 | CTAAA | CA | COSM6963932 | ENSP00000495360.1: | CTAAA |
| 1 | | 41225659- | | | ATGGC | | | p.Lys270_ | ATGGC |
| | | 41225861 | | | AGTG | | | Val273del | AGTG > CA |
| | | | | | (SEQ | | | | |
| | | | | | ID | | | | |
| | | | | | NO: | | | | |
| | | | | | 37) | | | | |
| CTNNB | COSM5608170 | chr3: | chr3 | 41227274 | AAACT | A | COSM5608170 | ENSP00000495360.1: | AAACT |
| 1 | | 41227207- | | | | | | p.Lys335AsnfsTer9 | > A |
| | | 41227352 | | | | | | | |
| CTNNB | COSM6939570 | chr3: | chr3 | 41235755 | GTTGT | G | COSM6939570 | ENSP00000495360.1: | GTTGT |
| 1 | | 41235723- | | | ACC | | | p.Cys573GlufsTer6 | ACC > G |
| | | 41235843 | | | | | | | |
| CTNNB | COSM6853546 | chr3: | chr3 | 41236462 | TCTGA | T | COSM6853546 | ENSP00000495360.1: | TCTGA |
| 1 | | 41236348- | | | CAGAG | | | p.Thr641_ | CAGAG |
| | | 41236499 | | | TTA | | | Leu644del | TTA > T |
| | | | | | (SEQ | | | | |
| | | | | | ID | | | | |
| | | | | | NO: | | | | |
| | | | | | 38) | | | | |
| FGFR3 | COSM4616014 | chr4: | chr4 | 1799410 | TG | T | COSM4616014 | ENSP00000339824.4: | TG > T |
| | | 1799253- | | | | | | p.Gln92SerfsTer6 | |
| | | 1799523 | | | | | | | |
| FGFR3 | COSM4992106 | chr4: | chr4 | 1803754 | TGAGG | T | COSM4992106 | | TGAGG |
| | | 1803691- | | | ACGC | | | | ACGC > T |
| | | 1803836 | | | | | | | |
| PDGFRA | COSM6906234 | chr4: | chr4 | 54264999 | GT | G | COSM6906234 | ENSP00000257290.5: | GT > G |
| | | 54264918- | | | | | | p.Phe238LeufsTer16 | |
| | | 54265049 | | | | | | | |
| PDGFRA | COSM6964190 | chr4: | chr4 | 54267667 | GCTGA | G | COSM6964190 | ENSP00000257290.5: | GCTGA |
| | | 54267551- | | | AAAAC | | | p.Lys351_ | AAAAC |
| | | 54267741 | | | AATCT | | | Leu356del | AATCT |
| | | | | | GACT | | | | GACT > G |
| | | | | | (SEQ | | | | |
| | | | | | ID | | | | |
| | | | | | NO: | | | | |
| | | | | | 39) | | | | |
| PDGFRA | COSM3301372 | chr4: | chr4 | 54285479 | CA | C | COSM3301372 | ENSP00000257290.5: | CA > C |
| | | 54285370- | | | | | | p.Asn813IlefsTer20 | |
| | | 54285486 | | | | | | | |
| PDGFRA | COSM7346029 | chr4: | chr4 | 54287473 | AC | A | COSM7346029 | ENSP00000257290.5: | AC > A |
| | | 54287429- | | | | | | p.Asp869GlufsTer7 | |
| | | 54287541 | | | | | | | |
| PDGFRA | COSM6972086 | chr4: | chr4 | 54289073 | AGT | AC | COSM6972086 | ENSP00000257290.5: | AGT > AC |
| | | 54289008- | | | | | | p.Ser947ThrfsTer24 | |
| | | 54289114 | | | | | | | |
| PDGFRA | COSM6956086 | chr4: | chr4 | 54290548 | GAC | G | COSM6956086 | ENSP00000257290.5: | GAC > G |
| | | 54290312- | | | | | | p.His 1040GlnfsTer6 | |
| | | 54290554 | | | | | | | |
| PDGFRA | COSM7346028 | chr4: | chr4 | 54295233 | CAT | C | COSM7346028 | ENSP00000257290.5: | CAT > C |
| | | 54295124- | | | | | | p.Ile1078ArgfsTer41 | |
| | | 54295272 | | | | | | | |
| KIT | COSM6951399 | chr4: | chr4 | 54658073 | TC | T | COSM6951399 | ENSP00000288135.5: | TC > T |
| | | 54658014- | | | | | | p.Gln21ArgfsTer9 | |
| | | 54658081 | | | | | | | |
| KIT | COSM7345631 | chr4: | chr4 | 54695772 | TTTG | T | COSM7345631 | ENSP00000288135.5: | TTTG > T |
| | | 54695511- | | | | | | p.Val111del | |
| | | 54695781 | | | | | | | |
| KIT | COSM7345632 | chr4: | chr4 | 54698515 | AG | A | COSM7345632 | ENSP00000288135.5: | AG > A |
| | | 54698283- | | | | | | p.Glu191ArgfsTer12 | |
| | | 54698565 | | | | | | | |
| KIT | COSM1305 | chr4: | chr4 | 54729451 | TATAA | T | COSM1305 | ENSP00000288135.5: | TATAA |
| | | 54729334- | | | GA | | | p.Lys704_ | GA > T |
| | | 54729485 | | | | | | Asn705del | |
| KIT | COSM1306 | chr4: | chr4 | 54731324 | CCAG | C | COSM1306 | ENSP00000288135.5: | CCAG > C |
| | | 54731327- | | | | | | p.Ser715del | |
| | | 54731419 | | | | | | | |
| KIT | COSM28578 | chr4: | chr4 | 54731967 | CA | C | COSM28578 | ENSP00000288135.5: | CA > C |
| | | 54731870- | | | | | | p.Lys778ArgfsTer36 | |
| | | 54731998 | | | | | | | |
| KIT | COSM6965292 | chr4: | chr4 | 54738465 | CAG | C | COSM6965292 | ENSP00000288135.5: | CAG > C |
| | | 54738428- | | | | | | p.Lys948AlafsTer100 | |
| | | 54738557 | | | | | | | |
| APC | COSM6963650 | chr5: | chr5 | 112755023 | AAGGT | A | COSM6963650 | | AAGGT |
| | | 112754890- | | | ATC | | | | ATC > A |
| | | 112755025 | | | | | | | |
| APC | COSM6853815 | chr5: | chr5 | 112766390 | AT | A | COSM6853815 | ENSP00000257430.4: | AT > A |
| | | 112766325- | | | | | | p.Leu68TyrfsTer2 | |
| | | 112766410 | | | | | | | |
| APC | COSM6854236 | chr5: | chr5 | 112775710 | AATAG | A | COSM6854236 | ENSP00000257430.4: | AATAG |
| | | 112775628- | | | | | | p.Asp170ValfsTer4 | > A |
| | | 112775737 | | | | | | | |
| APC | COSM6976104 | chr5: | chr5 | 112792468 | AAATC | A | COSM6976104 | ENSP00000257430.4: | AAATC |
| | | 112792445- | | | G | | | p.Ile224LysfsTer26 | G > A |
| | | 112792529 | | | | | | | |
| APC | COSM6984704 | chr5: | chr5 | 112801284 | ATC | A | COSM6984704 | ENSP00000257430.4: | ATC > A |
| | | 112801278- | | | | | | p.Gln247GlufsTer4 | |
| | | 112801383 | | | | | | | |
| APC | COSM6971752 | chr5: | chr5 | 112821942 | AATGA | A | COSM6971752 | ENSP00000257430.4: | AATGA |
| | | 112821895- | | | AACTT | | | p.Leu456SerfsTer6 | AACTT |
| | | 112821991 | | | TCATT | | | | TCATT |
| | | | | | TG | | | | TG > A |
| | | | | | (SEQ | | | | |
| | | | | | ID | | | | |
| | | | | | NO: | | | | |
| | | | | | 40) | | | | |
| APC | COSM4169285 | chr5: | chr5 | 112827121 | CATTG | C | COSM4169285 | ENSP00000257430.4: | CATTG |
| | | 112827107- | | | CAGAA | | | p.Glu477SerfsTer4 | CAGAA |
| | | 112827247 | | | TT | | | | TT > C |
| | | | | | (SEQ | | | | |
| | | | | | ID | | | | |
| | | | | | NO: | | | | |
| | | | | | 41) | | | | |
| APC | COSM1169625 | chr5: | chr5 | 112827937 | ATGCT | A | COSM1169625 | ENSP00000257430.4: | ATGCT |
| | | 112827928- | | | C | | | p.Cys520TyrfsTer15 | C > A |
| | | 112828006 | | | | | | | |
| APC | COSM4169180 | chr5: | chr5 | 112828863 | CGAGT | C | COSM4169180 | ENSP00000257430.4: | CGAGT |
| | | 112828855- | | | | | | p.Ser546PhefsTer2 | > C |
| | | 112828972 | | | | | | | |
| ROS1 | COSM6921151 | chr6: | chr6 | 117310082 | ATACA | A | COSM6921151 | ENSP00000357494.3: | ATACA |
| | | 117310080- | | | T | | | p.Asp2143ValfsTer25 | T > A |
| | | 117310281 | | | | | | | |
| ROS1 | COSM6959063 | chr6: | chr6 | 117326294 | TCTGA | T | COSM6959063 | ENSP00000357494.3: | TCTGA |
| | | 117326223- | | | A | | | p.Phe1828SerfsTer6 | A > T |
| | | 117326414 | | | | | | | |
| ROS1 | COSM6968834 | chr6: | chr6 | 117341465 | ATTCA | A | COSM6968834 | ENSP00000357494.3: | ATTCA |
| | | 117341399- | | | CTTTG | | | p.Thr1606_ | CTTTG |
| | | 117341632 | | | TCTTA | | | Glu1612del | TCTTA |
| | | | | | GAGGA | | | | GAGGA |
| | | | | | GT | | | | GT > A |
| | | | | | (SEQ | | | | |
| | | | | | ID | | | | |
| | | | | | NO: | | | | |
| | | | | | 42) | | | | |
| ROS1 | COSM9225153 | chr6: | chr6 | 117342404 | AT | A | COSM9225153 | ENSP00000357494.3: | AT > A |
| | | 117342399- | | | | | | p.Asn1555MetfsTer48 | |
| | | 117342544 | | | | | | | |
| ROS1 | COSM6978532 | chr6: | chr6 | 117356904 | CAATA | C | COSM6978532 | | CAATA |
| | | 117356628- | | | CAAGC | | | | CAAGC |
| | | 117356915 | | | GACTA | | | | GACTA |
| | | | | | TAGAG | | | | TAGAG |
| | | | | | GAAAA | | | | GAAAA |
| | | | | | (SEQ | | | | > C |
| | | | | | ID | | | | |
| | | | | | NO: | | | | |
| | | | | | 43) | | | | |
| ROS1 | COSM6984106 | chr6: | chr6 | 117357806 | TA | T | COSM6984106 | ENSP00000357494.3: | TA > T |
| | | 117357803- | | | | | | p.Leu1284Ter | |
| | | 117358009 | | | | | | | |
| ROS1 | COSM5977598 | chr6: | chr6 | 117360400 | CCT | C | COSM5977598 | ENSP00000357494.3: | CCT > C |
| | | 117360341- | | | | | | p.Arg1129GlyfsTer5 | |
| | | 117360405 | | | | | | | |
| ROS1 | COSM5576297 | chr6: | chr6 | 117362635 | CT | C | COSM5576297 | ENSP00000357494.3: | CT > C |
| | | 117362602- | | | | | | p.Gly1117AlafsTer2 | |
| | | 117362865 | | | | | | | |
| ROS1 | COSM7409277 | chr6: | chr6 | 117387804 | AG | A | COSM7409277 | ENSP00000357494.3: | AG > A |
| | | 117387779- | | | | | | p.Ser664GInfsTer18 | |
| | | 117388034 | | | | | | | |
| ROS1 | COSM6940064 | chr6: | chr6 | 117393245 | AT | A | COSM6940064 | ENSP00000357494.3: | AT > A |
| | | 117393223- | | | | | | p.Ile414LeufsTer14 | |
| | | 117393321 | | | | | | | |
| ROS1 | COSM6916198 | chr6: | chr6 | 117396979 | AT | A | COSM6916198 | ENSP00000357494.3: | AT > A |
| | | 117396914- | | | | | | p.Lys238AsnfsTer2 | |
| | | 117397116 | | | | | | | |
| MET | COSM6912457 | chr7: | chr7 | 116699692 | CTTCT | C | COSM6912457 | ENSP00000317272.6: | CTTCT |
| | | 116699084- | | | | | | p.Ser204IlefsTer12 | > C |
| | | 116700284 | | | | | | | |
| MET | COSM6975700 | chr7: | chr7 | 116740889 | ATTTC | A | COSM6975700 | ENSP00000317272.6: | ATTTC |
| | | 116740851- | | | CAGTC | | | p.Phe523_ | CAGTC |
| | | 116741025 | | | CTGCA | | | Ser527del | CTGCA |
| | | | | | G | | | | G > A |
| | | | | | (SEQ | | | | |
| | | | | | ID | | | | |
| | | | | | NO: | | | | |
| | | | | | 44) | | | | |
| MET | COSM6976259 | chr7: | chr7 | 116755455 | CTAGA | CG | COSM6976259 | ENSP00000317272.6: | CTAGA |
| | | 116755354- | | | GTTCT | | | p.Arg602_ | GTTCT |
| | | 116755515 | | | CCTTG | | | Ser609del | CCTTG |
| | | | | | GAAAT | | | | GAAAT |
| | | | | | GAGAG | | | | GAGAG |
| | | | | | C | | | | C > CG |
| | | | | | (SEQ | | | | |
| | | | | | ID | | | | |
| | | | | | NO: | | | | |
| | | | | | 45) | | | | |
| MET | COSM5977594 | chr7: | chr7 | 116758487 | AC | A | COSM5977594 | ENSP00000317272.6: | AC > A |
| | | 116758458- | | | | | | p.Pro712GlnfsTer13 | |
| | | 116758620 | | | | | | | |
| MET | COSM6937367 | chr7: | chr7 | 116759394 | TG | T | COSM6937367 | ENSP00000317272.6: | TG > T |
| | | 116759336- | | | | | | p.Ser776AlafsTer3 | |
| | | 116759490 | | | | | | | |
| MET | COSM6984036 | chr7: | chr7 | 116774940 | GACAT | G | COSM6984036 | ENSP00000317272.6: | GACAT |
| | | 116774880- | | | GTCCC | | | p.Asp1048_ | GTCCC |
| | | 116775111 | | | CCA | | | Ile1052delins Val | CCA > G |
| | | | | | (SEQ | | | | |
| | | | | | ID | | | | |
| | | | | | NO: | | | | |
| | | | | | 46) | | | | |
| MET | COSM1579075 | chr7: | chr7 | 116782056 | CA | C | COSM1579075 | ENSP00000317272.6: | CA > C |
| | | 116781987- | | | | | | p.Lys1217SerfsTer49 | |
| | | 116782097 | | | | | | | |
| MET | COSM7345743 | chr7: | chr7 | 116796002 | CATGT | C | COSM7345743 | ENSP00000317272.6: | CATGT |
| | | 116795886- | | | GAACG | | | p.Ala1372_ | GAACG |
| | | 116796124 | | | CTACT | | | Asn13 | CT |
| | | | | | T | | | | |
| | | | | | (SEQ | | | | |
| | | | | | ID | | | | |
| | | | | | NO: | | | | |
| | | | | | 47) | | | 76del | ACTT > C |
| EGFR | COSM6973876 | chr7: | chr7 | 55142292 | AAGGC | AC | COSM6973876 | ENSP00000275493.2: | AAGGC |
| | | 55142285- | | | ACGA | | | p.Gln32HisfsTer46 | ACGA > AC |
| | | 55142437 | | | | | | | |
| EGFR | COSM9494233 | chr7: | chr7 | 55154103 | CCCCG | C | COSM9494233 | ENSP00000275493.2: | CCCCG |
| | | 55154010- | | | AGGG | | | p.Pro281GlnfsTer15 | AGGG > C |
| | | 55154152 | | | | | | | |
| EGFR | COSM6962235 | chr7: | chr7 | 55155837 | TGTG | T | COSM6962235 | ENSP00000275493.2: | TGTG > T |
| | | 55155829- | | | | | | p.Val301del | |
| | | 55155946 | | | | | | | |
| EGFR | COSM7343128 | chr7: | chr7 | 55161538 | AG | A | COSM7343128 | ENSP00000275493.2: | AG > A |
| | | 55161498- | | | | | | p.Gly514AlafsTer54 | |
| | | 55161631 | | | | | | | |
| EGFR | COSM6196864 | chr7: | chr7 | 55165374 | TG | T | COSM6196864 | ENSP00000275493.2: | TG > T |
| | | 55165279- | | | | | | p.Val607SerfsTer98 | |
| | | 55165437 | | | | | | | |
| EGFR | COSM9110951 | chr7: | chr7 | 55170527 | GCCT | G | COSM9110951 | | GCCT > G |
| | | 55170306- | | | | | | | |
| | | 55170544 | | | | | | | |
| EGFR | COSM6909028 | chr7: | chr7 | 55173048 | TG | T | COSM6909028 | ENSP00000275493.2: | TG > T |
| | | 55172982- | | | | | | p.Ile664SerfsTer41 | |
| | | 55173124 | | | | | | | |
| GNAQ | COSM6342235 | chr9: | chr9 | 77721491 | TG | T | COSM6342235 | ENSP00000286548.4: | TG > T |
| | | 77721322- | | | | | | p.Ala304GlufsTer7 | |
| | | 77721513 | | | | | | | |
| GNAQ | COSM28414 | chr9: | chr9 | 77728594 | ATAAC | A | COSM28414 | ENSP00000286548.4: | ATAAC |
| | | 77728513- | | | CGAGG | | | p.Asn266PhefsTer4 | CGAGG |
| | | 77728667 | | | AGTT | | | | AGTT > A |
| | | | | | (SEQ | | | | |
| | | | | | ID | | | | |
| | | | | | NO: | | | | |
| | | | | | 48) | | | | |
| GNAQ | COSM7347398 | chr9: | chr9 | 77815756 | TAATT | T | COSM7347398 | ENSP00000286548.4: | TAATT |
| | | 77815615- | | | GTGCA | | | p.Ala108GlufsTer18 | GTGCA |
| | | 77815770 | | | TGAG | | | | TGAG > T |
| | | | | | (SEQ | | | | |
| | | | | | ID | | | | |
| | | | | | NO: | | | | |
| | | | | | 49) | | | | |
| GNAQ | COSM9113869 | chr9: | chr9 | 78031128 | GGCGT | G | COSM9113869 | ENSP00000286548.4: | GGCGT |
| | | 78031099- | | | CCCGC | | | p.Leu29ProfsTer24 | CCCGC |
| | | 78031235 | | | TTGTC | | | | TTGTC |
| | | | | | CCTGC | | | | CCTGC |
| | | | | | GGA | | | | GGA > G |
| | | | | | (SEQ | | | | |
| | | | | | ID | | | | |
| | | | | | NO: | | | | |
| | | | | | 50) | | | | |
| RET | COSM4989947 | chr10: | chr10 | 43100532 | C | T | COSM4989947 | ENSP00000347942.3: | C > T |
| | | 43100458- | | | | | | p.Pro49= | |
| | | 43100722 | | | | | | | |
| RET | COSM6947065 | chr10: | chr10 | 43106469 | G | A | COSM6947065 | ENSP00000347942.3: | G > A |
| | | 43106375- | | | | | | p.Gly321Arg | |
| | | 43106571 | | | | | | | |
| RET | COSM9277606 | chr10: | chr10 | 43109132 | C | T | COSM9277606 | ENSP00000347942.3: | C > T |
| | | 43109030- | | | | | | p.Leu389Phe | |
| | | 43109230 | | | | | | | |
| RET | COSM4418405 | chr10: | chr10 | 43118395 | G | T | COSM4418405 | ENSP00000347942.3: | G > T |
| | | 43118372- | | | | | | p.Leu769= | |
| | | 43118480 | | | | | | | |
| RET | COSM6945831 | chr 10: | chr10 | 43119624 | G | T | COSM6945831 | ENSP00000347942.3: | G > T |
| | | 43119530- | | | | | | p.Ser829Ile | |
| | | 43119745 | | | | | | | |
| RET | COSM3997965 | chr10: | chr10 | 43124887 | C | I | COSM3997965 | ENSP00000347942.3: | C > T |
| | | 43124882- | | | | | | p.Arg982Cys | |
| | | 43124982 | | | | | | | |
| RET | COSM6914657 | chr10: | chr10 | 43126707 | G | A | COSM6914657 | ENSP00000347942.3: | G > A |
| | | 43126574- | | | | | | p.Glu1058Lys | |
| | | 43126754 | | | | | | | |
| ATM, | COSM21325 | chr11: | chr11 | 108312465 | A | C | COSM21325 | ENSP00000278616.4: | A > C |
| C11orf65 | | 108312410- | | | | | | p.Glu1991Asp | |
| | | 108312498 | | | | | | | |
| ATM, | COSM7343670 | chr11: | chr11 | 108321330 | G | A | COSM7343670 | ENSP00000278616.4: | G > A |
| C11orf65 | | 108321300- | | | | | | p.Arg2161His | |
| | | 108321420 | | | | | | | |
| ATM, | COSM200673 | chr11: | chr11 | 108335854 | G | A | COSM200673 | ENSP00000278616.4: | G > A |
| C11orf65 | | 108335844- | | | | | | p.Asp2721Asn | |
| | | 108335961 | | | | | | | |
| AKT1 | COSM5044338 | chr14: | chr14 | 104770406 | A | T | COSM5044338 | ENSP00000451828.1: | A > T |
| | | 104770340- | | | | | | p.Cys460Ser | |
| | | 104770420 | | | | | | | |
| AKT1 | COSM6966503 | chr14: | chr14 | 104770769 | T | A | COSM6966503 | ENSP00000451828.1: | T > A |
| | | 104770744- | | | | | | p.Ile447Phe | |
| | | 104770847 | | | | | | | |
| AKT1 | COSM5020215 | chr14: | chr14 | 104772446 | G | A | COSM5020215 | ENSP00000451828.1: | G > A |
| | | 104772364- | | | | | | p.Gly393= | |
| | | 104772452 | | | | | | | |
| AKT1 | COSM6924152 | chr14: | chr14 | 104773963 | G | C | COSM6924152 | ENSP00000451828.1: | G > C |
| | | 104773911- | | | | | | p.Phe217Leu | |
| | | 104773980 | | | | | | | |
| AKT1 | COSM9102250 | chr14: | chr14 | 104775003 | C | T | COSM9102250 | ENSP00000451828.1: | C > T |
| | | 104774937- | | | | | | p.Asp190Asn | |
| | | 104775003 | | | | | | | |
| AKT1 | COSM6986817 | chr14: | chr14 | 104775763 | G | C | COSM6986817 | ENSP00000451828.1: | G > C |
| | | 104775651- | | | | | | p.Asp108Glu | |
| | | 104775799 | | | | | | | |
| ERBB2 | COSM5414789 | chr17: | chr17 | 39700281 | C | T | COSM5414789 | ENSP00000269571.4: | C > T |
| | | 39700238- | | | | | | p.Leu15Phe | |
| | | 39700311 | | | | | | | |
| ERBB2 | COSM9102609 | chr17: | chr17 | 39709385 | G | A | COSM9102609 | ENSP00000269571.4: | G > A |
| | | 39709317- | | | | | | p.Trp169Ter | |
| | | 39709452 | | | | | | | |
| ERBB2 | COSM6913537 | chr17: | chr17 | 39709822 | G | A | COSM6913537 | ENSP00000269571.4: | G > A |
| | | 39709812- | | | | | | p.Cys195Tyr | |
| | | 39709881 | | | | | | | |
| ERBB2 | COSM94225 | chr17: | chr17 | 39711955 | C | A | COSM94225 | ENSP00000269571.4: | C > A |
| | | 39711927- | | | | | | p.Ser310Tyr | |
| | | 39712047 | | | | | | | |
| ERBB2 | COSM9110847 | chr17: | chr17 | 39715294 | C | A | COSM9110847 | ENSP00000269571.4: | C > A |
| | | 39715285- | | | | | | p.Ala386Asp | |
| | | 39715359 | | | | | | | |
| ERBB2 | COSM7343981 | chr17: | chr17 | 39716431 | G | T | COSM7343981 | ENSP00000269571.4: | G > T |
| | | 39716300- | | | | | | p.Gln548His | |
| | | 39716433 | | | | | | | |
| TP53 | COSM9312241 | chr17: | chr17 | 7673240 | G | A | COSM9312241 | | G > A |
| | | 7673218- | | | | | | | |
| | | 7673266 | | | | | | | |
| GNA11 | COSM5611295 | chr19: | chr19 | 3110238 | ACC | ATT | COSM5611295 | ENSP00000078429.3: | ACC > ATT |
| | | 3110148- | | | | | | p.Thr76Ile | |
| | | 3110333 | | | | | | | |
| GNA11 | COSM6939602 | chr19: | chr19 | 3113334 | A | G | COSM6939602 | ENSP00000078429.3: | A > G |
| | | 3113329- | | | | | | p.Asn109Ser | |
| | | 3113484 | | | | | | | |
| GNAS | COSM6939725 | chr20: | chr20 | 58891769 | G | T | COSM6939725 | | G > T |
| | | 58891726- | | | | | | | |
| | | 58891865 | | | | | | | |
| GNAS | COSM6965756 | chr20: | chr20 | 58895644 | A | G | COSM6965756 | ENSP00000360141.3: | A > G |
| | | 58895611- | | | | | | p.Lys701Glu | |
| | | 58895684 | | | | | | | |
| GNAS | COSM9312081 | chr20: | chr20 | 58898948 | G | A | COSM9312081 | ENSP00000360141.3: | G > A |
| | | 58898940- | | | | | | p.Glu717Lys | |
| | | 58898985 | | | | | | | |
| GNAS | COSM3758661 | chr20: | chr20 | 58903752 | C | T | COSM3758661 | ENSP00000360141.3: | C > T |
| | | 58903671- | | | | | | p.Ile774= | |
| | | 58903791 | | | | | | | |
| GNAS | COSM6977578 | chr20: | chr20 | 58910048 | C | T | COSM6977578 | ENSP00000360141.3: | C > T |
| | | 58909950- | | | | | | p.Pro956Ser | |
| | | 58910081 | | | | | | | |
| GNAS | COSM4485625 | chr20: | chr20 | 58910387 | C | T | COSM4485625 | ENSP00000360141.3: | C > T |
| | | 58910333- | | | | | | p.Arg985Ter | |
| | | 58910401 | | | | | | | |
| GNAS | COSM6907299 | chr20: | chr20 | 58910782 | C | T | COSM6907299 | ENSP00000360141.3: | C > T |
| | | 58910682- | | | | | | p.Arg1023Cys | |
| | | 58910829 | | | | | | | |
| ALK | COSM7408659 | chr2: | chr2 | 29196823 | C | T | COSM7408659 | ENSP00000373700.3: | C > T |
| | | 29196769- | | | | | | p.Glu1371Lys | |
| | | 29196860 | | | | | | | |
| ALK | COSM6924954 | chr2: | chr2 | 29197575 | C | T | COSM6924954 | ENSP00000373700.3: | C > T |
| | | 29197541- | | | | | | p.Arg1347Gln | |
| | | 29197676 | | | | | | | |
| ALK | COSM6939221 | chr2: | chr2 | 29207181 | T | C | COSM6939221 | ENSP00000373700.3: | T > C |
| | | 29207170- | | | | | | p.Thr1310Ala | |
| | | 29207272 | | | | | | | |
| ALK | COSM28617 | chr2: | chr2 | 29214027 | C | T | COSM28617 | ENSP00000373700.3: | C > T |
| | | 29213983- | | | | | | p.Ala1234Thr | |
| | | 29214081 | | | | | | | |
| ALK | COSM6949625 | chr2: | chr2 | 29223444 | G | A | COSM6949625 | ENSP00000373700.3: | G > A |
| | | 29223341- | | | | | | p.Ser1086Leu | |
| | | 29223528 | | | | | | | |
| ALK | COSM6948461 | chr2: | chr2 | 29227060 | C | T | COSM6948461 | ENSP00000373700.3: | C > T |
| | | 29226921- | | | | | | p.Gly977Arg | |
| | | 29227074 | | | | | | | |
| ALK | COSM6908629 | chr2: | chr2 | 29227669 | C | T | COSM6908629 | ENSP00000373700.3: | C > T |
| | | 29227573- | | | | | | p.Gly940Asp | |
| | | 29227672 | | | | | | | |
| ALK | COSM148825 | chr2: | chr2 | 29232401 | A | G | COSM148825 | ENSP00000373700.3: | A > G |
| | | 29232303- | | | | | | p.GIy845= | |
| | | 29232448 | | | | | | | |
| ALK | COSM50296 | chr2: | chr2 | 29239766 | C | T | COSM50296 | ENSP00000373700.3: | C > T |
| | | 29239679- | | | | | | p.Val757Met | |
| | | 29239830 | | | | | | | |
| ALK | COSM6940013 | chr2: | chr2 | 29251115 | C | T | COSM6940013 | ENSP00000373700.3: | C > T |
| | | 29251104- | | | | | | p.Asp732Asn | |
| | | 29251267 | | | | | | | |
| ALK | COSM5019540 | chr2: | chr2 | 29275101 | G | A | COSM5019540 | ENSP00000373700.3: | G > A |
| | | 29275098- | | | | | | p.Thr680Ile | |
| | | 29275227 | | | | | | | |
| ALK | COSM6963778 | chr2: | chr2 | 29296995 | C | G | COSM6963778 | ENSP00000373700.3: | C > G |
| | | 29296887- | | | | | | p.Glu570Asp | |
| | | 29297057 | | | | | | | |
| ALK | COSM6947853 | chr2: | chr2 | 29328379 | G | T | COSM6947853 | ENSP00000373700.3: | G > T |
| | | 29328349- | | | | | | p.Ala462Asp | |
| | | 29328481 | | | | | | | |
| ALK | COSM1172867 | chr2: | chr2 | 29383830 | C | T | COSM1172867 | ENSP00000373700.3: | C > T |
| | | 29383731- | | | | | | p.Arg395His | |
| | | 29383859 | | | | | | | |
| ALK | COSM6598514 | chr2: | chr2 | 29532024 | C | T | COSM6598514 | ENSP00000373700.3: | C > T |
| | | 29531914- | | | | | | p.Val349Ile | |
| | | 29532116 | | | | | | | |
| ALK | COSM1236664 | chr2: | chr2 | 29694870 | C | T | COSM1236664 | ENSP00000373700.3: | C > T |
| | | 29694849- | | | | | | p.Arg311His | |
| | | 29695014 | | | | | | | |
| ALK | COSM4416269 | chr2: | chr2 | 29717663 | A | T | COSM4416269 | ENSP00000373700.3: | A > T |
| | | 29717577- | | | | | | p.Pro234= | |
| | | 29717697 | | | | | | | |
| PIK3CA | COSM3205605 | chr3: | chr3 | 179199822 | G | A | COSM3205605 | ENSP00000263967.3: | G > A |
| | | 179199689- | | | | | | p.Arg162Lys | |
| | | 179199899 | | | | | | | |
| PIK3CA | COSM6931303 | chr3: | chr3 | 179201476 | A | T | COSM6931303 | ENSP00000263967.3: | A > T |
| | | 179201289- | | | | | | p.Tyr250Phe | |
| | | 179201540 | | | | | | | |
| PIK3CA | COSM21450 | chr3: | chr3 | 179204576 | G | T | COSM21450 | ENSP00000263967.3: | G > T |
| | | 179204502- | | | | | | p.Cys378Phe | |
| | | 179204588 | | | | | | | |
| PIK3CA | COSM1716809 | chr3: | chr3 | 179219228 | C | T | COSM1716809 | ENSP00000263967.3: | C > T |
| | | 179219195- | | | | | | p.Pro566Leu | |
| | | 179219277 | | | | | | | |
| PIK3CA | COSM250052 | chr3: | chr3 | 179219950 | T | C | COSM250052 | ENSP00000263967.3: | T > C |
| | | 179219948- | | | | | | p.Val638Ala | |
| | | 179220052 | | | | | | | |
| PIK3CA | COSM6475729 | chr3: | chr3 | 179224123 | T | C | COSM6475729 | ENSP00000263967.3: | T > C |
| | | 179224080- | | | | | | p.Phe744Leu | |
| | | 179224187 | | | | | | | |
| PIK3CA | COSM6981846 | chr3: | chr3 | 179224740 | C | A | COSM6981846 | ENSP00000263967.3: | C > A |
| | | 179224699- | | | | | | p.Leu779Met | |
| | | 179224821 | | | | | | | |
| PIK3CA | COSM1041507 | chr3: | chr3 | 179225997 | C | T | COSM1041507 | ENSP00000263967.3: | C > T |
| | | 179225961- | | | | | | p.Arg818Cys | |
| | | 179226040 | | | | | | | |
| PIK3CA | COSM39499 | chr3: | chr3 | 179229374 | G | C | COSM39499 | ENSP00000263967.3: | G > C |
| | | 179229271- | | | | | | p.Leu866Phe | |
| | | 179229442 | | | | | | | |
| PIK3CA | COSM769 | chr3: | chr3 | 179230039 | G | T | COSM769 | ENSP00000263967.3: | G > T |
| | | 179230003- | | | | | | p.Cys901Phe | |
| | | 179230121 | | | | | | | |
| PIK3CA | COSM6475740 | chr3: | chr3 | 179230373 | A | G | COSM6475740 | ENSP00000263967.3: | A > G |
| | | 179230224- | | | | | | p.Glu978Gly | |
| | | 179230376 | | | | | | | |
| PIK3CA, | COSM9111593 | chr3: | chr3 | 179240022 | T | C | COSM9111593 | | T > C |
| KCNMB3 | | 179239995- | | | | | | | |
| | | 179240064 | | | | | | | |
| CTNNB | COSM4117539 | chr3: | chr3 | 41224075 | A | G | COSM4117539 | ENSP00000495360.1: | A > G |
| 1 | | 41224068- | | | | | | p.Thr3Ala | |
| | | 41224081 | | | | | | | |
| CTNNB | COSM5576265 | chr3: | chr3 | 41234157 | C | T | COSM5576265 | ENSP00000495360.1: | C > T |
| 1 | | 41234138- | | | | | | p.Arg515Ter | |
| | | 41234297 | | | | | | | |
| CTNNB | COSM1044608 | chr3: | chr3 | 41238068 | G | A | COSM1044608 | ENSP00000495360.1: | G > A |
| 1 | | 41238015- | | | | | | p.Arg710His | |
| | | 41238076 | | | | | | | |
| CTNNB | COSM1485172 | chr3: | chr3 | 41239208 | G | A | COSM1485172 | ENSP00000495360.1: | G > A |
| 1 | | 41239133- | | | | | | p.Glu738Lys | |
| | | 41239342 | | | | | | | |
| FGFR3 | COSM6968758 | chr4: | chr4 | 1794007 | T | A | COSM6968758 | ENSP00000339824.4: | T > A |
| | | 1793934- | | | | | | p.Leu25Met | |
| | | 1794043 | | | | | | | |
| FGFR3 | COSM9213245 | chr4: | chr4 | 1799784 | C | T | COSM9213245 | ENSP00000339824.4: | C > T |
| | | 1799746- | | | | | | p.Asp139= | |
| | | 1799812 | | | | | | | |
| FGFR3 | COSM6942045 | chr4: | chr4 | 1801370 | C | I | COSM6942045 | ENSP00000339824.4: | C > T |
| | | 1801366- | | | | | | p.Ala150Val | |
| | | 1801536 | | | | | | | |
| FGFR3 | COSM7342301 | chr4: | chr4 | 1802941 | G | A | COSM7342301 | ENSP00000339824.4: | G > A |
| | | 1802913- | | | | | | p.Asp320Asn | |
| | | 1803064 | | | | | | | |
| FGFR3 | COSM6919387 | chr4: | chr4 | 1805402 | T | C | COSM6919387 | ENSP00000339824.4: | T > C |
| | | 1805354- | | | | | | p.Val489Ala | |
| | | 1805476 | | | | | | | |
| PDGFR | COSM4383728 | chr4: | chr4 | 54258785 | C | T | COSM4383728 | ENSP00000257290.5: | C > T |
| A | | 54258768- | | | | | | p.Pro6Leu | |
| | | 54258817 | | | | | | | |
| PDGFR | COSM4416371 | chr4: | chr4 | 54263911 | T | C | COSM4416371 | ENSP00000257290.5: | T > C |
| A | | 54263666- | | | | | | p.Asn204= | |
| | | 54263927 | | | | | | | |
| PDGFR | COSM6938810 | chr4: | chr4 | 54272497 | G | A | COSM6938810 | ENSP00000257290.5: | G > A |
| A | | 54272393- | | | | | | p.Trp447Ter | |
| | | 54272520 | | | | | | | |
| PDGFR | COSM2155032 | chr4: | chr4 | 54273575 | A | G | COSM2155032 | ENSP00000257290.5: | A > G |
| A | | 54273536- | | | | | | p.Asn468Ser | |
| | | 54273730 | | | | | | | |
| PDGFR | COSM4417622 | chr4: | chr4 | 54277410 | G | A | COSM4417622 | ENSP00000257290.5: | G > A |
| A | | 54277387- | | | | | | p.Ala603= | |
| | | 54277492 | | | | | | | |
| PDGFR | COSM6958142 | chr4: | chr4 | 54278422 | A | C | COSM6958142 | ENSP00000257290.5: | A > C |
| A | | 54278361- | | | | | | p.Lys688Thr | |
| | | 54278515 | | | | | | | |
| PDGFR | COSM4383732 | chr4: | chr4 | 54280374 | A | G | COSM4383732 | ENSP00000257290.5: | A > G |
| A | | 54280315- | | | | | | p.Thr739Ala | |
| | | 54280482 | | | | | | | |
| KIT | COSM6909371 | chr4: | chr4 | 54699686 | G | T | COSM6909371 | ENSP00000288135.5: | G > T |
| | | 54699629- | | | | | | p.Gly226Trp | |
| | | 54699766 | | | | | | | |
| KIT | COSM3301432 | chr4: | chr4 | 54703760 | G | A | COSM3301432 | ENSP00000288135.5: | G > A |
| | | 54703723- | | | | | | p.Gly265Ser | |
| | | 54703892 | | | | | | | |
| KIT | COSM9500507 | chr4: | chr4 | 54707133 | A | G | COSM9500507 | ENSP00000288135.5: | A > G |
| | | 54707097- | | | | | | p.Thr321Ala | |
| | | 54707287 | | | | | | | |
| KIT | COSM6005552 | chr4: | chr4 | 54709427 | C | T | COSM6005552 | ENSP00000288135.5: | C > T |
| | | 54709423- | | | | | | p.Tyr373= | |
| | | 54709539 | | | | | | | |
| KIT | COSM1325 | chr4: | chr4 | 54736599 | G | C | COSM1325 | ENSP00000288135.5: | G > C |
| | | 54736497- | | | | | | p.Leu862= | |
| | | 54736609 | | | | | | | |
| KIT | COSM6945539 | chr4: | chr4 | 54737225 | C | A | COSM6945539 | ENSP00000288135.5: | C > A |
| | | 54737174- | | | | | | p.Thr916Lys | |
| | | 54737280 | | | | | | | |
| ROS1 | COSM249317 | chr6: | chr6 | 117288728 | C | T | COSM249317 | ENSP00000357494.3: | C > T |
| | | 117288491- | | | | | | p.Glu2270Lys | |
| | | 117288802 | | | | | | | |
| ROS1 | COSM150168 | chr6: | chr6 | 117301021 | G | C | COSM150168 | ENSP00000357494.3: | G > C |
| | | 117300973- | | | | | | p.Ser2229Cys | |
| | | 117301137 | | | | | | | |
| ROS1 | COSM5576148 | chr6: | chr6 | 117308866 | G | T | COSM5576148 | ENSP00000357494.3: | G > T |
| | | 117308793- | | | | | | p.Ser2166Tyr | |
| | | 117308928 | | | | | | | |
| ROS1 | COSM6950684 | chr6: | chr6 | 117311094 | C | A | COSM6950684 | ENSP00000357494.3: | C > A |
| | | 117311019- | | | | | | p.Leu2053Phe | |
| | | 117311117 | | | | | | | |
| ROS1 | COSM6950893 | chr6: | chr6 | 117318188 | C | T | COSM6950893 | ENSP00000357494.3: | C > T |
| | | 117318187- | | | | | | p.Ser2002Asn | |
| | | 117318252 | | | | | | | |
| ROS1 | COSM9513198 | chr6: | chr6 | 117321391 | C | A | COSM9513198 | ENSP00000357494.3: | C > A |
| | | 117321258- | | | | | | p.Trp1882Leu | |
| | | 117321394 | | | | | | | |
| ROS1 | COSM6941244 | chr6: | chr6 | 117324337 | G | A | COSM6941244 | ENSP00000357494.3: | G > A |
| | | 117324331- | | | | | | p.Thr1879Ile | |
| | | 117324415 | | | | | | | |
| ROS1 | COSM6965416 | chr6: | chr6 | 117329359 | C | A | COSM6965416 | ENSP00000357494.3: | C > A |
| | | 117329328- | | | | | | p.Cys1779Phe | |
| | | 117329446 | | | | | | | |
| ROS1 | COSM9125580 | chr6: | chr6 | 117337327 | T | C | COSM9125580 | ENSP00000357494.3: | T > C |
| | | 117337171- | | | | | | p.Glu1698Gly | |
| | | 117337340 | | | | | | | |
| ROS1 | COSM6969339 | chr6: | chr6 | 117344154 | C | T | COSM6969339 | ENSP00000357494.3: | C > T |
| | | 117344059- | | | | | | p.Trp1477Ter | |
| | | 117344262 | | | | | | | |
| ROS1 | COSM4992412 | chr6: | chr6 | 117353036 | C | T | COSM4992412 | ENSP00000357494.3: | C > T |
| | | 117352989- | | | | | | p.Arg1425= | |
| | | 117353169 | | | | | | | |
| ROS1 | COSM6951463 | chr6: | chr6 | 117365132 | G | C | COSM6951463 | ENSP00000357494.3: | G > C |
| | | 117365059- | | | | | | p.Leu1016Val | |
| | | 117365204 | | | | | | | |
| ROS1 | COSM6954777 | chr6: | chr6 | 117365633 | G | A | COSM6954777 | ENSP00000357494.3: | G > A |
| | | 117365580- | | | | | | p.Ala974Val | |
| | | 117365741 | | | | | | | |
| ROS1 | COSM95208 | chr6: | chr6 | 117366216 | T | C | COSM95208 | ENSP00000357494.3: | T > C |
| | | 117366075- | | | | | | p.Tyr891Cys | |
| | | 117366290 | | | | | | | |
| ROS1 | COSM4992418 | chr6: | chr6 | 117379095 | C | T | COSM4992418 | ENSP00000357494.3: | C > T |
| | | 117379058- | | | | | | p.Cys854Tyr | |
| | | 117379159 | | | | | | | |
| ROS1 | COSM7342701 | chr6: | chr6 | 117383402 | G | T | COSM7342701 | ENSP00000357494.3: | G > T |
| | | 117383316- | | | | | | p.Thr804Asn | |
| | | 117383508 | | | | | | | |
| ROS1 | COSM9277478 | chr6: | chr6 | 117385755 | G | C | COSM9277478 | ENSP00000357494.3: | G > C |
| | | 117385682- | | | | | | p.Ser744Arg | |
| | | 117385861 | | | | | | | |
| ROS1 | COSM6977455 | chr6: | chr6 | 117386990 | G | A | COSM6977455 | ENSP00000357494.3: | G > A |
| | | 117386888- | | | | | | p.Pro675Leu | |
| | | 117386999 | | | | | | | |
| ROS1 | COSM6912204 | chr6: | chr6 | 117389389 | G | C | COSM6912204 | ENSP00000357494.3: | G > C |
| | | 117389349- | | | | | | p.Leu574Val | |
| | | 117389846 | | | | | | | |
| ROS1 | COSM6912725 | chr6: | chr6 | 117394313 | T | A | COSM6912725 | ENSP00000357494.3: | T > A |
| | | 117394161- | | | | | | p.Tyr338Phe | |
| | | 117394346 | | | | | | | |
| ROS1 | COSM6968764 | chr6: | chr6 | 117394705 | C | T | COSM6968764 | ENSP00000357494.3: | C > T |
| | | 117394615- | | | | | | p.Arg297Lys | |
| | | 117394738 | | | | | | | |
| ROS1 | COSM6921289 | chr6: | chr6 | 117396196 | G | C | COSM6921289 | ENSP00000357494.3: | G > C |
| | | 117396187- | | | | | | p.Ser283Cys | |
| | | 117396264 | | | | | | | |
| ROS1 | COSM5019315 | chr6: | chr6 | 117403216 | C | T | COSM5019315 | ENSP00000357494.3: | C > T |
| | | 117403138- | | | | | | p.Arg167Gln | |
| | | 117403277 | | | | | | | |
| ROS1 | COSM3761460 | chr6: | chr6 | 117404415 | T | A | COSM3761460 | ENSP00000357494.3: | T > A |
| | | 117404279- | | | | | | p.Leu101= | |
| | | 117404428 | | | | | | | |
| ROS1 | COSM6952051 | chr6: | chr6 | 117409597 | C | T | COSM6952051 | ENSP00000357494.3: | C > T |
| | | 117409581- | | | | | | p.Glu92Lys | |
| | | 117409642 | | | | | | | |
| ROS1 | COSM4604501 | chr6: | chr6 | 117416316 | C | T | COSM4604501 | ENSP00000357494.3: | C > T |
| | | 117416257- | | | | | | p.Cys57Tyr | |
| | | 117416317 | | | | | | | |
| ROS1 | COSM6355496 | chr6: | chr6 | 117418506 | C | T | COSM6355496 | ENSP00000357494.3: | C > T |
| | | 117418461- | | | | | | p.Gly42Ser | |
| | | 117418506 | | | | | | | |
| ROS1 | COSM6910632 | chr6: | chr6 | 117425580 | T | C | COSM6910632 | ENSP00000357494.3: | T > C |
| | | 117425533- | | | | | | p.Gln26Arg | |
| | | 117425656 | | | | | | | |
| MET | COSM5945634 | chr7: | chr7 | 116731717 | G | A | COSM5945634 | ENSP00000317272.6: | G > A |
| | | 116731667- | | | | | | p.Arg417Gln | |
| | | 116731859 | | | | | | | |
| MET | COSM6927005 | chr7: | chr7 | 116739966 | C | T | COSM6927005 | ENSP00000317272.6: | C > T |
| | | 116739949- | | | | | | p.Ser470Leu | |
| | | 116740084 | | | | | | | |
| MET | COSM3632213 | chr7: | chr7 | 116757727 | G | A | COSM3632213 | ENSP00000317272.6: | G > A |
| | | 116757637- | | | | | | p.Gly685= | |
| | | 116757774 | | | | | | | |
| MET | COSM5047343 | chr7: | chr7 | 116769789 | G | C | COSM5047343 | ENSP00000317272.6: | G > C |
| | | 116769644- | | | | | | p.Glu928Gln | |
| | | 116769791 | | | | | | | |
| MET | COSM5609378 | chr7: | chr7 | 116771624 | C | T | COSM5609378 | ENSP00000317272.6: | C > T |
| | | 116771497- | | | | | | p.Leu971= | |
| | | 116771654 | | | | | | | |
| MET | COSM6438054 | chr7: | chr7 | 116777440 | A | T | COSM6438054 | ENSP00000317272.6: | A > T |
| | | 116777388- | | | | | | p.Lys1122Ile | |
| | | 116777469 | | | | | | | |
| MET | COSM6983877 | chr7: | chr7 | 116778811 | ACC | ATT | COSM6983877 | ENSP00000317272.6: | ACC > ATT |
| | | 116778775- | | | | | | p.Thr1144Ile | |
| | | 116778957 | | | | | | | |
| EGFR | COSM6937748 | chr7: | chr7 | 55019353 | G | A | COSM6937748 | ENSP00000275493.2: | G > A |
| | | 55019277- | | | | | | p.Glu26Lys | |
| | | 55019365 | | | | | | | |
| EGFR | COSM9233361 | chr7: | chr7 | 55143416 | G | T | COSM9233361 | ENSP00000275493.2: | G > T |
| | | 55143304- | | | | | | p.Ala118Ser | |
| | | 55143488 | | | | | | | |
| EGFR | COSM42978 | chr7: | chr7 | 55146655 | C | T | COSM42978 | ENSP00000275493.2: | C > T |
| | | 55146605- | | | | | | p.Asn158= | |
| | | 55146740 | | | | | | | |
| EGFR | COSM7002280 | chr7: | chr7 | 55151308 | C | G | COSM7002280 | ENSP00000275493.2: | C > G |
| | | 55151293- | | | | | | p.Pro192Ala | |
| | | 55151362 | | | | | | | |
| EGFR | COSM4166393 | chr7: | chr7 | 55152627 | C | T | COSM4166393 | ENSP00000275493.2: | C > T |
| | | 55152545- | | | | | | p.Ala237Val | |
| | | 55152664 | | | | | | | |
| EGFR | COSM6970489 | chr7: | chr7 | 55156802 | G | A | COSM6970489 | ENSP00000275493.2: | G > A |
| | | 55156758- | | | | | | p.Asp393Asn | |
| | | 55156843 | | | | | | | |
| EGFR | COSM7002279 | chr7: | chr7 | 55157735 | G | A | COSM7002279 | ENSP00000275493.2: | G > A |
| | | 55157662- | | | | | | p.Arg427His | |
| | | 55157753 | | | | | | | |
| EGFR | COSM236670 | chr7: | chr7 | 55160316 | C | A | COSM236670 | ENSP00000275493.2: | C > A |
| | | 55160138- | | | | | | p.Ser492Arg | |
| | | 55160338 | | | | | | | |
| EGFR | COSM5530405 | chr7: | chr7 | 55163734 | G | A | COSM5530405 | ENSP00000275493.2: | G > A |
| | | 55163732- | | | | | | p.Glu545Lys | |
| | | 55163823 | | | | | | | |
| EGFR | COSM3762772 | chr7: | chr7 | 55171181 | T | A | COSM3762772 | ENSP00000275493.2: | T > A |
| | | 55171174- | | | | | | p.Thr629= | |
| | | 55171213 | | | | | | | |
| EGFR | COSM6976991 | chr7: | chr | 55192784 | G | A | COSM6976991 | ENSP00000275493.2: | G > A |
| | | 55192765- | | | | | | p.Ala882Thr | |
| | | 55192841 | | | | | | | |
| EGFR | COSM6932208 | chr7: | chr7 | 55198789 | C | T | COSM6932208 | ENSP00000275493.2: | C > T |
| | | 55198716- | | | | | | p.Ser925Phe | |
| | | 55198863 | | | | | | | |
| EGFR | COSM5762244 | chr7: | chr7 | 55200351 | C | T | COSM5762244 | ENSP00000275493.2: | C > T |
| | | 55200315- | | | | | | p.Arg962Cys | |
| | | 55200413 | | | | | | | |
| EGFR | COSM3762773 | chr7: | chr7 | 55201223 | C | T | COSM3762773 | ENSP00000275493.2: | C > T |
| | | 55201187- | | | | | | p.Asp994= | |
| | | 55201355 | | | | | | | |
| EGFR | COSM6925302 | chr7: | chr7 | 55201765 | T | C | COSM6925302 | ENSP00000275493.2: | T > C |
| | | 55201734- | | | | | | p.Cys1049Arg | |
| | | 55201782 | | | | | | | |
| EGFR | COSM7410173 | chr7: | chr7 | 55202527 | G | A | COSM7410173 | ENSP00000275493.2: | G > A |
| | | 55202516- | | | | | | p.Cys1058Tyr | |
| | | 55202625 | | | | | | | |
| EGFR | COSM9496259 | chr7: | chr7 | 55205525 | G | A | COSM9496259 | ENSP00000275493.2: | G > A |
| | | 55205255- | | | | | | p.Ala1181Thr | |
| | | 55205617 | | | | | | | |
| GNAQ | COSM52975 | chr9: | chr9 | 77797577 | C | T | COSM52975 | ENSP00000286548.4: | C > T |
| | | 77797519- | | | | | | p.Arg183Gln | |
| | | 77797648 | | | | | | | |

In some instances, the variant sequence is a variant described in Table 3, below.

TABLE 3

| fusion_name | chrom_5 | pos_5 | chrom_3 | pos_3 | genome_build |
|---|---|---|---|---|---|
| TPR-ALK | chr1 | 186,356,039 | chr2 | 29,224,077 | hg38 |
| NCOA4-RET | chr10 | 46,011,368 | chr10 | 43,116,070 | hg38 |
| EML4-ALK_1 | chr2 | 42296684 | chr2 | 29223819 | hg38 |
| EML4-ALK_2 | chr2 | 42274039 | chr2 | 29225364 | hg38 |
| EML4-ALK_3 | chr2 | 42299217 | chr2 | 29224971 | hg38 |
| KIF5B-RET_1 | chr10 | 32024672 | chr10 | 43115128 | hg38 |
| KIF5B-RET_2 | chr10 | 32017899 | chr10 | 43116571 | hg38 |
| KIF5B-RET_3 | chr10 | 32016770 | chr10 | 43111682 | hg38 |
| CDC6-RET_1 | chr10 | 59902926 | chr10 | 43116231 | hg38 |
| CDC6-RET_2 | chr10 | 59856493 | chr10 | 43115739 | hg38 |
| CDC6-RET_3 | chr10 | 59878856 | chr10 | 43114499 | hg38 |
| TMPRSS2-ERG_1 | chr21 | 41500529 | chr21 | 38459804 | hg38 |
| TMPRSS2-ERG_2 | chr21 | 41498978 | chr21 | 38498963 | hg38 |
| TMPRSS2-ERG_3 | chr21 | 41492789 | chr21 | 38454919 | hg38 |
| TMPRSS2-ERG_4 | chr21 | 41491740 | chr21 | 38504508 | hg38 |

In some instances, the variant sequence is a variant described in Table 4, below.

TABLE 4

| | | | | | | | | Cosmic_ | ddPCR_ | Var_ |
|---|---|---|---|---|---|---|---|---|---|---|
| Chrom | Start | End | Gene | Protein | DNA | Ref | Alt | id | quant | length |
| 1 | 114713907 | 114713908 | NRAS/ | p.Q61R | c.182A > G | T | C | COSM | 0.0221 | 1 |
| | | | CSDE1 | | | | | 584 | | |
| 3 | 179234360 | 179234361 | PIK3CA | p.N1068fs*4 | c.3204_ | C | C +1A | COSM | 0.0187 | 1 |
| | | | | | 3205insA | | | 12464 | | |
| 4 | 54274880 | 54274881 | PDGFRA | p.S566fs*6 | c. 1694_ | T | T +1A | COSM | 0.0224 | 1 |
| | | | | | 1695insA | | | 28053 | | |
| 5 | 112840253 | 112840254 | APC | p.T1556fs*3 | c.4666_ | G | G +1A | COSM | 0.0181 | 1 |
| | | | | | 4667insA | | | 18561 | | |
| 10 | 87957957 | 87957958 | PTEN | p.P248fs*5 | c.741_ | T | T +1A | COSM | 0.0143 | 1 |
| | | | | | 742insA | | | 4986 | | |
| 10 | 87958012 | 87958013 | PTEN | p.K267fs*9 | c.800delA | A | * | COSM | 0.0143 | 1 |
| | | | | | | | | 5809 | | |
| 11 | 108247120 | 108247121 | ATM | p.C353fS*5 | c.1058_ | | | COSM | 0.025 | 1 |
| | | | | | 1059delGT | | | 21924 | | |
| 17 | 7674219 | 7674220 | TP53 | p.R2480 | c.743G > A | C | T | COSM | 0.0204 | 1 |
| | | | | | | | | 10662 | | |
| 17 | 7674239 | 7674240 | TP53 | p.C242fS*5 | c.723delC | G | * | COSM | 0.0202 | 1 |
| | | | | | | | | 6530 | | |
| 17 | 7676101 | 7676102 | TP53 | p.S90fs*33 | c.263delC | G | * | COSM | NaN | 1 |
| | | | | | | | | 18610 | | |
| 18 | 51076721 | 51076722 | SMAD4 | p.A466fs*28 | c. 1394_ | G | G +1T | COSM | 0.0197 | 1 |
| | | | | | 1395insT | | | 14105 | | |
| 1 | 43349337 | 43349338 | MPL | p.W515L | c.1544G > T | G | T | COSM | 0.0219 | 1 |
| | | | | | | | | 18918 | | |
| 2 | 208248388 | 208248389 | IDH1 | p.R132C | c.394C > T | G | A | COSM | 0.0253 | 1 |
| | | | | | | | | 28747 | | |
| 3 | 41224632 | 41224633 | CTNNB1 | p.T41A | c. 121A > G | A | G | COSM | 0.0226 | 1 |
| | | | | | | | | 5664 | | |
| 3 | 138946320 | 138946321 | FOXL2 | p.C134W | c.402C > G | G | C | COSM | 0.0189 | 1 |
| | | | | | | | | 33661 | | |
| 3 | 179218302 | 179218303 | PIK3CA | p.E545K | c.1633G > A | G | A | COSM | 0.0177 | 1 |
| | | | | | | | | 763 | | |
| 3 | 179234296 | 179234297 | PIK3CA | p.H1047R | c.3140A > G | A | G | COSM | 0.0204 | 1 |
| | | | | | | | | 775 | | |
| 4 | 1801840 | 1801841 | FGFR3 | p.S249C | c.746C > G | C | G | COSM | 0.0199 | 1 |
| | | | | | | | | 715 | | |
| 4 | 54285925 | 54285926 | PDGFRA | p.D842V | c.2525A > T | A | T | COSM | 0.0211 | 1 |
| | | | | | | | | 736 | | |
| 4 | 54733154 | 54733155 | KIT | p.D816V | c.2447A > T | A | T | COSM | 0.0223 | 1 |
| | | | | | | | | 1314 | | |
| 5 | 112839941 | 112839942 | APC | p.R1450* | c.4348C > T | C | T | COSM | 0.0175 | 1 |
| | | | | | | | | 13127 | | |
| 5 | 171410538 | 171410539 | NPM1 | p.W288fs*12 | c.863_ | C | C +4TCTG | COSM | 0.015 | 1 |
| | | | | | 864insTCTG | | | 17559 | | |
| 7 | 55174772 | 55174787 | EGFR | p.E746_ | c.2236_ | | | COSM | 0.0243 | 15 |
| | | | | A750de | 2250del15 | | | 6225 | | |
| | | | | IELREA | | | | | | |
| 7 | 55181318 | 55181319 | EGFR | p.D770_ | c.2310_ | C | C +3GGT | COSM | 0.0214 | 1 |
| | | | | N771insG | 2311insGGT | | | 12378 | | |
| 7 | 55181377 | 55181378 | EGFR | p.T790M | c.2369C > T | C | T | COSM | 0.0214 | 1 |
| | | | | | | | | 6240 | | |
| 7 | 55191821 | 55191822 | EGFR | p.L858R | c.2573T > G | T | G | COSM | 0.0261 | 1 |
| | | | | | | | | 6224 | | |
| 7 | 140753335 | 140753336 | BRAF | p.V600E | c. 1799T > A | A | T | COSM | 0.0213 | 1 |
| | | | | | | | | 476 | | |
| 9 | 5073769 | 5073770 | JAK2 | p.V617F | c.1849G > T | G | T | COSM | 0.0198 | 1 |
| | | | | | | | | 12600 | | |
| 9 | 77794571 | 77794572 | GNAQ | p.Q209P | c.626A > C | T | G | COSM | 0.0193 | 1 |
| | | | | | | | | 28758 | | |
| 10 | 43121967 | 43121968 | RET | p.M918T | c.2753T > C | T | C | COSM | 0.0204 | 1 |
| | | | | | | | | 965 | | |
| 12 | 25245349 | 25245350 | KRAS | p.G12D | c.35G > A | C | T | COSM | 0.0203 | 1 |
| | | | | | | | | 521 | | |
| 13 | 28018504 | 28018505 | FLT3 | p.D835Y | c.2503G > T | C | A | COSM | 0.021 | 1 |
| | | | | | | | | 783 | | |
| 14 | 104780213 | 104780214 | AKT1 | p.E17K | c.49G > A | C | T | COSM | 0.022 | 1 |
| | | | | | | | | 33765 | | |
| 17 | 7673801 | 7673802 | TP53 | p.R273H | c.818G > A | C | T | COSM | 0.0196 | 1 |
| | | | | | | | | 10660 | | |
| 17 | 7675087 | 7675088 | TP53 | p.R175H | c.524G > A | C | T | COSM | 0.0209 | 1 |
| | | | | | | | | 10648 | | |
| 17 | 39724727 | 39724728 | ERBB2 | p.A775_ | c.2324_ | A | A +12G | COSM | 0.0227 | 1 |
| | | | | G776ins | 2325ins12 | | CATAC | 682/ | | |
| | | | | YVMA | | | GTGAT | 20959 | | |
| | | | | | | | G | | | |
| | | | | | | | (SEQ | | | |
| | | | | | | | ID | | | |
| | | | | | | | NO: | | | |
| | | | | | | | 51) | | | |
| 20 | 58854052 | 58854053 | GNAS | p.R201C | c.601C > T | C | T | COSM | 0.0206 | 1 |
| | | | | | | | | 27887 | | |

In some instances, the variant sequence is a variant described in Table 5, below.

TABLE 5

| Chromosome | Gene | Mutation |
|---|---|---|
| 7q34 | BRAF | V600E |
| 4q11-q12 | cKIT | D816V |
| 7p12 | EGFR | ΔE746 - A750 |
| 7p12 | EGFR | L858R |
| 7p12 | EGFR | T790M |
| 7p12 | EGFR | G719S |
| 12p12.1 | KRAS | G13D |
| 12p12.1 | KRAS | G12D |
| 1p13.2 | NRAS | Q61K |
| 3q26.3 | PIK3CA | h2047R |
| 3q26.3 | PIK3CA | E545K |
| p23 | ALK | P1543S |
| 1q25.2 | ABL2 | P986fs |
| 5q21-q22 | APC | R2714C |
| 1p35.3 | ARID1A | p.M1564fs*1 |
| 13q12.3 | BRCA2 | A1689fs |
| 13q12.3 | CDX2 | V306fs |
| 22q13.2 | EP300 | K291fs |
| 4q31.3 | FBXW7 | G667fs |
| 8p12 | FGFR1 | P150L |
| 13q12 | FLT3 | V197A |
| 2q33.3 | IDh2 | S261L |
| 7q31 | MET | V237fs |
| 3p21.3 | MLh2 | L323M |
| 17q11.2 | NF1 | L626fs |
| 22q12.2 | NF2 | P275fs |
| 9q34.3 | NOTCh2 | P668S |
| 1q21-q22 | NTRK1 | 5′UTR |
| 4q12 | PDGFRA | G426D |

In some instances, the variant sequence is a variant described in Table 6, below.

TABLE 6

| Gene | Variant Description |
|---|---|
| NRAS | G12D |
| NRAS | Q61H |
| IDH2 | R172K |
| IDH2 | R140Q |
| CTNNB1 | G34E |
| FOXL2 | — |
| PIK3CA | N345K |
| FGFR1 | N546K |
| FGFR1 | K656E |
| FGFR2 | S252W |
| FGFR2 | N549K |
| FGFR2 | C382R |
| FGFR2 | K659E |
| FGFR3 | Y373C |
| FGFR3 | K650E |
| PDGFRA | V561D |
| PDGFRA | N659K |
| PDGFRA | SPDGHE566- |
| KIT | L576P |
| KIT | V560G |
| KIT | del547-555 |
| KIT | K642E |
| EGFR | S768I |
| EGFR | G724S |
| EGFR | L792H |
| EGFR | L718Q |
| BRAF | None |
| JAK2 | None |
| GNAQ | T96S |
| RET | None |
| PTEN | None |
| KRAS | Q61H |
| FLT3 | None |
| AKT1 | L52R |
| AKT1 | Q79K |
| TP53 | G245C |
| TP53 | R282W |
| ERBB2 | L755S |
| ERBB2 | P780_Y781insGSP |
| ERBB2 | V842I |
| SMAD4 | R361H |
| GNA11 | None |
| GNAS1 | None |
| ATM | None |
| ALK | G1128A, F1174L, R1192P, R1275Q |
| AR | T878A, W742C, structural variants |
| ARAF | S214C |
| BRCA1 | c.4964_4982del19 - p.(Ser1655Tyrfs*16)/5, |
| | c.5266dupC - p.(Gln1756Profs*74)/3, |
| BRCA2 | c.5351dupA - p.(Asn1784Lysfs*3)/4 |
| CCND1 | E275*fs, T286I |
| CDH1 | R732Q, A634V |
| CDK12 | W719*, E928fs27* |
| CDK4 | R24C |
| CDK6 | — |
| CDKN2A | — |
| DDR2 | I638F, L239R |
| ESR1 | D538G |
| EZH2 | Y641F |
| FGFR2 | S252W, N550K |
| HRAS | G12V |
| JAK3 | — |
| MAP2K1 | K57 N |
| MAP2K2 | — |
| MET | d1246n |
| MTOR | — |
| NF1 | — |
| NTRK1 | Fusions |
| PTPN11 | E76K, G503R |
| RAF1 | — |
| RB1 | — |
| ROS1 | G2032R |
| SMO | D473H |
| STK11 | — |
| TERT | C228T and C250T in promoter |
| ABL1 | F317V |
| ARID1A | Q1401* |
| ATR | — |
| BAP1 | W196* |
| CCND2 | None |
| CCNE1 | None |
| CD274 | None |
| CHD1 | Q23* |
| CHEK2 | None |
| CRKL | None |
| ERBB3 | v104m |
| ERRFI1 | None |
| FBXW7 | R465C |
| FGFR4 | None |
| FH | None |
| FOXA1 | R219S |
| GATA3 | P408fs |
| HNF1A | P289fs |
| KDM5C | S1222P |
| KDM6A | p.I598fsX6 |
| MAPK1 | E322K |
| MAPK3 | None |
| MLH1 | R498fs |
| MYC | T58A |
| MYCN | P44L |
| MYD88 | L265P |
| NF2 | None |
| NFE2L2 | G333C |
| NOTCH1 | LOF frameshift mutations, E124*, W1843* |
| NTRK3 | None |
| PALB2 | — |
| PBRM1 | p.F116fs*7 |
| PDCD1LG2 | None |
| PDGFRB | None |
| RHEB | Y35N |
| RHOA | Y42C |
| RIT1 | M90I |
| SETD2 | — |
| SF3B1 | None |
| SMARCB1 | None |
| SPOP | F133V |
| TSC1 | p.Q794 |
| VEGFA | — |
| VHL | None |
| ZNF703 | None |

In some instances, the variant sequence is a variant described in any one of Tables 1-6, above.

Variant sequences (e.g., genomic variants) may be detected from a sample (e.g., genomic sample) with varying degrees of recall and precision. In some instances, the upper limit on detection is determined by performance of a reference standard described herein. In some instances, reference standards have pre-selected variant frequencies for comparison to patient samples. In some instances, recall represents the number of variant sequences detected out of all that variants expected to be detectable. In some instances, precision represents the number of variant sequences that are called correctly out of everything detected as a variant. In some instances, the variant sequence is detected with a recall of at least 30%, 50%, 75%, 80%, 85%, 90%, 95%, 97%, 98%, or at least 99%. In some instances, the variant sequence is detected with a recall of about 30%, 50%, 75%, 80%, 85%, 90%, 95%, 97%, 98%, or about 99%. In some instances, the variant sequence is detected with a recall of about 10%-99%, 25-99%, 30-90%, 45-80%, 50-99%, 75-99%, or 90-99%. In some instances, the variant sequence is detected with a precision of at least 30%, 50%, 75%, 80%, 85%, 90%, 95%, 97%, 98%, or at least 99%. In some instances, the variant sequence is detected with a precision of about 30%, 50%, 75%, 80%, 85%, 90%, 95%, 97%, 98%, or about 99%. In some instances, the variant sequence is detected with a precision of about 10%-99%, 25-99%, 30-90%, 45-80%, 50-99%, 75-99%, or 90-99%.

Polynucleotide libraries may be designed to comprise sequences which are identical to or complementary (to target, hybridize) to one or more variant sequences. In some instances, at least some of the polynucleotides are each configured to hybridize to genomic regions which comprise at least two variant sequences. In some instances, at least some of the polynucleotides are each configured to hybridize to genomic regions which comprise at least one, two, three, four, five, six, or more than six variant sequences. In some instances, at least some of the polynucleotides are each configured to hybridize to genomic regions which comprise one to four variant sequences. In some instances, at least some of the polynucleotides are each configured to hybridize to genomic regions which comprise one to two or three variant sequences. In some instances, at least 50% of the polynucleotides are each configured to hybridize to genomic regions which comprise at least two variant sequences. In some instances, at least 50% of the polynucleotides are each configured to hybridize to genomic regions which comprise at least one, two, three, four, five, six, or more than six variant sequences. In some instances, at least 50% of the polynucleotides are each configured to hybridize to genomic regions which comprise one to four variant sequences. In some instances, at least 50% of the polynucleotides are each configured to hybridize to genomic regions which comprise one to two or three variant sequences. In some instances, at least 25% of the polynucleotides are each configured to hybridize to genomic regions which comprise at least two variant sequences. In some instances, at least 25% of the polynucleotides are each configured to hybridize to genomic regions which comprise at least one, two, three, four, five, six, or more than six variant sequences. In some instances, at least 25% of the polynucleotides are each configured to hybridize to genomic regions which comprise one to four variant sequences. In some instances, at least 25% of the polynucleotides are each configured to hybridize to genomic regions which comprise one to two or three variant sequences. In some instances, at least 5% of the polynucleotides are each configured to hybridize to genomic regions which comprise at least two variant sequences. In some instances, at least 5% of the polynucleotides are each configured to hybridize to genomic regions which comprise at least one, two, three, four, five, six, or more than six variant sequences. In some instances, at least 5% of the polynucleotides are each configured to hybridize to genomic regions which comprise one to four variant sequences. In some instances, at least 5% of the polynucleotides are each configured to hybridize to genomic regions which comprise one to two or three variant sequences.

Polynucleotide libraries may be configured to bind to many variant sequences. In some instances, a polynucleotide library is collectively configured to bind to genomic regions comprising about 50, 100, 200, 500, 800, 1000, 2000, 5000, 8000, 10,000, 20,000, 50,000, 80,000, 100,000, 250,000, 500,000, 750,000, 1 million, 1.5 million, 2 million, 2.5 million, 3 million, 3.5 million, 4 million, 4.5 million, or about 5 million variant sequences. In some instances, a polynucleotide library is collectively configured to bind to genomic regions comprising at least 50, 100, 200, 500, 800, 1000, 2000, 5000, 8000, 10,000, 20,000, 50,000, 80,000, 100,000, 250,000, 500,000, 750,000, 1 million, 1.5 million, 2 million, 2.5 million, 3 million, 3.5 million, 4 million, 4.5 million, or at least 5 million variant sequences. In some instances, a polynucleotide library is collectively configured to bind to genomic regions comprising 100-1000, 50-100, 50-500, 50-5000, 50-10,000, 100,000-5 million, 250,000-3 million, 500,000-2 million, 750,000-4 million, 1 million-5 million, 1 million-3 million, 1 million-4 million, or 4 million to 6 million variant sequences.

Polynucleotide libraries for identifying variant sequences may be optimized. In some instances, the library is uniform (each unique polynucleotide is equally represented). In some instances, the library is not uniform. In some instances, polynucleotides are represented in an amount within at least about 1.5 times the mean representation for the polynucleotide library. In some instances, polynucleotides are represented in an amount within at least about 2 times the mean representation for the polynucleotide library. In some instances, polynucleotides are represented in an amount within at least about 1.2 times the mean representation for the polynucleotide library. In some instances, polynucleotides are represented in an amount within at least about 1.7 times the mean representation for the polynucleotide library. In some instances, at least 80% polynucleotides are represented in an amount within at least about 1.5 times the mean representation for the polynucleotide library. In some instances, at least 80% polynucleotides are represented in an amount within at least about 2 times the mean representation for the polynucleotide library. In some instances, at least 80% polynucleotides are represented in an amount within at least about 1.7 times the mean representation for the polynucleotide library. In some instances, at least 80% polynucleotides are represented in an amount within at least about 2 times the mean representation for the polynucleotide library. In some instances, at least 90% polynucleotides are represented in an amount within at least about 1.5 times the mean representation for the polynucleotide library. In some instances, at least 90% polynucleotides are represented in an amount within at least about 2 times the mean representation for the polynucleotide library. In some instances, at least 80% polynucleotides are represented in an amount within at least about 1.7 times the mean representation for the polynucleotide library. In some instances, at least 90% polynucleotides are represented in an amount within at least about 2 times the mean representation for the polynucleotide library. In some instances, at least 95% polynucleotides are represented in an amount within at least about 1.5 times the mean representation for the polynucleotide library. In some instances, at least 95% polynucleotides are represented in an amount within at least about 2 times the mean representation for the polynucleotide library. In some instances, at least 95% polynucleotides are represented in an amount within at least about 1.7 times the mean representation for the polynucleotide library. In some instances, at least 95% polynucleotides are represented in an amount within at least about 2 times the mean representation for the polynucleotide library. Polynucleotide libraries in some instances comprise at least some polynucleotides which each comprise an overlap region with another polynucleotide in the library. In some instances at least 10%, 20%, 30%, 40%, 50%, 60%, 70%, 80%, or at least 90% of the polynucleotides each comprise an overlap region with another polynucleotide in the library. In some instances about 10%, 20%, 30%, 40%, 50%, 60%, 70%, 80%, or about 90% of the polynucleotides each comprise an overlap region with another polynucleotide in the library. In some instances 10%-90%, 10-80%, 10-75%, 25%-50%, 25-90%, 50-90%, 15-35%, or 80-99% of the polynucleotides each comprise an overlap region with another polynucleotide in the library. In some instances, the amount of at least some of the polynucleotides in the library is 5, 10, 20, 25, 50, 75, 100, 150, 200, 250, 300, 400, 500, or 600 times higher than the mean representation for the polynucleotide library. In some instances, the amount of at least 1% of the polynucleotides in the library is 5, 10, 20, 25, 50, 75, 100, 150, 200, 250, 300, 400, 500, or 600 times higher than the mean representation for the polynucleotide library. In some instances, the amount of at least 2% of the polynucleotides in the library is 5, 10, 20, 25, 50, 75, 100, 150, 200, 250, 300, 400, 500, or 600 times higher than the mean representation for the polynucleotide library. In some instances, the amount of at least 5% of the polynucleotides in the library is 5, 10, 20, 25, 50, 75, 100, 150, 200, 250, 300, 400, 500, or 600 times higher than the mean representation for the polynucleotide library. In some instances, the amount of no more than 5% of the polynucleotides in the library is 5, 10, 20, 25, 50, 75, 100, 150, 200, 250, 300, 400, 500, or 600 times higher than the mean representation for the polynucleotide library. In some instances, the amount of no more than 10% of the polynucleotides in the library is 5, 10, 20, 25, 50, 75, 100, 150, 200, 250, 300, 400, 500, or 600 times higher than the mean representation for the polynucleotide library. In some instances, the amount of at least 1%-10% of the polynucleotides in the library is 5, 10, 20, 25, 50, 75, 100, 150, 200, 250, 300, 400, 500, or 600 times higher than the mean representation for the polynucleotide library. In some instances, the amount of at least 1%-20% of the polynucleotides in the library is 5, 10, 20, 25, 50, 75, 100, 150, 200, 250, 300, 400, 500, or 600 times higher than the mean representation for the polynucleotide library. In some instances, the relative amount of a polynucleotide library is adjusted based on high or low GC content.

Polynucleotide libraries for identifying variant sequences may collectively target a desired number of bases (bait territory). In some instances, a polynucleotide library comprise a bait territory of at least 5, 10, 15, 20, 25, 30, 40, 50, 60, 70, 80, 90 or at least 100 million bases. In some instances, a polynucleotide library comprise a bait territory of about 5, 10, 15, 20, 25, 30, 40, 50, 60, 70, 80, 90 or about 100 million bases. In some instances, a polynucleotide library comprise a bait territory of no more than 5, 10, 15, 20, 25, 30, 40, 50, 60, 70, 80, 90 or no more than 100 million bases.

### Unique Molecular Identifiers

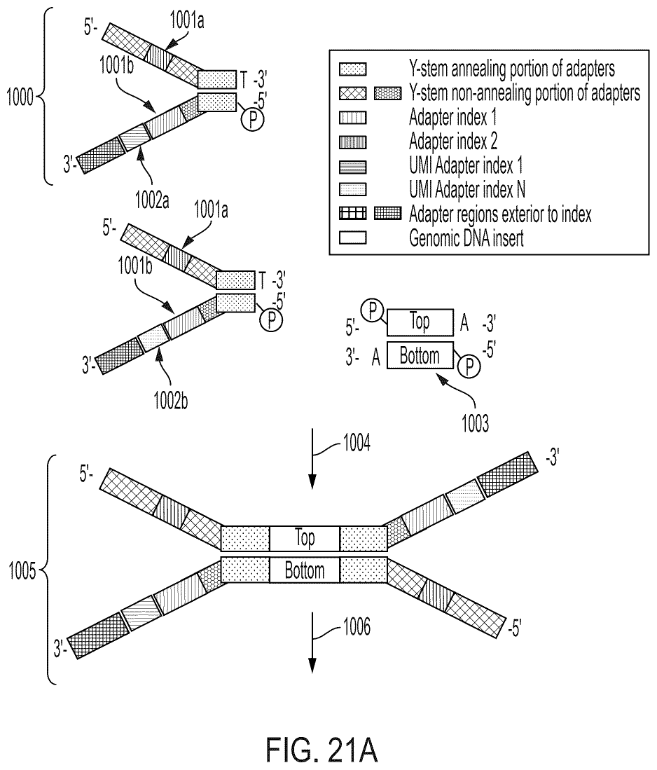

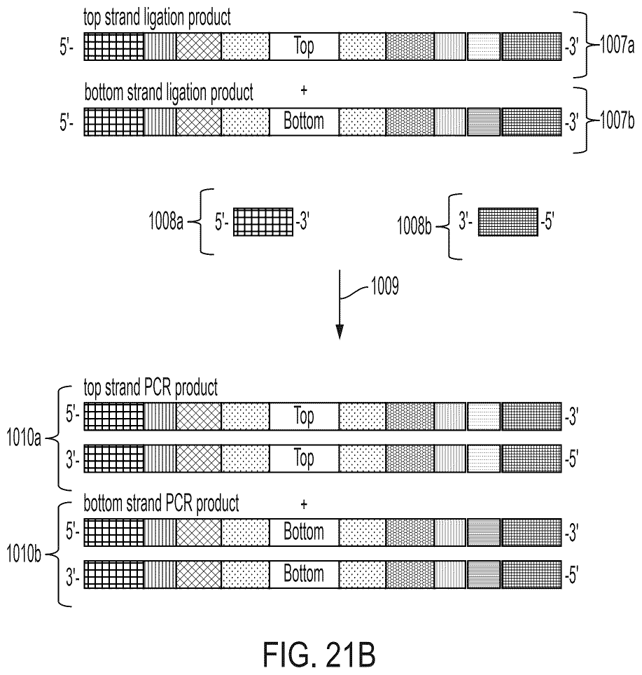

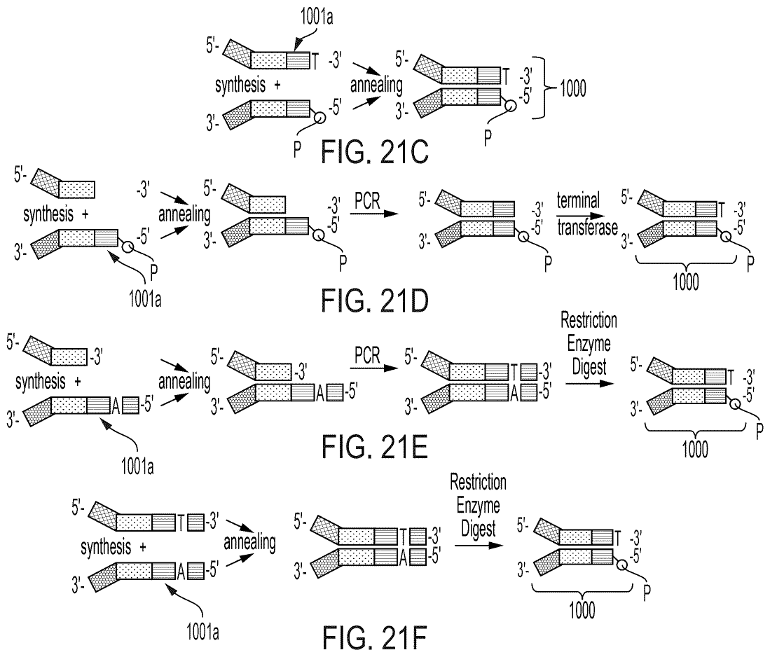

Described herein are adapters comprising unique molecular identifiers (UMIs). Adapters in some instances comprise a structure 1000 of FIG. 21. In some instances, adapters comprise universal adapters. In some instances adapters comprise a Y-annealing region (anneals to form yoke), one or more Y-step non-annealing regions, a first index region 1001a, a second index region 1001b, a first UMI (index) region 1002a, a second UMI (index) region 1002b, and one or more regions exterior to the index. In some instances, adapters 1000 are ligated 1004 to sample polynucleotides 1003 to form an adapter-ligated polynucleotide 1005. After denaturation 1006 of 1005 (FIG. 21A), top 1007a and bottom 1007b strand ligation products are formed. In some instances, each strand is labeled with a different UMI. After amplification 1009 with forward 1008a and backward 1008b primers, top strand 1010a and bottom strand 1010b PCR products are generated. In some instances, adapter ligated polynucleotides generated with universal adapters are further amplified with barcoded primers. In some instances adapters described herein comprise “in-line” UMIs, wherein at least one of a 5′ or 3′ UMI is not complementary to the other corresponding strand of the adapter (1001a and 1001b are not complementary). In some instances adapters described herein comprise “duplex” UMIs, wherein at least one of a 5′ or 3′ UMI is complementary to the other corresponding strand of the adapter (1001a and 1001b are complementary).

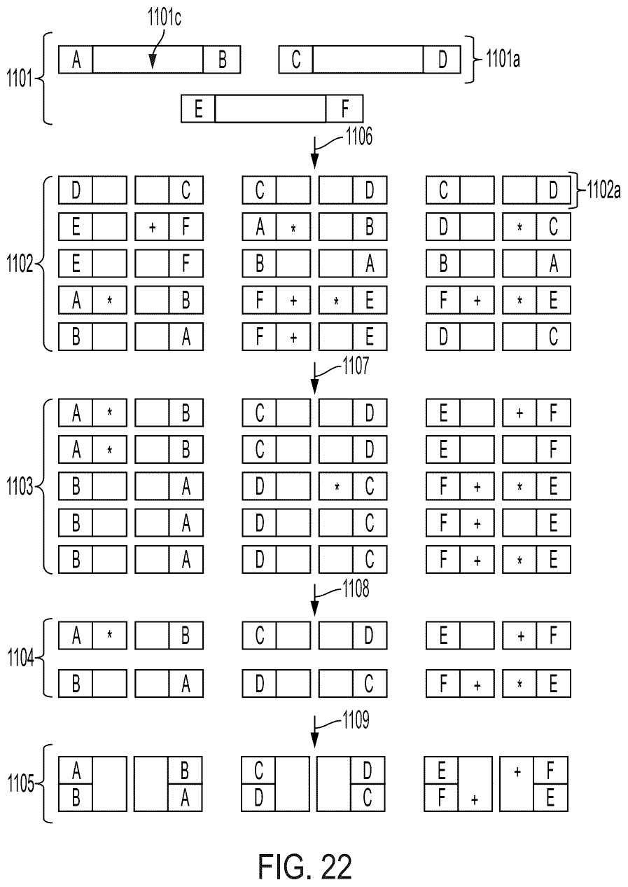

Adapter-ligated libraries comprising unique molecular identifiers may be used to distinguish between “true” mutations from a polynucleotide sample library and artifacts generated during sequencing library preparation (e.g., PCR errors, sequencing errors, or other erroneous base call). In some instances, a workflow as shown in FIG. 22 is used to analyze a library of adapter-ligated sample polynucleotides 1101. Adapter-ligated sample polynucleotides 1101 each comprise two distinct UMIs 1101b represented by letters (A-F; six combinations of barcodes are shown for simplicity), and are attached to a sample polynucleotide 1101c. After sequencing 1106, forward and reverse read pairs 1102 from sequencing are sorted into read pair groups 1102a. Potential PCR-based errors are designated with “*”, and true polymorphisms are designated as “+”. Next, read pairs 1103 are grouped 1107 by barcode and barcode position. Single-stranded consensus sequences 1104 are then generated 1108 from each group of barcode-grouped read pairs. Errors from D-C, and F-E are identified, although the error in A-B remains. Finally, duplex consensus sequences 1105 are generated 1109 by comparing each set of single stranded consensus sequences. The error in A-B can be identified, and true mutation E-F can be confirmed. In some instances, errors include substitutions, deletions, or insertions. In some instances, an error is present in the sample polynucleotide portion of an adapter-ligated polynucleotide. In some instances, an error is present in a barcode configured to identify a sample origin (e.g., index) or to uniquely identify a sample polynucleotide. In some instances, an error is present in a UMI. In some instances, an error is present in a sample index. Compositions and methods described herein in some instances are used to identify such errors.

Described herein are sets of UMIs, wherein the set has defined properties. In some instances, a UMI set comprises a plurality of different polynucleotides having unique sequences. In some instances, a UMI set is 8, 12, 16, 20, 24, 30, 32, 36, 39, 48, or 64 unique sequences. In some instances, the sequences of a UMI set differ by a Hamming distance of no more than 1, 2, 3, 4, or 5. In some instances, the sequences of a UMI set differ by a Hamming distance of at least 1, 2, 3, 4, or 5. In some instances, the sequences of a UMI set differ by a Hamming distance of at least 2. In some instances, the sequences of a UMI set differ by a Hamming distance of at least 1.

UMIs may be any length, depending on the desired application. In some instances, a UMI is no more than 15, 12, 10, 8, 7, 6, 5, 4, or not more than 3 bases in length. In some instances, a UMI is about 15, 12, 10, 8, 7, 6, 5, 4, or about 3 bases in length. In some instances, a UMI is about 3-12, 3-10, 3-8, 4-12, 4-10, 4-8, 6-12, or 8-12 bases in length. UMIs in a set may comprise more than one length. In some instances, 10, 20, 25, 30, 40, 50, 60, or 70 percent of UMIs in the set are a first length, and 90, 80, 75, 70, 60, 50, 40, or 30 percent are a second length. In some instances, the first length is 3-5 bases, and the second length is 3-5 bases. In some instances, UMIs comprise lengths of 5 or 6 bases.

After addition of UMI-containing adapters to sample polynucleotides, at least some of the sample polynucleotides may be uniquely labeled. In some instances, at least 30%, 50%, 75%, 80%, 90%, 95%, or at least 98% of the sample polynucleotides are ligated to adapters comprising UMIs. In some instances, at least 1%, 2%, 5%, 10%, 15%, 20%, 30%, 50%, 75%, 80%, 90%, 95%, or at least 98% of the sample polynucleotides are labeled with a unique UMI sequence. In some instances, no more than 1%, 2%, 5%, 10%, 15%, 20%, 30%, 50%, 75%, 80%, 90%, 95%, or no more than 98% of the sample polynucleotides are labeled with a unique UMI sequence. In some instances, at least 1%, 2%, 5%, 10%, 15%, 20%, 30%, 50%, 75%, 80%, 90%, 95%, or at least 98% of the sample polynucleotides are uniquely identifiable after labeling with a UMI.

UMIs described herein in some instances comprise sequences of one or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACAC, AATGC, ACTAG, AGCAT, AGTAC, ATCTC, CAGAC, CAGTA, CGAAT, CGGTT, CTTGG, GCATA, GCTAA, GTGAG, GTGTC, and TGTGC. UMIs described herein in some instances comprise sequences of two or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACAC, AATGC, ACTAG, AGCAT, AGTAC, ATCTC, CAGAC, CAGTA, CGAAT, CGGTT, CTTGG, GCATA, GCTAA, GTGAG, GTGTC, and TGTGC. UMIs described herein in some instances comprise sequences of five or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACAC, AATGC, ACTAG, AGCAT, AGTAC, ATCTC, CAGAC, CAGTA, CGAAT, CGGTT, CTTGG, GCATA, GCTAA, GTGAG, GTGTC, and TGTGC. UMIs described herein in some instances comprise sequences of ten or more of AAGGA, ACAAC, ATACG, CACTG, CATGA, CGATA, CGTGT, GCCAT, GCTGT, GTCAC, GTCGT, TACGA, TCCTA, TCGTG, TGTCG, TTGGC, AACAC, AATGC, ACTAG, AGCAT, AGTAC,

ATCTC, CAGAC, CAGTA, CGAAT, CGGTT, CTTGG, GCATA, GCTAA, GTGAG, GTGTC, and TGTGC.

UMIs may be represented at pre-selected percentages among a library of UMIs. In some instances at least 90% of the UMIs are present at fraction of 1-5%. In some instances at least 90% of the UMIs are present at fraction of 0.5%, 1%, 1.5%, 2%, 2.5%, 3%, 3.5%, 4%, 4.5%, 5%, 5.5%, 6%, 7%, or 8%. In some instances at least 90% of the UMIs are present at fraction of 0.5-8%, 1-7%, 1.5-7%, 2-7%, 2.5-6%, 3-8%, 3-6%, 1-5%, 0.5-5.5%, 1-4%, 1-6%, or 1-8%.

Any amount of sample polynucleotides (e.g., input DNA or other nucleic acid) may be ligated to adapters described herein. In some instances, the amount of sample polynucleotides is about 1, 5, 8, 10, 15, 20, 25, 30, 50, 75, or about 100 ng. In some instances, the amount of sample polynucleotides is no more than 1, 5, 8, 10, 15, 20, 25, 30, 50, 75, or no more than 100 ng. In some instances, the amount of sample polynucleotides is at least 1, 5, 8, 10, 15, 20, 25, 30, 50, 75, or at least 100 ng. In some instances, the amount of sample polynucleotides 1-10 ng, 1-100 ng, 3-10 ng, 5-100 ng, 5-75 ng, 5-50 ng, 10-100 ng, 10-50 ng, 25-100 ng, or 25-75 ng.

Provided herein are methods of generating adapters comprising UMIs. In a first method of adapter synthesis comprising synthesis of a top strand of an adapter comprising at least one UMI and a complementary bottom strand. After annealing the top and bottom adapter strands, an adapter comprising the structure of adapter 1000 is formed (FIG. 21C). In a second method of adapter synthesis, a top strand is synthesized without a UMI, and a bottom strand comprising a complementary region and a UMI (FIG. 21D). After, annealing, PCR is used to generate a complementary UMI on the top strand, and a terminal transferase adds a T to the 3′ end of top strand to generate adapter 1000. In a third method of synthesis, a top strand which does not comprise a UMI, and a bottom strand comprising a UMI, a restrictions site, and a 5′ overhang are synthesized (FIG. 21E). After annealing, the top strand is extended with PCR, and a restriction endonuclease is used to cleave a portion of the 3′ top strand and 5′ bottom strand to generate adapter 1000. In a fourth method of adapter synthesis, two complementary strands each comprising a UMI, a restriction site, and an overhang portion (3′ top strand, 5′ bottom strand) are synthesized, annealed, and cleaved with a restriction enzyme to generate adapter 1000. More than one UMIs may be present per adapter. In some instances, an adapter comprises 1, 2, 3, 4, 5, or more UMIs. In some instances, adapters comprise a first UMI and a second UMI. In some instances, a first UMI and a second UMI are complementary. In some instances, adapters comprise a first UMI and a second UMI. In some instances, a first UMI and a second UMI are not complementary. In some instances adapters are combined into libraries of adapters. In some instances adapters in a library comprise UMIs. In some instances adapters in a library comprise unique combinations of a first UMI and a second UMI.

### Universal Adapters

Provided herein are universal adapters. In some instances, universal adapters comprise one or more unique molecular identifiers. In some instances, the universal adapters disclosed herein may comprise a universal polynucleotide adapter comprising a first strand and a second strand. In some instances, a first strand comprises a first primer binding region, a first non-complementary region, and a first yoke region. In some instances, a second strand comprises a second primer binding region, a second non-complementary region, and a second yoke region. In some instances, a primer binding region allows for PCR amplification of a polynucleotide adapter. In some instances, a primer binding region allows for PCR amplification of a polynucleotide adapter and concurrent addition of one or more barcodes to the polynucleotide adapter. In some instances, the first yoke region is complementary to the second yoke region. In some instances, the first non-complementary region is not complementary to the second non-complementary region. In some instances, the universal adapter is a Y-shaped or forked adapter. In some instances, one or more yoke regions comprise nucleobase analogues that raise the Tm between a first yoke region and a second yoke region. Primer binding regions as described herein may be in the form of a terminal adapter region of a polynucleotide. In some instances, a universal adapter comprises one index sequence. In some instances, a universal adapter comprises one unique molecular identifier. In some instances, universal adapters are configured for use with barcoded primers, wherein after ligation, barcoded primers are added via PCR.

A universal (polynucleotide) adapter may be shortened relative to a typical barcoded adapter (e.g., full-length “Y adapter”). For example, a universal adapter strand is 20-45 bases in length. In some instances, a universal adapter strand is 25-40 bases in length. In some instances, a universal adapter strand is 30-35 bases in length. In some instances, a universal adapter strand is no more than 50 bases in length, no more than 45 bases in length, no more than 40 bases in length, no more than 35 bases in length, no more than 30 bases in length, or no more than 25 bases in length. In some instances, a universal adapter strand is about 25, 27, 30, 32, 34, 36, 38, 40, 42, 44, 46, 48, 50, 52, 54, 56, 58, or about 60 bases in length. In some instances, a universal adapter strand is about 60 base pairs in length. In some instances, a universal adapter strand is about 58 base pairs in length. In some instances, a universal adapter strand is about 52 base pairs in length. In some instances, a universal adapter strand is about 33 base pairs in length.

A universal adapter may be modified to facilitate ligation with a sample polynucleotide. For example, the 5′ terminus is phosphorylated. In some instances, a universal adapter comprises one or more non-native nucleobase linkages such as a phosphorothioate linkage. For example, a universal adapter comprises a phosphorothioate between the 3′ terminal base, and the base adjacent to the 3′ terminal base. A sample polynucleotide in some instances comprises nucleic acid from a variety of sources, such as DNA or RNA of human, bacterial, plant, animal, fungal, or viral origin. An adapter-ligated sample polynucleotide in some instances comprises a sample polynucleotide (e.g., sample nucleic acid) with adapters universal adapters ligated to both the 5′ and 3′ end of the sample polynucleotide to form an adapter-ligated polynucleotide. A duplex sample polynucleotide comprises both a first strand (forward) and a second strand (reverse).

Universal adapters may contain any number of different nucleobases (DNA, RNA, etc.), nucleobase analogues, or non-nucleobase linkers or spacers. For example, an adapter comprises one or more nucleobase analogues or other groups that enhance hybridization (Tₘ) between two strands of the adapter. In some instances, nucleobase analogues are present in the yoke region of an adapter. Nucleobase analogues and other groups include but are not limited to locked nucleic acids (LNAs), bicyclic nucleic acids (BNAs), C5-modified pyrimidine bases, 2′-O-methyl substituted RNA, peptide nucleic acids (PNAs), glycol nucleic acid (GNAs), threose nucleic acid (TNAs), xenonucleic acids (XNAs) morpholino backbone-modified bases, minor grove binders (MGBs), spermine, G-clamps, or a anthraquinone (Uaq) caps.

Universal adapters may comprise any number of nucleobase analogues (such as LNAs or BNAs), depending on the desired hybridization Tₘ. For example, an adapter comprises 1 to 20 nucleobase analogues. In some instances, an adapter comprises 1 to 8 nucleobase analogues. In some instances, an adapter comprises at least 1, 2, 3, 4, 5, 6, 7, 8, 9, 10, 11, 12, or at least 12 nucleobase analogues. In some instances, an adapter comprises about 1, 2, 3, 4, 5, 6, 7, 8, 9, 10, 11, 12, 13, 14, 15, or about 16 nucleobase analogues. In some instances, the number of nucleobase analogous is expressed as a percent of the total bases in the adapter. For example, an adapter comprises at least 1%, 2%, 5%, 10%, 12%, 18%, 24%, 30%, or more than 30% nucleobase analogues. In some instances, adapters (e.g., universal adapters) described herein comprise methylated nucleobases, such as methylated cytosine.

### Barcodes

Polynucleotide primers may comprise defined sequences, such as barcodes (or indices). Adapters in some instances comprise one or more barcodes. In some instances, an adapter comprises at least one indexing barcode and at least one unique molecular identifier barcode. Barcodes can be attached to universal adapters, for example, using PCR and barcoded primers to generate barcoded adapter-ligated sample polynucleotides. Primer binding sites, such as universal primer binding sites, facilitate simultaneous amplification of all members of a barcode primer library, or a subpopulation of members. In some instances, a primer binding site comprises a region that binds to a flow cell or other solid support during next generation sequencing. In some instances, a barcoded primer comprises a P5 sequence having the nucleic acid sequence 5′-AATGATACGGCGACCACCGA-3′ (SEQ ID NO: 52) or P7 sequence having nucleic acid sequence 5′-CAAGCAGAAGACGGCATACGAGAT-3′ (SEQ ID NO: 53). In some instances, primer binding sites are configured to bind to universal adapter sequences, and facilitate amplification and generation of barcoded adapters. In some instances, barcoded primers are no more than 60 bases in length. In some instances, barcoded primers are no more than 55 bases in length. In some instances, barcoded primers are 50-60 bases in length. In some instances, barcoded primers are about 60 bases in length. In some instances, barcodes described herein comprise methylated nucleobases, such as methylated cytosine.

The number of unique barcodes available for a barcode set (collection of unique barcodes or barcode combinations configured to be used together to unique define samples) may depend on the barcode length. In some instances, a Hamming distance is defined by the number of base differences between any two barcodes. In some instances, a Levenshtein distance is defined by the number changes needed to change one barcode into another (insertions, substitutions, or deletions). In some instances, barcode sets described herein comprise a Levenshtein distance of at least 2, 3, 4, 5, 6, 7, or at least 8. In some instances, barcode sets described herein comprise a Hamming distance of at least 2, 3, 4, 5, 6, 7, or at least 8.

Barcodes may be incorrectly associated with a different sample than they were assigned. In some instances, incorrect barcodes are occur from PCR errors (e.g., substitution) during library amplification. In some instances, entire barcodes “hop” or are transferred from one sample polynucleotide to another. Such transfers in some instances result from cross-contamination of free adapters or primers during a library generation workflow. In some instances a group of barcodes (barcode set) is chosen to minimize “barcode hopping”. In some instances, barcode hopping (for a single barcode) for a barcode set described herein is no more than 7%, 5%, 4%, 3%, 2%, 1%, 0.5%, or no more than 0.1%. In some instances, barcode hopping (for a single barcode) for a barcode set described herein is 0.1-6%, 0.1-5%, 0.2-5%, 0.5-5%, 1-7%, 1-5%, or 0.5-7%. In some instances, barcode hopping (for two barcodes) for a barcode set described herein is no more than 0.7%, 0.5%, 0.4%, 0.3%, 0.2%, 0.1%, 0.05%, or no more than 0.1%. In some instances, barcode hopping (for two barcodes) for a barcode set described herein is 0.01-0.6%, 0.01-0.5%, 0.02-0.5%, 0.05-0.5%, 0.1-0.7%, 0.1-0.5%, or 0.05-0.7%.

Barcoded primers comprise one or more barcodes. In some instances, the barcodes are added to universal adapters through PCR reaction. Barcodes are nucleic acid sequences that allow some feature of a polynucleotide with which the barcode is associated to be identified. In some instances, a barcode comprises an index sequence. In some instances, index sequences allow for identification of a sample, or unique source of nucleic acids to be sequenced. A barcode or combination of barcodes in some instances identifies a specific patient. A barcode or combination of barcodes in some instances identifies a specific sample from a patient among other samples from the same patient. After sequencing, the barcode (or barcode region) provides an indicator for identifying a characteristic associated with the coding region or sample source. Barcodes can be designed at suitable lengths to allow sufficient degree of identification, e.g., at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or more bases in length. Multiple barcodes, such as about 2, 3, 4, 5, 6, 7, 8, 9, 10, or more barcodes, may be used on the same molecule, optionally separated by non-barcode sequences. In some instances, a barcode is positioned on the 5′ and the 3′ sides of a sample polynucleotide. In some instances, each barcode in a plurality of barcodes differ from every other barcode in the plurality at least three base positions, such as at least about 3, 4, 5, 6, 7, 8, 9, 10, or more positions. Use of barcodes allows for the pooling and simultaneous processing of multiple libraries for downstream applications, such as sequencing (multiplex). In some instances, at least 4, 8, 16, 32, 48, 64, 128, or more 512 barcoded libraries are used. In some instances, at least 400, 500, 800, 1000, 2000, 5000, 10,000, 12,000, 15,000, 18,000, 20,000, or at 25,000 barcodes are used. Barcoded primers or adapters may comprise unique molecular identifiers (UMI). Such UMIs in some instances uniquely tag all nucleic acids in a sample. In some instances, at least 60%, 70%, 80%, 90%, 95%, or more than 95% of the nucleic acids in a sample are tagged with a UMI. In some instances, at least 85%, 90%, 95%, 97%, or at least 99% of the nucleic acids in a sample are tagged with a unique barcode, or UMI. Barcoded primers in some instances comprise an index sequence and one or more UMI. UMIs allow for internal measurement of initial sample concentrations or stoichiometry prior to downstream sample processing (e.g., PCR or enrichment steps) which can introduce bias. In some instances, UMIs comprise one or more barcode sequences. In some instances, each strand (forward vs. reverse) of an adapter-ligated sample polynucleotide possesses one or more unique barcodes. Such barcodes are optionally used to uniquely tag each strand of a sample polynucleotide. In some instances, a barcoded primer comprises an index barcode and a UMI barcode. In some instances, after amplification with at least two barcoded primers, the resulting amplicons comprise two index sequences and two UMIs. In some instances, after amplification with at least two barcoded primers, the resulting amplicons comprise two index barcodes and one UMI barcode. In some instances, each strand of a universal adapter-sample polynucleotide duplex is tagged with a unique barcode, such as a UMI or index barcode.

Barcoded primers in a library comprise a region that is complementary to a primer binding region on a universal adapter. For example, universal adapter binding region is complementary to primer region of the universal adapter, and universal adapter binding region is complementary to primer region of the universal adapter. Such arrangements facilitate extension of universal adapters during PCR, and attach barcoded primers. In some instances, the Tm between the primer and the primer binding region is 40-65 degrees C. In some instances, the Tm between the primer and the primer binding region is 42-63 degrees C. In some instances, the Tm between the primer and the primer binding region is 50-60 degrees C. In some instances, the Tm between the primer and the primer binding region is 53-62 degrees C. In some instances, the Tm between the primer and the primer binding region is 54-58 degrees C. In some instances, the Tm between the primer and the primer binding region is 40-57 degrees C. In some instances, the Tm between the primer and the primer binding region is 40-50 degrees C. In some instances, the Tm between the primer and the primer binding region is about 40, 45, 47, 50, 52, 53, 55, 57, 59, 61, or 62 degrees C.

### Hybridization Blockers

Blockers may contain any number of different nucleobases (DNA, RNA, etc.), nucleobase analogues (non-canonical), or non-nucleobase linkers or spacers. In some instances, blockers comprise universal blockers. Such blockers may in some instances are described as a “set”, wherein the set comprises two or more blockers configured to prevent unwanted interactions with the same adapter sequence. In some instances, universal blockers prevent adapter-adapter interactions independent of one or more barcodes present on at least one of the adapters. For example, a blocker comprises one or more nucleobase analogues or other groups that enhance hybridization (Tₘ) between the blocker and the adapter. In some instances, a blocker comprises one or more nucleobases which decrease hybridization (Tₘ) between the blocker and the adapter (e.g., “universal” bases). In some instances, a blocker described herein comprises both one or more nucleobases which increase hybridization (Tₘ) between the blocker and the adapter and one or more nucleobases which decrease hybridization (Tₘ) between the blocker and the adapter.

Described herein are hybridization blockers comprising one or more regions which enhance binding to targeted sequences (e.g., adapter), and one or more regions which decrease binding to target sequences (e.g., adapter). In some instances, each region is tuned for a given desired level of off-bait activity during target enrichment applications. In some instances, each region can be altered with either a single type of chemical modification/moiety or multiple types to increase or decrease overall affinity of a molecule for a targeted sequence. In some instances, the melting temperature of all individual members of a blocker set are held above a specified temperature (e.g., with the addition of moieties such as LNAs and/or BNAs). In some instances, a given set of blockers will improve off bait performance independent of index length, independent of index sequence, and independent of how many adapter indices are present in hybridization.

Blockers may comprise moieties which increase and/or decrease affinity for a target sequencing, such as an adapter. In some instances, such specific regions can be thermodynamically tuned to specific melting temperatures to either avoid or increase the affinity for a particular targeted sequence. This combination of modifications is in some instances designed to help increase the affinity of the blocker molecule for specific and unique adapter sequence and decrease the affinity of the blocker molecule for repeated adapter sequence (e.g., Y-stem annealing portion of adapter). In some instances, blockers comprise moieties which decrease binding of a blocker to the Y-stem region of an adapter. In some instances, blockers comprise moieties which decrease binding of a blocker to the Y-stem region of an adapter, and moieties which increase binding of a blocker to non-Y-stem regions of an adapter.

Blockers (e.g., universal blockers) and adapters may form a number of different populations during hybridization. In a population ‘A’ in some instances comprises blockers correctly bound to non-index regions of the adapters. In a population ‘B’, a region of the blockers is bound to the “yoke” region of the adapter, but a remaining portion of the blocker does not bind to an adjacent region of the adapter. In a population ‘C’, two blockers unproductively dimerize. In a population ‘D’, blockers are unbound to any other nucleic acids. In some instances, when the number of DNA modifications that decrease affinity in the Y-stem annealing region of the blocker are increased, the populations ‘A’ & ‘D’ dominate and either have the desired or minimal effect. In some instances, as the number of DNA modifications that decrease affinity in the Y-stem annealing region of the blocker are decreased, the populations ‘B’ & ‘C’ dominate and have undesired effects where daisy-chaining or annealing to other adapters can occur (‘B’) or sequester blockers where they are unable to function properly (‘C’).

The index on both single- and dual-index adapter designs may be either partially or fully covered by universal blockers that have been extended with specifically designed DNA modifications to cover adapter index bases. In some instances, such modifications comprise moieties which decrease annealing to the index, such as universal bases. In some instances, the index of a dual index adapter is partially covered (or is overlapped) by one or more blockers. In some instances, the index of a dual index adapter is fully covered by one or more blockers. In some instances, the index of a single index adapter is partially covered by one or more blockers. In some instances, the index of a single index adapter is fully covered by one or more blockers. In some instances, a blocker overlaps an index sequence by at least 1, 2, 3, 4, 5, 6, 7, 8, 9, 10, 11, 12, 13, 14, 15, 20 or more than 20 bases. In some instances, a blocker overlaps an index sequence by no more than 1, 2, 3, 4, 5, 6, 7, 8, 9, 10, 11, 12, 13, 14, 15, 20, or no more than 25 bases. In some instances, a blocker overlaps an index sequence by about 1, 2, 3, 4, 5, 6, 7, 8, 9, 10, 11, 12, 13, 14, 15, 20 or about 30 bases. In some instances, a blocker overlaps an index sequence by 1-5, 1-3, 2-5, 2-8, 2-10, 3-6, 3-10, 4-10, 4-15, 1-4 or 5-7 bases. In some instances, a region of a blocker which overlaps an index sequences comprises at least one 2-deoxyinosine or 5-nitroindole nucleobase.

One or two blockers may overlap with an index sequence present on an adapter. In some instances, one or two blockers combined overlap with at least 1, 2, 3, 4, 5, 6, 7, 8, 9, 10, 11, 12, 13, 14, 15, 20 or more than 20 bases of the index sequence. In some instances, one or two blockers combined overlap with no more than 1, 2, 3, 4, 5, 6, 7, 8, 9, 10, 11, 12, 13, 14, 15, 20 or no more than 20 bases of the index sequence. In some instances, one or two blockers combined overlap with about 1, 2, 3, 4, 5, 6, 7, 8, 9, 10, 11, 12, 13, 14, 15, 20 or about 20 bases of the index sequence. In some instances, one or two blockers combined overlap by 1-5, 1-3, 2-5, 2-8, 2-10, 3-6, 3-10, 4-10, 4-15, 1-4 or 5-7 bases of the index sequence. In some instances, a region of a blocker which overlaps an index sequences comprises at least one 2-deoxyinosine or 5-nitroindole nucleobase.

In a first arrangement, the length of the adapter index overhang may be varied. When designed from a single side, the adapter index overhang can be altered to cover from 0 to n of the adapter index bases from either side of the index. This allows for the ability to design such adapter blockers for both single and dual index adapter systems.

In a second arrangement, the adapter index bases are covered from both sides. When adapter index bases are covered from both sides, the length of the covering region of each blocker can be chosen such that a single pair of blockers is capable of interacting with a range of adapter index lengths while still covering a significant portion of the total number of index bases. As an example, take two blockers that have been designed with 3 bp overhangs that cover the adapter index. In the context of 6 bp, 8 bp, or 10 bp adapter index lengths, these blockers will leave 0 bp, 2 bp, or 4 bp exposed during hybridization, respectively.

In a third arrangement, modified nucleobases are selected to cover index adapter bases. Examples of these modifications that are currently commercially available include degenerate bases (i.e., mixed bases of A, T, C, G), 2′-deoxyInosine, & 5-nitroindole.

In a forth arrangement, blockers with adapter index overhangs bind to either the sense (i.e., “top”) or anti-sense (i.e., “bottom”) strand of a next generation sequencing library.

In a fifth arrangement, blockers are further extended to cover other polynucleotide sequences (e.g., a poly-A tail added in a previous biochemical step in order to facilitate ligation or other method to introduce a defined adapter sequence, unique molecular identifier for bioinformatic assignment following sequencing, etc.) in addition to the standard adapter index bases of defined length and composition. These types of sequences can be placed in multiple locations of an adapter and in this case the most widely utilized case (i.e., unique molecular index next to the genomic insert) is presented. Other positions for the unique molecular identifier (e.g., next to adapter index bases) could also be addressed with similar approaches.

In a sixth arrangement, all of the previous arrangements are utilized in various combinations to meet a targeted performance metric for off-bait performance during target enrichment under specified conditions.

Blockers may comprise moieties, such as nucleobase analogues. Nucleobase analogues and other groups include but are not limited to locked nucleic acids (LNAs), bicyclic nucleic acids (BNAs), C5-modified pyrimidine bases, 2′-O-methyl substituted RNA, peptide nucleic acids (PNAs), glycol nucleic acid (GNAs), threose nucleic acid (TNAs), inosine, 2′-deoxyInosine, 3-nitropyrrole, 5-nitroindole, xenonucleic acids (XNAs) morpholino backbone-modified bases, minor grove binders (MGBs), spermine, G-clamps, or a anthraquinone (Uaq) caps. In some instances, nucleobase analogues comprise universal bases, wherein the nucleobase has a lower Tₘ for binding to a cognate nucleobase. In some instances, universal bases comprise 5-nitroindole or 2′-deoxyInosine. In instances, blockers comprise spacer elements that connect two polynucleotide chains. In some instances, blockers comprise one or more nucleobase analogues. In some instances, such nucleobase analogues are added to control the Tₘ of a blocker. Blockers may comprise any number of nucleobase analogues (such as LNAs or BNAs), depending on the desired hybridization Tₘ. For example, a blocker comprises 20 to 40 nucleobase analogues. In some instances, a blocker comprises 8 to 16 nucleobase analogues. In some instances, a blocker comprises at least 1, 2, 3, 4, 5, 6, 7, 8, 9, 10, 11, 12, or at least 12 nucleobase analogues. In some instances, a blocker comprises about 1, 2, 3, 4, 5, 6, 7, 8, 9, 10, 11, 12, 13, 14, 15, or about 16 nucleobase analogues. In some instances, the number of nucleobase analogous is expressed as a percent of the total bases in the blocker. For example, a blocker comprises at least 1%, 2%, 5%, 10%, 12%, 18%, 24%, 30%, or more than 30% nucleobase analogues. In some instances, the blocker comprising a nucleobase analogue raises the Tₘ in a range of about 2° C. to about 8° C. for each nucleobase analogue. In some instances, the Tₘ is raised by at least or about 1° C., 2° C., 3° C., 4° C., 5° C., 6° C., 7° C., 8° C., 9° C., 10° C., 12° C., 14° C., or 16° C. for each nucleobase analogue. Such blockers in some instances are configured to bind to the top or “sense” strand of an adapter. Blockers in some instances are configured to bind to the bottom or “anti-sense” strand of an adapter. In some instances a set of blockers includes sequences which are configured to bind to both top and bottom strands of an adapter. Additional blockers in some instances are configured to the complement, reverse, forward, or reverse complement of an adapter sequence. In some instances, a set of blockers targeting a top (binding to the top) or bottom strand (or both) is designed and tested, followed by optimization, such as replacing a top blocker with a bottom blocker, or a bottom blocker with a top blocker. In some instances, a blocker is configured to overlap fully or partially with bases of an index or barcode on an adapter. A set of blockers in some instances comprise at least one blocker overlapping with an adapter index sequence. A set of blockers in some instances comprise at least one blocker overlapping with an adapter index sequence, and at least one blocker which does not overlap with an adapter sequence. A set of blockers in some instances comprise at least one blocker which does not overlap with a yoke region sequence. A set of blockers in some instances comprise at least one blocker which does not overlap with a yoke region sequence and at least one blocker which overlaps with a yoke region sequence. A sets of blockers in some instances comprises 2, 3, 4, 5, 6, 7, 8, 9, 10, or more than 10 blockers.

Blockers may be any length, depending on the size of the adapter or hybridization Tₘ. For example, blockers are 20 to 50 bases in length. In some instances, blockers are 25 to 45 bases, 30 to 40 bases, 20 to 40 bases, or 30 to 50 bases in length. In some instances, blockers are 25 to 35 bases in length. In some instances blockers are at least 25, 26, 27, 28, 29, 30, 31, 32, 33, 34, or at least 35 bases in length. In some instances, blockers are no more than 25, 26, 27, 28, 29, 30, 31, 32, 33, 34, or no more than 35 bases in length. In some instances, blockers are about 25, 26, 27, 28, 29, 30, 31, 32, 33, 34, or about 35 bases in length. In some instances, blockers are about 50 bases in length. A set of blockers targeting an adapter-tagged genomic library fragment in some instances comprises blockers of more than one length. Two blockers are in some instances tethered together with a linker. Various linkers are well known in the art, and in some instances comprise alkyl groups, polyether groups, amine groups, amide groups, or other chemical group. In some instances, linkers comprise individual linker units, which are connected together (or attached to blocker polynucleotides) through a backbone such as phosphate, thiophosphate, amide, or other backbone. In an exemplary arrangement, a linker spans the index region between a first blocker that each targets the 5′ end of the adapter sequence and a second blocker that targets the 3′ end of the adapter sequence. In some instances, capping groups are added to the 5′ or 3′ end of the blocker to prevent downstream amplification. Capping groups variously comprise polyethers, polyalcohols, alkanes, or other non-hybridizable group that prevents amplification. Such groups are in some instances connected through phosphate, thiophosphate, amide, or other backbone. In some instances, one or more blockers are used. In some instances, at least 4 non-identical blockers are used. In some instances, a first blocker spans a first 3′ end of an adaptor sequence, a second blocker spans a first 5′ end of an adaptor sequence, a third blocker spans a second 3′ end of an adaptor sequence, and a fourth blockers spans a second 5′ end of an adaptor sequence. In some instances a first blocker is at least 20, 21, 22, 23, 24, 25, 26, 27, 28, 29, 30, 31, 32, 33, 34, or at least 35 bases in length. In some instances a second blocker is at least 20, 21, 22, 23, 24, 25, 26, 27, 28, 29, 30, 31, 32, 33, 34, or at least 35 bases in length. In some instances a third blocker is at least 20, 21, 22, 23, 24, 25, 26, 27, 28, 29, 30, 31, 32, 33, 34, or at least 35 bases in length. In some instances a fourth blocker is at least 20, 21, 22, 23, 24, 25, 26, 27, 28, 29, 30, 31, 32, 33, 34, or at least 35 bases in length. In some instances, a first blocker, second blocker, third blocker, or fourth blocker comprises a nucleobase analogue. In some instances, the nucleobase analogue is LNA.

The design of blockers may be influenced by the desired hybridization Tₘ to the adapter sequence. In some instances, non-canonical nucleic acids (e.g., locked nucleic acids, bridged nucleic acids, or other non-canonical nucleic acid or analog) are inserted into blockers to increase or decrease the blocker's Tₘ. In some instances, the Tₘ of a blocker is calculated using a tool specific to calculating Tₘ for polynucleotides comprising a non-canonical amino acid. In some instances, a Tₘ is calculated using the Exiqon™ online prediction tool. In some instances, blocker Tₘ described herein are calculated in-silico. In some instances, the blocker Tₘ is calculated in-silico, and is correlated to experimental in-vitro conditions. Without being bound by theory, an experimentally determined Tₘ may be further influenced by experimental parameters such as salt concentration, temperature, presence of additives, or other factor. In some instances, Tₘ described herein are in-silico determined Tₘ that are used to design or optimize blocker performance. In some instances, Tₘ values are predicted, estimated, or determined from melting curve analysis experiments. In some instances, blockers have a Tₘ of 70 degrees C. to 99 degrees C. In some instances, blockers have a Tₘ of 75 degrees C. to 90 degrees C. In some instances, blockers have a Tₘ of at least 85 degrees C. In some instances, blockers have a Tₘ of at least 70, 72, 75, 77, 80, 82, 85, 88, 90, or at least 92 degrees C. In some instances, blockers have a Tₘ of about 70, 72, 75, 77, 80, 82, 85, 88, 90, 92, or about 95 degrees C. In some instances, blockers have a Tₘ of 78 degrees C. to 90 degrees C. In some instances, blockers have a Tₘ of 79 degrees C. to 90 degrees C. In some instances, blockers have a Tₘ of 80 degrees C. to 90 degrees C. In some instances, blockers have a Tₘ of 81 degrees C. to 90 degrees C. In some instances, blockers have a Tₘ of 82 degrees C. to 90 degrees C. In some instances, blockers have a Tₘ of 83 degrees C. to 90 degrees C. In some instances, blockers have a Tₘ of 84 degrees C. to 90 degrees C. In some instances, a set of blockers have an average Tₘ of 78 degrees C. to 90 degrees C. In some instances, a set of blockers have an average Tₘ of 80 degrees C. to 90 degrees C. In some instances, a set of blockers have an average Tₘ of at least 80 degrees C. In some instances, a set of blockers have an average Tₘ of at least 81 degrees C. In some instances, a set of blockers have an average Tₘ of at least 82 degrees C. In some instances, a set of blockers have an average Tₘ of at least 83 degrees C. In some instances, a set of blockers have an average Tₘ of at least 84 degrees C. In some instances, a set of blockers have an average Tₘ of at least 86 degrees C. Blocker Tₘ are in some instances modified as a result of other components described herein, such as use of a fast hybridization buffer and/or hybridization enhancer.

The molar ratio of blockers to adapter targets may influence the off-bait (and subsequently off-target) rates during hybridization. The more efficient a blocker is at binding to the target adapter, the less blocker is required. Blockers described herein in some instances achieve sequencing outcomes of no more than 20% off-target reads with a molar ratio of less than 20:1 (blocker: target). In some instances, no more than 20% off-target reads are achieved with a molar ratio of less than 10:1 (blocker: target). In some instances, no more than 20% off-target reads are achieved with a molar ratio of less than 5:1 (blocker: target). In some instances, no more than 20% off-target reads are achieved with a molar ratio of less than 2:1 (blocker: target). In some instances, no more than 20% off-target reads are achieved with a molar ratio of less than 1.5:1 (blocker: target). In some instances, no more than 20% off-target reads are achieved with a molar ratio of less than 1.2:1 (blocker: target). In some instances, no more than 20% off-target reads are achieved with a molar ratio of less than 1.05:1 (blocker: target).

The universal blockers may be used with panel libraries of varying size. In some embodiments, the panel libraries comprises at least or about 0.01, 0.02, 0.03, 0.04, 0.05, 0.06, 0.07, 0.08, 0.09, 1.0, 2.0, 4.0, 8.0, 10.0, 12.0, 14.0, 16.0, 18.0, 20.0, 22.0, 24.0, 26.0, 28.0, 30.0, 40.0, 50.0, 60.0, or more than 60.0 megabases (Mb).

Blockers as described herein may improve on-target performance. In some embodiments, on-target performance is improved by at least or about 5%, 10%, 15%, 20%, 25%, 30%, 35%, 40%, 45%, 50%, 55%, 60%, 65%, 70%, 75%, 80%, 85%, 90%, 95%, or more than 95%. In some embodiments, the on-target performance is improved by at least or about 5%, 10%, 15%, 20%, 25%, 30%, 35%, 40%, 45%, 50%, 55%, 60%, 65%, 70%, 75%, 80%, 85%, 90%, 95%, or more than 95% for various index designs. In some embodiments, the on-target performance is improved by at least or about 5%, 10%, 15%, 20%, 25%, 30%, 35%, 40%, 45%, 50%, 55%, 60%, 65%, 70%, 75%, 80%, 85%, 90%, 95%, or more than 95% is improved for various panel sizes.

### De Novo Synthesis of Small Polynucleotide Populations for Amplification Reactions

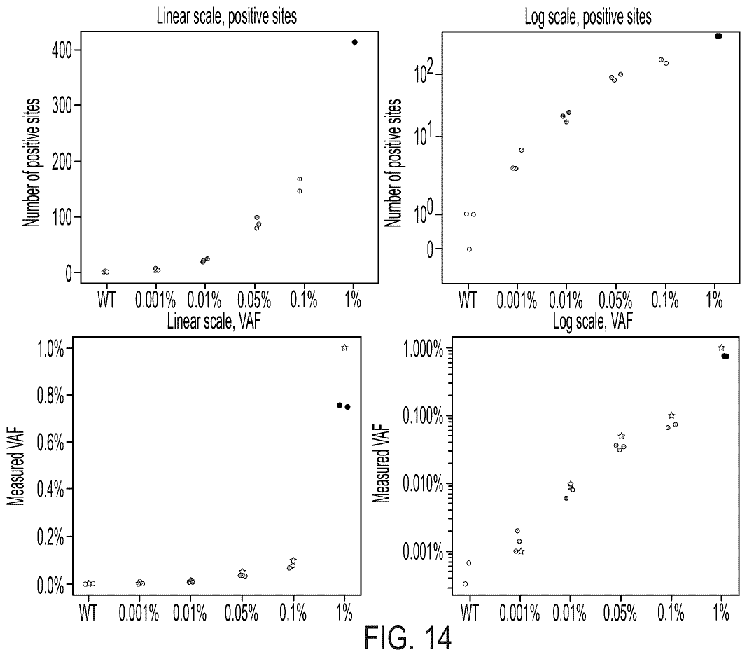

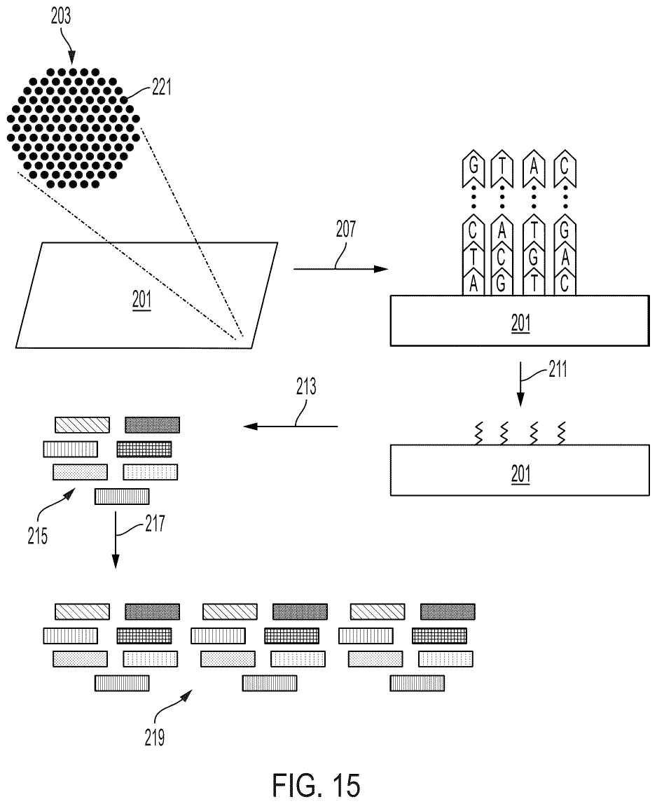

Described herein are methods of synthesis of polynucleotides from a surface, e.g., a plate (FIG. 15). In some instances, polynucleotide libraries comprise sample polynucleotide libraries. In some instances, the polynucleotides are synthesized on a cluster of loci for polynucleotide extension, released and then subsequently subjected to an amplification reaction, e.g., PCR. An exemplary workflow of synthesis of polynucleotides from a cluster is depicted in FIG. 15. A silicon plate 201 includes multiple clusters 203. Within each cluster are multiple loci 221. Polynucleotides are synthesized 207 de novo on a plate 201 from the cluster 203. Polynucleotides are cleaved 211 and removed 213 from the plate to form a population of released polynucleotides 215. The population of released polynucleotides 215 is then amplified 217 to form a library of amplified polynucleotides 219.

Provided herein are methods where amplification of polynucleotides synthesized on a cluster provide for enhanced control over polynucleotide representation compared to amplification of polynucleotides across an entire surface of a structure without such a clustered arrangement. In some instances, amplification of polynucleotides synthesized from a surface having a clustered arrangement of loci for polynucleotides extension provides for overcoming the negative effects on representation due to repeated synthesis of large polynucleotide populations. Exemplary negative effects on representation due to repeated synthesis of large polynucleotide populations include, without limitation, amplification bias resulting from high/low GC content, repeating sequences, trailing adenines, secondary structure, affinity for target sequence binding, or modified nucleotides in the polynucleotide sequence.

Cluster amplification as opposed to amplification of polynucleotides across an entire plate without a clustered arrangement can result in a tighter distribution around the mean. For example, if 100,000 reads are randomly sampled, an average of 8 reads per sequence would yield a library with a distribution of about 1.5× from the mean. In some cases, single cluster amplification results in at most about 1.5×, 1.6×, 1.7×, 1.8×, 1.9×, or 2.0× from the mean. In some cases, single cluster amplification results in at least about 1.0×, 1.2×, 1.3×, 1.5×1.6×, 1.7×, 1.8×, 1.9×, or 2.0× from the mean.

Cluster amplification methods described herein when compared to amplification across a plate can result in a polynucleotide library that requires less sequencing for equivalent sequence representation. In some instances at least 10%, at least 20%, at least 30%, at least 40%, at least 50%, at least 60%, at least 70%, at least 80%, at least 90%, or at least 95% less sequencing is required. In some instances up to 10%, up to 20%, up to 30%, up to 40%, up to 50%, up to 60%, up to 70%, up to 80%, up to 90%, or up to 95% less sequencing is required. Sometimes 30% less sequencing is required following cluster amplification compared to amplification across a plate. Sequencing of polynucleotides in some instances is verified by high-throughput sequencing such as by next generation sequencing. Sequencing of the sequencing library can be performed with any appropriate sequencing technology, including but not limited to single-molecule real-time (SMRT) sequencing, polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. The number of times a single nucleotide or polynucleotide is identified or “read” is defined as the sequencing depth or read depth. In some cases, the read depth is referred to as a fold coverage, for example, 55 fold (or 55×) coverage, optionally describing a percentage of bases.

In some instances, amplification from a clustered arrangement compared to amplification across a plate results in less dropouts, or sequences which are not detected after sequencing of amplification product. Dropouts can be of AT and/or GC. In some instances, a number of dropouts are at most about 1%, 2%, 3%, 4%, or 5% of a polynucleotide population. In some cases, the number of dropouts is zero.

A cluster as described herein comprises a collection of discrete, non-overlapping loci for polynucleotide synthesis. A cluster can comprise about 50-1000, 75-900, 100-800, 125-700, 150-600, 200-500, or 300-400 loci. In some instances, each cluster includes 121 loci. In some instances, each cluster includes about 50-500, 50-200, 100-150 loci. In some instances, each cluster includes at least about 50, 100, 150, 200, 500, 1000 or more loci. In some instances, a single plate includes 100, 500, 10000, 20000, 30000, 50000, 100000, 500000, 700000, 1000000 or more loci. A locus can be a spot, well, microwell, channel, or post. In some instances, each cluster has at least 1×, 2×, 3×, 4×, 5×, 6×, 7×, 8×, 9×, 10×, or more redundancy of separate features supporting extension of polynucleotides having identical sequence.

Generation of Polynucleotide Libraries with Controlled Stoichiometry of Sequence Content

In some instances, the polynucleotide library (such as a sample polynucleotide set for variant detection) is synthesized with a specified distribution of desired polynucleotide sequences. In some instances, adjusting polynucleotide libraries for enrichment of specific desired sequences results in improved downstream application outcomes.

One or more specific sequences can be selected based on their evaluation in a downstream application. In some instances, the evaluation is binding affinity to target sequences for amplification, enrichment, or detection, stability, melting temperature, biological activity, ability to assemble into larger fragments, or other property of polynucleotides. In some instances, the evaluation is empirical or predicted from prior experiments and/or computer algorithms. An exemplary application includes increasing sequences in a probe library which correspond to areas of a genomic target having less than average read depth.

Selected sequences in a polynucleotide library can be at least 10%, 20%, 30%, 40%, 50%, 60%, 70%, 80%, 90%, 95%, or more than 95% of the sequences. In some instances, selected sequences in a polynucleotide library are at most 10%, 20%, 30%, 40%, 50%, 60%, 70%, 80%, 90%, 95%, or at most 100% of the sequences. In some cases, selected sequences are in a range of about 5-95%, 10-90%, 30-80%, 40-75%, or 50-70% of the sequences.

Polynucleotide libraries can be adjusted for the frequency of each selected sequence. In some instances, polynucleotide libraries favor a higher number of selected sequences. For example, a library is designed where increased polynucleotide frequency of selected sequences is in a range of about 40% to about 90%. In some instances, polynucleotide libraries contain a low number of selected sequences. For example, a library is designed where increased polynucleotide frequency of the selected sequences is in a range of about 10% to about 60%. A library can be designed to favor a higher and lower frequency of selected sequences. In some instances, a library favors uniform sequence representation. For example, polynucleotide frequency is uniform with regard to selected sequence frequency, in a range of about 10% to about 90%. In some instances, a library comprises polynucleotides with a selected sequence frequency of about 10% to about 95% of the sequences.

Generation of polynucleotide libraries with a specified selected sequence frequency in some cases occurs by combining at least 2 polynucleotide libraries with different selected sequence frequency content. In some instances, at least 2, 3, 4, 5, 6, 7, 10, or more than 10 polynucleotide libraries are combined to generate a population of polynucleotides with a specified selected sequence frequency. In some cases, no more than 2, 3, 4, 5, 6, 7, or 10 polynucleotide libraries are combined to generate a population of non-identical polynucleotides with a specified selected sequence frequency.

In some instances, selected sequence frequency is adjusted by synthesizing fewer or more polynucleotides per cluster. For example, at least 25, 50, 100, 200, 300, 400, 500, 600, 700, 800, 900, 1000, or more than 1000 non-identical polynucleotides are synthesized on a single cluster. In some cases, no more than about 50, 100, 200, 300, 400, 500, 600, 700, 800, 900, 1000 non-identical polynucleotides are synthesized on a single cluster. In some instances, 50 to 500 non-identical polynucleotides are synthesized on a single cluster. In some instances, 100 to 200 non-identical polynucleotides are synthesized on a single cluster. In some instances, about 100, about 120, about 125, about 130, about 150, about 175, or about 200 non-identical polynucleotides are synthesized on a single cluster.

In some cases, selected sequence frequency is adjusted by synthesizing non-identical polynucleotides of varying length. For example, the length of each of the non-identical polynucleotides synthesized may be at least or about at least 10, 15, 20, 25, 30, 35, 40, 45, 50, 100, 150, 200, 300, 400, 500, 2000 nucleotides, or more. The length of the non-identical polynucleotides synthesized may be at most or about at most 2000, 500, 400, 300, 200, 150, 100, 50, 45, 35, 30, 25, 20, 19, 18, 17, 16, 15, 14, 13, 12, 11, 10 nucleotides, or less. The length of each of the non-identical polynucleotides synthesized may fall from 10-2000, 10-500, 9-400, 11-300, 12-200, 13-150, 14-100, 15-50, 16-45, 17-40, 18-35, and 19-25.

### Use of Polynucleotide Libraries as Standards or Detection

Provided herein are methods of using polynucleotide libraries to improve the sensitivity and accuracy of nucleic acid variant detection. In some instances, the method comprises preparing a nucleic acid sample useful for determining the detection limit of genomic variants. In some instances, the method comprises one or more of the steps of providing a polynucleotide library described herein (e.g., reference standard); obtaining at least one sample from a patient suspected of having a disease or condition; detecting the presence or absence of the one or more variant sequences in the library; and detecting the presence or absence of the one or more variant sequences in the at least one sample. In some instances, detecting comprises sequencing. In some instances, detecting comprises Next Generation Sequencing (NGS). In some instances, sequencing comprises sequencing by synthesis, nanopore sequencing, SMRT sequencing, or other sequencing method described herein. In some instances, detecting comprises ddPCR or specific hybridization to an array.

Also provided herein are methods for using the polynucleotide libraries to detect variant sequences in a sample. The polynucleotides in the libraries themselves can comprise the variant. The polynucleotide libraries may be used to detect variant sequences having low variant allele frequencies. The at least one variant is present at a frequency of about 0.001% to 0.1% in the sample. In some instances, the polynucleotides libraries may be used to detect MRD. In some examples, the method comprises providing the at least one variant sequence. The variant sequence may be associated with MRD. In some instances, the polynucleotide libraries are used to detect minimal residual disease (MRD) in a sample. In some instances, the method comprises providing a library provided herein. The polynucleotides in the library can comprise the at least one variant for detection (e.g., variant associated with MRD). In some instances, the method comprises contacting the library with a sample. In some instances, the method comprises detecting a presence or an absence of the one or more variant sequences associated with a disease or condition, such as MRD, in the sample. In some instances, a recall of the one or more variant sequences is at least 5% greater than a plurality of polynucleotides without the one or more variant sequences. In some instances, a recall of the one or more variant sequences is 5% to 10% greater than a plurality of polynucleotides without the one or more variant sequences.

In some instances, the method further comprises ligating sequencing adapters to at least some polynucleotides in the test sample, the library, or both. In some instances, the method further comprises amplifying at least some polynucleotides in the sample, the library, or both.

The method can comprise obtaining the sample from an individual. In some instances, the individual was previously treated, is currently treated, or has received a clinical diagnosis for cancer. In some instances, the sample comprises a liquid biopsy. In some instances, the sample comprises circulating tumor DNA (ctDNA). In some instances, the sample is obtained from blood. In some examples, the sample is a biological sample obtained from the kidney, lung, breast, CRC, or melanoma. In some instances, the sample is substantially cell-free. In some instances, the polynucleotides comprise variant sequences corresponding to somatic variants found in diseases such as cancers, for example somatic variants found in breast, lung, CRC, melanoma, or renal cell carcinoma.

Samples (test samples) may be obtained from any source. In some instances, the source is a human. In some instances, the source is a human (or patient) suspected of having a disease or condition. In some instances, the test sample comprises a liquid biopsy. In some instances, the test sample comprises circulating tumor DNA (ctDNA). In some instances, the test sample comprises circulating tumor DNA (ctDNA). In some instances, the test sample is obtained from blood. In some instances, the test sample is substantially cell-free. In some instances, more than one test sample is analyzed sequentially or in parallel. In some instances, at least 1, 2, 3, 4, 5, 10, 20, 50, 100, 200, 500, 1000, or more than 2000 test samples are analyzed. In some instances, the method further comprises detection of minimal residual disease (MRD). In some instances, the patient is suspected of having a disease or condition. In some instances, the disease or condition is a proliferative disease. In some instances, the disease or condition is cancer. In some instances, the patient was previously treated, is currently treated, or has received a clinical diagnosis for cancer. In some instances, the method further comprises ligating sequencing adapters to at least some polynucleotides in the sample, the library, or both. In some instances, the method further comprises amplifying at least some polynucleotides in the sample, the library, or both. In some instances, if one or more variant sequences are not detected in the library, then results obtained from the at least one sample is discarded or re-analyzed.

### Kits

Provided herein are kits comprising libraries of polynucleotides. In some instances, a kit comprises one or more of a reference standards (controls), wherein the reference standard comprises a sample polynucleotide set and a background set; instructions for use of the kit contents; and packaging to hold and describe the kit contents. In some instances, a kit comprises at least two standards selected from sample polynucleotides having a VAF of 0%, 0.1% 0.25%, 0.5%, 1%, or 2% relative to a wild-type genomic sequence. In some instances, a kit comprises five standards each having a VAF of 0%, 0.1% 0.25%, 0.5%, 1%, or 2% relative to a wild-type genomic sequence. In some instances, kits comprise instructions of use of reference standards with one or more sequencing instruments or other instrument which is configured to measure genomic variants. In some instances, the reference standard is packaged in a buffer. In some instances, the reference standard is packaged in a tube. In some instances, the reference standard is not packaged in a plasma-like format. In some instances, the reference standard comprises 500 ng to 5 micrograms of total DNA.

Provided herein are kits for detecting minimal residual disease (MRD) in a sample. In some instances, the kit comprises a library of plurality of polynucleotides comprising at least one variant associated with minimal residual disease (MRD). In some instances, the kit comprises instructions for use of the kit. In some instances, the kit comprises packaging configured to hold and describe the kit contents. In some instances, the kit further comprises a second library comprising at least one variant associated with minimal residual disease (MRD). In some instances, the second library comprises a different frequency of the at least one variant sequence compared to the library. In some instances, the second library comprises a variant sequence that is different from the library. In some instances, a kit comprises the libraries having a VAF of 0%, 0.001%, 0.005%, 0.01%, 0.05%, 0.1% 0.25%, 0.5%, 1%, or 2% relative to a wild-type genomic sequence. In some instances, a kit comprises five standards each having a VAF of 0%, 0.001%, 0.005%, 0.01%, 0.05%, 0.1% 0.25%, 0.5%, 1%, or 2% relative to a wild-type genomic sequence. In some instances, the one or more libraries of the kit is packaged in a buffer. In some instances, the one or more libraries is packaged in a tube. In some instances, the one or more libraries is not packaged in a plasma-like format. In some instances, the one or more libraries comprises 500 ng to 5 micrograms of total DNA.

### Next Generation Sequencing Applications

Downstream applications of polynucleotide libraries (such as sample polynucleotide sets or reference standards) may include next generation sequencing. For example, enrichment of target sequences with a controlled stoichiometry polynucleotide probe library results in more efficient sequencing. The performance of a polynucleotide library for capturing or hybridizing to targets may be defined by a number of different metrics describing efficiency, accuracy, and precision. For example, Picard metrics comprise variables such as HS library size (the number of unique molecules in the library that correspond to target regions, calculated from read pairs), mean target coverage (the percentage of bases reaching a specific coverage level), depth of coverage (number of reads including a given nucleotide) fold enrichment (sequence reads mapping uniquely to the target/reads mapping to the total sample, multiplied by the total sample length/target length), percent off-bait bases (percent of bases not corresponding to bases of the probes/baits), percent off-target (percent of bases not corresponding to bases of interest), usable bases on target, AT or GC dropout rate, fold 80 base penalty (fold over-coverage needed to raise 80 percent of non-zero targets to the mean coverage level), percent zero coverage targets, PF reads (the number of reads passing a quality filter), percent selected bases (the sum of on-bait bases and near-bait bases divided by the total aligned bases), percent duplication, or other variable consistent with the specification.

Read depth (sequencing depth, or sampling) represents the total number of times a sequenced nucleic acid fragment (a “read”) is obtained for a sequence. Theoretical read depth is defined as the expected number of times the same nucleotide is read, assuming reads are perfectly distributed throughout an idealized genome. Read depth is expressed as function of % coverage (or coverage breadth). For example, 10 million reads of a 1 million base genome, perfectly distributed, theoretically results in 10× read depth of 100% of the sequences. In practice, a greater number of reads (higher theoretical read depth, or oversampling) may be needed to obtain the desired read depth for a percentage of the target sequences. Enrichment of target sequences with a controlled stoichiometry probe library increases the efficiency of downstream sequencing, as fewer total reads will be required to obtain an outcome with an acceptable number of reads over a desired % of target sequences. For example, in some instances 55× theoretical read depth of target sequences results in at least 30× coverage of at least 90% of the sequences. In some instances no more than 55× theoretical read depth of target sequences results in at least 30× read depth of at least 80% of the sequences. In some instances no more than 55× theoretical read depth of target sequences results in at least 30× read depth of at least 95% of the sequences. In some instances no more than 55× theoretical read depth of target sequences results in at least 10× read depth of at least 98% of the sequences. In some instances, 55× theoretical read depth of target sequences results in at least 20× read depth of at least 98% of the sequences. In some instances no more than 55× theoretical read depth of target sequences results in at least 5× read depth of at least 98% of the sequences.

Increasing the concentration of probes during hybridization with targets can lead to an increase in read depth. In some instances, the concentration of probes is increased by at least 1.5×, 2.0×, 2.5×, 3×, 3.5×, 4×, 5×, or more than 5×. In some instances, increasing the probe concentration results in at least a 1000% increase, or a 20%, 30%, 40%, 50%, 60%, 70%, 80%, 90%, 100%, 200%, 300%, 500%, 750%, 1000%, or more than a 1000% increase in read depth. In some instances, increasing the probe concentration by 3× results in a 1000% increase in read depth. In some instances, sequencing is performed to achieve a theoretical read depth of at least 30×, 50×, 100×, 150×, 200×, 250×, 300×, 500×, or at least 1000×. In some instances, sequencing is performed to achieve a theoretical read depth of about 30×, 50×, 100×, 150×, 200×, 250×, 300×, 500×, or about 1000×. In some instances, sequencing is performed to achieve a theoretical read depth of no more than 30×, 50×, 100×, 150×, 200×, 250×, 300×, 500×, or no more than 1000×. In some instances, sequencing is performed to achieve an actual read depth of at least 30×, 50×, 100×, 150×, 200×, 250×, 300×, 500×, or at least 1000×. In some instances, sequencing is performed to achieve an actual read depth of no more than 30×, 50×, 100×, 150×, 200×, 250×, 300×, 500×, or no more than 1000×. In some instances, sequencing is performed to achieve an actual read depth of about 30×, 50×, 100×, 150×, 200×, 250×, 300×, 500×, or about 1000×.

On-target rate represents the percentage of sequencing reads that correspond with the desired target sequences. In some instances, a controlled stoichiometry polynucleotide probe library results in an on-target rate of at least 30%, or at least 35%, 40%, 45%, 50%, 55%, 60%, 65%, 70%, 75%, 80%, 85%, or at least 90%. Increasing the concentration of polynucleotide probes during contact with target nucleic acids leads to an increase in the on-target rate. In some instances, the concentration of probes is increased by at least 1.5×, 2.0×, 2.5×, 3×, 3.5×, 4×, 5×, or more than 5×. In some instances, increasing the probe concentration results in at least a 20% increase, or a 10%, 20%, 30%, 40%, 50%, 60%, 70%, 80%, 90%, 100%, 200%, 300%, or at least a 500% increase in on-target binding. In some instances, increasing the probe concentration by 3× results in a 20% increase in on-target rate.

Coverage uniformity is in some cases calculated as the read depth as a function of the target sequence identity. Higher coverage uniformity results in a lower number of sequencing reads needed to obtain the desired read depth. For example, a property of the target sequence may affect the read depth, for example, high or low GC or AT content, repeating sequences, trailing adenines, secondary structure, affinity for target sequence binding (for amplification, enrichment, or detection), stability, melting temperature, biological activity, ability to assemble into larger fragments, sequences containing modified nucleotides or nucleotide analogues, or any other property of polynucleotides. Enrichment of target sequences with controlled stoichiometry polynucleotide probe libraries results in higher coverage uniformity after sequencing. In some instances, 95% of the sequences have a read depth that is within 1× of the mean library read depth, or about 0.05, 0.1, 0.2, 0.5, 0.7, 1, 1.2, 1.5, 1.7 or about within 2× the mean library read depth. In some instances, 80%, 85%, 90%, 95%, 97%, or 99% of the sequences have a read depth that is within 1× of the mean.

Enrichment of Target Nucleic Acids with a Polynucleotide Probe Library

A probe library described herein may be used to enrich target polynucleotides present in a population of sample polynucleotides, for a variety of downstream applications. In one some instances, a sample is obtained from one or more sources, and the population of sample polynucleotides is isolated. Samples are obtained (by way of non-limiting example) from biological sources such as saliva, blood, tissue, skin, or completely synthetic sources. The plurality of polynucleotides obtained from the sample are fragmented, end-repaired, and adenylated to form a double stranded sample nucleic acid fragment. In some instances, end repair is accomplished by treatment with one or more enzymes, such as T4 DNA polymerase, klenow enzyme, and T4 polynucleotide kinase in an appropriate buffer. A nucleotide overhang to facilitate ligation to adapters is added, in some instances with 3′ to 5′ exo minus klenow fragment and dATP.

Adapters (such as universal adapters) may be ligated to both ends of the sample polynucleotide fragments with a ligase, such as T4 ligase, to produce a library of adapter-tagged polynucleotide strands, and the adapter-tagged polynucleotide library is amplified with primers, such as universal primers. In some instances, the adapters are Y-shaped adapters comprising one or more primer binding sites, one or more grafting regions, and one or more index (or barcode) regions. In some instances, the one or more index region is present on each strand of the adapter. In some instances, grafting regions are complementary to a flow cell surface, and facilitate next generation sequencing of sample libraries. In some instances, Y-shaped adapters comprise partially complementary sequences. In some instances, Y-shaped adapters comprise a single thymidine overhang which hybridizes to the overhanging adenine of the double stranded adapter-tagged polynucleotide strands. Y-shaped adapters may comprise modified nucleic acids, that are resistant to cleavage. For example, a phosphorothioate backbone is used to attach an overhanging thymidine to the 3′ end of the adapters. If universal primers are used, amplification of the library is performed to add barcoded primers to the adapters. A library of double stranded adapter-tagged polynucleotide strands is contacted with polynucleotide probes, to form hybrid pairs. Such pairs are separated from un-hybridized fragments, and isolated from probes to produce an enriched library. The enriched library may then be sequenced.

The library of double stranded sample nucleic acid fragments is then denatured in the presence of adapter blockers. Adapter blockers minimize off-target hybridization of probes to the adapter sequences (instead of target sequences) present on the adapter-tagged polynucleotide strands, and/or prevent intermolecular hybridization of adapters (i.e., “daisy chaining”). Denaturation is carried out in some instances at 96° C., or at about 85, 87, 90, 92, 95, 97, 98 or about 99° C. A polynucleotide targeting library (probe library) is denatured in a hybridization solution, in some instances at 96° C., at about 85, 87, 90, 92, 95, 97, 98 or 99° C. The denatured adapter-tagged polynucleotide library and the hybridization solution are incubated for a suitable amount of time and at a suitable temperature to allow the probes to hybridize with their complementary target sequences. In some instances, a suitable hybridization temperature is about 45 to 80° C., or at least 45, 50, 55, 60, 65, 70, 75, 80, 85, or 90° C. In some instances, the hybridization temperature is 70° C. In some instances, a suitable hybridization time is 16 hours, or at least 4, 6, 8, 10, 12, 14, 16, 18, 20, 22, or more than 22 hours, or about 12 to 20 hours. Binding buffer is then added to the hybridized adapter-tagged-polynucleotide probes, and a solid support comprising a capture moiety is used to selectively bind the hybridized adapter-tagged polynucleotide-probes. The solid support is washed with buffer to remove unbound polynucleotides before an elution buffer is added to release the enriched, tagged polynucleotide fragments from the solid support. In some instances, the solid support is washed 2 times, or 1, 2, 3, 4, 5, or 6 times. The enriched library of adapter-tagged polynucleotide fragments is amplified and the enriched library is sequenced.

A plurality of nucleic acids (i.e., genomic sequence) may obtained from a sample, and fragmented, optionally end-repaired, and adenylated. Adapters are ligated to both ends of the polynucleotide fragments to produce a library of adapter-tagged polynucleotide strands, and the adapter-tagged polynucleotide library is amplified. The adapter-tagged polynucleotide library is then denatured at high temperature, preferably 96° C., in the presence of adapter blockers. A polynucleotide targeting library (probe library) is denatured in a hybridization solution at high temperature, preferably about 90 to 99° C., and combined with the denatured, tagged polynucleotide library in hybridization solution for about 10 to 24 hours at about 45 to 80° C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 2 and 5 times to remove unbound polynucleotides before an elution buffer is added to release the enriched, adapter-tagged polynucleotide fragments from the solid support. The enriched library of adapter-tagged polynucleotide fragments is amplified and then the library is sequenced. Alternative variables such as incubation times, temperatures, reaction volumes/concentrations, number of washes, or other variables consistent with the specification are also employed in the method.

In any of the instances, the detection or quantification analysis of the oligonucleotides can be accomplished by sequencing. The subunits or entire synthesized oligonucleotides can be detected via full sequencing of all oligonucleotides by any suitable methods known in the art, e.g., Illumina sequencing by synthesis, PacBio nanopore sequencing, or BGI/MGI nanoball sequencing, including the sequencing methods described herein.

Sequencing can be accomplished through classic Sanger sequencing methods which are well known in the art. Sequencing can also be accomplished using high-throughput systems some of which allow detection of a sequenced nucleotide immediately after or upon its incorporation into a growing strand, i.e., detection of sequence in red time or substantially real time. In some cases, high throughput sequencing generates at least 1,000, at least 5,000, at least 10,000, at least 20,000, at least 30,000, at least 40,000, at least 50,000, at least 100,000 or at least 500,000 sequence reads per hour; with each read being at least 50, at least 60, at least 70, at least 80, at least 90, at least 100, at least 120 or at least 150 bases per read.

In some instances, high-throughput sequencing involves the use of technology available by Illumina's Genome Analyzer IIX, MiSeq personal sequencer, or HiSeq systems, such as those using HiSeq 2500, HiSeq 1500, HiSeq 2000, HiSeq 1000, iSeq 100, Mini Seq, MiSeq, NextSeq 550, NextSeq 2000, NextSeq 550, or NovaSeq 6000. These machines use reversible terminator-based sequencing by synthesis chemistry. These machines can generate 6000 Gb or more reads in 13-44 hours. Smaller systems may be utilized for runs within 3, 2, 1 days or less time. Short synthesis cycles may be used to minimize the time it takes to obtain sequencing results.

In some instances, high-throughput sequencing involves the use of technology available by ABI Solid System. This genetic analysis platform that enables massively parallel sequencing of clonally-amplified DNA fragments linked to beads. The sequencing methodology is based on sequential ligation with dye-labeled oligonucleotides.

The next generation sequencing can comprise ion semiconductor sequencing (e.g., using technology from Life Technologies (Ion Torrent)). Ion semiconductor sequencing can take advantage of the fact that when a nucleotide is incorporated into a strand of DNA, an ion can be released. To perform ion semiconductor sequencing, a high density array of micromachined wells can be formed. Each well can hold a single DNA template. Beneath the well can be an ion sensitive layer, and beneath the ion sensitive layer can be an ion sensor. When a nucleotide is added to a DNA, H+ can be released, which can be measured as a change in pH. The H+ ion can be converted to voltage and recorded by the semiconductor sensor. An array chip can be sequentially flooded with one nucleotide after another. No scanning, light, or cameras can be required. In some cases, an IONPROTON™ Sequencer is used to sequence nucleic acid. In some cases, an IONPGM™ Sequencer is used. The Ion Torrent Personal Genome Machine (PGM) can do 10 million reads in two hours.

In some instances, high-throughput sequencing involves the use of technology available by Helicos BioSciences Corporation (Cambridge, Mass.) such as the Single Molecule Sequencing by Synthesis (SMSS) method. SMSS is unique because it allows for sequencing the entire human genome in up to 24 hours. Finally, SMSS is powerful because, like the MW technology, it does not require a pre amplification step prior to hybridization. In fact, SMSS does not require any amplification.

In some instances, high-throughput sequencing involves the use of technology available by 454 Lifesciences, Inc. (Branford, Conn.) such as the Pico Titer Plate device which includes a fiber optic plate that transmits chemiluminescent signal generated by the sequencing reaction to be recorded by a CCD camera in the instrument. This use of fiber optics allows for the detection of a minimum of 20 million base pairs in 4.5 hours.

Methods for using bead amplification followed by fiber optics detection may comprise methods described in Marguiles et al., “Genome sequencing in microfabricated high-density picolitre reactors”, Nature, 2005, vol. 437, pages 376-380.

In some instances, high-throughput sequencing is performed using Clonal Single Molecule Array (Solexa, Inc.) or sequencing-by-synthesis (SBS) utilizing reversible terminator chemistry, such as described in Constans, “Beyond Sanger: toward the $1,000 genome: new technologies promise faster and cheaper whole-genome sequencing”, The Scientist, 2003, vol. 17, issue 13, page 36+. High-throughput sequencing of oligonucleotides can be achieved using any suitable sequencing method known in the art, such as those commercialized by Pacific Biosciences, Complete Genomics, Genia Technologies, Halcyon Molecular, Oxford Nanopore Technologies and the like. Overall such systems involve sequencing a target oligonucleotide molecule having a plurality of bases by the temporal addition of bases via a polymerization reaction that is measured on a molecule of oligonucleotide, i.e., the activity of a nucleic acid polymerizing enzyme on the template oligonucleotide molecule to be sequenced is followed in real time. Sequence can then be deduced by identifying which base is being incorporated into the growing complementary strand of the target oligonucleotide by the catalytic activity of the nucleic acid polymerizing enzyme at each step in the sequence of base additions. A polymerase on the target oligonucleotide molecule complex is provided in a position suitable to move along the target oligonucleotide molecule and extend the oligonucleotide primer at an active site. A plurality of labeled types of nucleotide analogs are provided proximate to the active site, with each distinguishably type of nucleotide analog being complementary to a different nucleotide in the target oligonucleotide sequence. The growing oligonucleotide strand is extended by using the polymerase to add a nucleotide analog to the oligonucleotide strand at the active site, where the nucleotide analog being added is complementary to the nucleotide of the target oligonucleotide at the active site. The nucleotide analog added to the oligonucleotide primer as a result of the polymerizing step is identified. The steps of providing labeled nucleotide analogs, polymerizing the growing oligonucleotide strand, and identifying the added nucleotide analog are repeated so that the oligonucleotide strand is further extended and the sequence of the target oligonucleotide is determined.

The next generation sequencing technique can comprises real-time (SMRT™) technology by Pacific Biosciences. In SMRT, each of four DNA bases can be attached to one of four different fluorescent dyes. These dyes can be phospho linked. A single DNA polymerase can be immobilized with a single molecule of template single stranded DNA at the bottom of a zero-mode waveguide (ZMW). A ZMW can be a confinement structure which enables observation of incorporation of a single nucleotide by DNA polymerase against the background of fluorescent nucleotides that can rapidly diffuse in an out of the ZMW (in microseconds). It can take several milliseconds to incorporate a nucleotide into a growing strand. During this time, the fluorescent label can be excited and produce a fluorescent signal, and the fluorescent tag can be cleaved off. The ZMW can be illuminated from below. Attenuated light from an excitation beam can penetrate the lower 20-30 nm of each ZMW. A microscope with a detection limit of 20 zepto liters (10″ liters) can be created. The tiny detection volume can provide 1000-fold improvement in the reduction of background noise. Detection of the corresponding fluorescence of the dye can indicate which base was incorporated. The process can be repeated.

In some cases, the next generation sequencing is nanopore sequencing. See, e.g., Soni et al., “Progress toward ultrafast DNA sequencing using solid-state nanopores”, Clin Chem., 2007, vol. 53, pages 1996-2001. A nanopore can be a small hole, of the order of about one nanometer in diameter. Immersion of a nanopore in a conducting fluid and application of a potential across it can result in a slight electrical current due to conduction of ions through the nanopore. The amount of current which flows can be sensitive to the size of the nanopore. As a DNA molecule passes through a nanopore, each nucleotide on the DNA molecule can obstruct the nanopore to a different degree. Thus, the change in the current passing through the nanopore as the DNA molecule passes through the nanopore can represent a reading of the DNA sequence. The nanopore sequencing technology can be from Oxford Nanopore Technologies, e.g., a GridION system. A single nanopore can be inserted in a polymer membrane across the top of a microwell. Each microwell can have an electrode for individual sensing. The microwells can be fabricated into an array chip, with 100,000 or more microwells (e.g., more than 200,000, 300,000, 400,000, 500,000, 600,000, 700,000, 800,000, 900,000, or 1,000,000) per chip. An instrument (or node) can be used to analyze the chip. Data can be analyzed in real-time. One or more instruments can be operated at a time. The nanopore can be a protein nanopore, e.g., the protein alpha-hemolysin, a heptameric protein pore. The nanopore can be a solid-state nanopore made, e.g., a nanometer sized hole formed in a synthetic membrane (e.g., SiNₓ, or SiO₂). The nanopore can be a hybrid pore (e.g., an integration of a protein pore into a solid-state membrane). The nanopore can be a nanopore with an integrated sensors (e.g., tunneling electrode detectors, capacitive detectors, or graphene based nano-gap or edge state detectors (see e.g., Garaj et al., “Graphene as a subnanometre trans-electrode membrane, Nature, 2010, vol. 67, pages 190-193). A nanopore can be functionalized for analyzing a specific type of molecule (e.g., DNA, RNA, or protein). Nanopore sequencing can comprise “strand sequencing” in which intact DNA polymers can be passed through a protein nanopore with sequencing in real time as the DNA translocates the pore. An enzyme can separate strands of a double stranded DNA and feed a strand through a nanopore. The DNA can have a hairpin at one end, and the system can read both strands. In some cases, nanopore sequencing is “exonuclease sequencing” in which individual nucleotides can be cleaved from a DNA strand by a processive exonuclease, and the nucleotides can be passed through a protein nanopore. The nucleotides can transiently bind to a molecule in the pore (e.g., cyclodextran). A characteristic disruption in current can be used to identify bases.

Nanopore sequencing technology from GENIA can be used. An engineered protein pore can be embedded in a lipid bilayer membrane. “Active Control” technology can be used to enable efficient nanopore-membrane assembly and control of DNA movement through the channel. In some cases, the nanopore sequencing technology is from NABsys. Genomic DNA can be fragmented into strands of average length of about 100 kb. The 100 kb fragments can be made single stranded and subsequently hybridized with a 6-mer probe. The genomic fragments with probes can be driven through a nanopore, which can create a current-versus-time tracing. The current tracing can provide the positions of the probes on each genomic fragment. The genomic fragments can be lined up to create a probe map for the genome. The process can be done in parallel for a library of probes. A genome-length probe map for each probe can be generated. Errors can be fixed with a process termed “moving window Sequencing By Hybridization (mwSBH).” In some cases, the nanopore sequencing technology is from IBM/Roche. An electron beam can be used to make a nanopore sized opening in a microchip. An electrical field can be used to pull or thread DNA through the nanopore. A DNA transistor device in the nanopore can comprise alternating nanometer sized layers of metal and dielectric. Discrete charges in the DNA backbone can get trapped by electrical fields inside the DNA nanopore. Turning off and on gate voltages can allow the DNA sequence to be read.

The next generation sequencing can comprise DNA nanoball sequencing (as performed, e.g., by Complete Genomics; see e.g., Drmanac et al., “Human genome sequencing using unchained base reads on self-assembling DNA nanoarrays”, Science, 2010, vol. 327, pages 78-81). DNA can be isolated, fragmented, and size selected. For example, DNA can be fragmented (e.g., by sonication) to a mean length of about 500 bp. Adaptors (Adl) can be attached to the ends of the fragments. The adaptors can be used to hybridize to anchors for sequencing reactions. DNA with adaptors bound to each end can be PCR amplified. The adaptor sequences can be modified so that complementary single strand ends bind to each other forming circular DNA. The DNA can be methylated to protect it from cleavage by a type IIS restriction enzyme used in a subsequent step. An adaptor (e.g., the right adaptor) can have a restriction recognition site, and the restriction recognition site can remain non-methylated. The non-methylated restriction recognition site in the adaptor can be recognized by a restriction enzyme (e.g., Acul), and the DNA can be cleaved by Acul 13 bp to the right of the right adaptor to form linear double stranded DNA. A second round of right and left adaptors (Ad2) can be ligated onto either end of the linear DNA, and all DNA with both adapters bound can be PCR amplified (e.g., by PCR). Ad2 sequences can be modified to allow them to bind each other and form circular DNA. The DNA can be methylated, but a restriction enzyme recognition site can remain non-methylated on the left Adl adapter. A restriction enzyme (e.g., Acul) can be applied, and the DNA can be cleaved 13 bp to the left of the Adl to form a linear DNA fragment. A third round of right and left adaptor (Ad3) can be ligated to the right and left flank of the linear DNA, and the resulting fragment can be PCR amplified. The adaptors can be modified so that they can bind to each other and form circular DNA. A type III restriction enzyme (e.g., EcoP15) can be added; EcoP15 can cleave the DNA 26 bp to the left of Ad3 and 26 bp to the right of Ad2. This cleavage can remove a large segment of DNA and linearize the DNA once again. A fourth round of right and left adaptors (Ad4) can be ligated to the DNA, the DNA can be amplified (e.g., by PCR), and modified so that they bind each other and form the completed circular DNA template.

Rolling circle replication (e.g., using Phi 29 DNA polymerase) can be used to amplify small fragments of DNA. The four adaptor sequences can contain palindromic sequences that can hybridize and a single strand can fold onto itself to form a DNA nanoball (DNB™) which can be approximately 200-300 nanometers in diameter on average. A DNA nanoball can be attached (e.g., by adsorption) to a microarray (sequencing flow cell). The flow cell can be a silicon wafer coated with silicon dioxide, titanium and hexamethyldisilazane (HMDS) and a photoresist material. Sequencing can be performed by unchained sequencing by ligating fluorescent probes to the DNA. The color of the fluorescence of an interrogated position can be visualized by a high resolution camera. The identity of nucleotide sequences between adaptor sequences can be determined.

A population of polynucleotides may be enriched prior to adapter ligation. In one example, a plurality of polynucleotides is obtained from a sample, fragmented, optionally end-repaired, and denatured at high temperature, preferably 90-99° C. A polynucleotide targeting library (probe library) is denatured in a hybridization solution at high temperature, preferably about 90 to 99° C., and combined with the denatured, tagged polynucleotide library in hybridization solution for about 10 to 24 hours at about 45 to 80° C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 2 and 5 times to remove unbound polynucleotides before an elution buffer is added to release the enriched, adapter-tagged polynucleotide fragments from the solid support. The enriched polynucleotide fragments are then polyadenylated, adapters are ligated to both ends of the polynucleotide fragments to produce a library of adapter-tagged polynucleotide strands, and the adapter-tagged polynucleotide library is amplified. The adapter-tagged polynucleotide library is then sequenced.

A polynucleotide targeting library may also be used to filter undesired sequences from a plurality of polynucleotides, by hybridizing to undesired fragments. For example, a plurality of polynucleotides is obtained from a sample, and fragmented, optionally end-repaired, and adenylated. Adapters are ligated to both ends of the polynucleotide fragments to produce a library of adapter-tagged polynucleotide strands, and the adapter-tagged polynucleotide library is amplified. Alternatively, adenylation and adapter ligation steps are instead performed after enrichment of the sample polynucleotides. The adapter-tagged polynucleotide library is then denatured at high temperature, preferably 90-99° C., in the presence of adapter blockers. A polynucleotide filtering library (probe library) designed to remove undesired, non-target sequences is denatured in a hybridization solution at high temperature, preferably about 90 to 99° C., and combined with the denatured, tagged polynucleotide library in hybridization solution for about 10 to 24 hours at about 45 to 80° C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 1 and 5 times to elute unbound adapter-tagged polynucleotide fragments. The enriched library of unbound adapter-tagged polynucleotide fragments is amplified and then the amplified library is sequenced.

### Highly Parallel De Novo Nucleic Acid Synthesis

Described herein is a platform approach utilizing miniaturization, parallelization, and vertical integration of the end-to-end process from polynucleotide synthesis to gene assembly within Nano wells on silicon to create a revolutionary synthesis platform. Devices described herein provide, with the same footprint as a 96-well plate, a silicon synthesis platform is capable of increasing throughput by a factor of 100 to 1,000 compared to traditional synthesis methods, with production of up to approximately 1,000,000 polynucleotides in a single highly-parallelized run. In some instances, a single silicon plate described herein provides for synthesis of about 6,100 non-identical polynucleotides. In some instances, each of the non-identical polynucleotides is located within a cluster. A cluster may comprise 50 to 500 non-identical polynucleotides.

Methods described herein provide for synthesis of a library of polynucleotides each encoding for a predetermined variant of at least one predetermined reference nucleic acid sequence. In some cases, the predetermined reference sequence is nucleic acid sequence encoding for a protein, and the variant library comprises sequences encoding for variation of at least a single codon such that a plurality of different variant sequences of a single residue in the subsequent protein encoded by the synthesized nucleic acid are generated by standard translation processes. The synthesized specific alterations in the nucleic acid sequence can be introduced by incorporating nucleotide changes into overlapping or blunt ended polynucleotide primers. Alternatively, a population of polynucleotides may collectively encode for a long nucleic acid (e.g., a gene) and variants thereof. In this arrangement, the population of polynucleotides can be hybridized and subject to standard molecular biology techniques to form the long nucleic acid (e.g., a gene) and variants thereof. When the long nucleic acid (e.g., a gene) and variants thereof are expressed in cells, a variant protein library is generated. Similarly, provided here are methods for synthesis of variant libraries encoding for RNA sequences (e.g., miRNA, shRNA, and mRNA) or DNA sequences (e.g., enhancer, promoter, UTR, and terminator regions). Also provided here are downstream applications for variant sequences selected out of the libraries synthesized using methods described here. Downstream applications include identification of variant nucleic acid or protein sequences with enhanced biologically relevant functions, e.g., biochemical affinity, enzymatic activity, changes in cellular activity, and for the treatment or prevention of a disease state.

### Substrates

Provided herein are substrates comprising a plurality of clusters, wherein each cluster comprises a plurality of loci that support the attachment and synthesis of polynucleotides. The term “locus” as used herein refers to a discrete region on a structure which provides support for polynucleotides encoding for a single predetermined sequence to extend from the surface. In some instances, a locus is on a two dimensional surface, e.g., a substantially planar surface. In some instances, a locus refers to a discrete raised or lowered site on a surface e.g., a well, micro well, channel, or post. In some instances, a surface of a locus comprises a material that is actively functionalized to attach to at least one nucleotide for polynucleotide synthesis, or preferably, a population of identical nucleotides for synthesis of a population of polynucleotides. In some instances, polynucleotide refers to a population of polynucleotides encoding for the same nucleic acid sequence. In some instances, a surface of a device is inclusive of one or a plurality of surfaces of a substrate.

Provided herein are structures that may comprise a surface that supports the synthesis of a plurality of polynucleotides having different predetermined sequences at addressable locations on a common support. In some instances, a device provides support for the synthesis of more than 2,000; 5,000; 10,000; 20,000; 30,000; 50,000; 75,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical polynucleotides. In some instances, the device provides support for the synthesis of more than 2,000; 5,000; 10,000; 20,000; 30,000; 50,000; 75,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more polynucleotides encoding for distinct sequences. In some instances, at least a portion of the polynucleotides have an identical sequence or are configured to be synthesized with an identical sequence.

Provided herein are methods and devices for manufacture and growth of polynucleotides about 5, 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, or 2000 bases in length. In some instances, the length of the polynucleotide formed is about 5, 10, 20, 30, 40, 50, 60, 70, 80, 90, 100, 125, 150, 175, 200, or 225 bases in length. A polynucleotide may be at least 5, 10, 20, 30, 40, 50, 60, 70, 80, 90, or 100 bases in length. A polynucleotide may be from 10 to 225 bases in length, from 12 to 100 bases in length, from 20 to 150 bases in length, from 20 to 130 bases in length, or from 30 to 100 bases in length.

In some instances, polynucleotides are synthesized on distinct loci of a substrate, wherein each locus supports the synthesis of a population of polynucleotides. In some instances, each locus supports the synthesis of a population of polynucleotides having a different sequence than a population of polynucleotides grown on another locus. In some instances, the loci of a device are located within a plurality of clusters. In some instances, a device comprises at least 10, 500, 1000, 2000, 3000, 4000, 5000, 6000, 7000, 8000, 9000, 10000, 11000, 12000, 13000, 14000, 15000, 20000, 30000, 40000, 50000 or more clusters. In some instances, a device comprises more than 2,000; 5,000; 10,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; or 10,000,000 or more distinct loci. In some instances, a device comprises about 10,000 distinct loci. The amount of loci within a single cluster is varied in different instances. In some instances, each cluster includes 1, 2, 3, 4, 5, 6, 7, 8, 9, 10, 20, 30, 40, 50, 60, 70, 80, 90, 100, 120, 130, 150, 200, 300, 400, 500, 1000 or more loci. In some instances, each cluster includes about 50-500 loci. In some instances, each cluster includes about 100-200 loci. In some instances, each cluster includes about 100-150 loci. In some instances, each cluster includes about 109, 121, 130 or 137 loci. In some instances, each cluster includes about 19, 20, 61, 64 or more loci.

The number of distinct polynucleotides synthesized on a device may be dependent on the number of distinct loci available in the substrate. In some instances, the density of loci within a cluster of a device is at least or about 1 locus per mm², 10 loci per mm², 25 loci per mm², 50 loci per mm², 65 loci per mm², 75 loci per mm², 100 loci per mm², 130 loci per mm², 150 loci per mm², 175 loci per mm², 200 loci per mm², 300 loci per mm², 400 loci per mm², 500 loci per mm², 1,000 loci per mm² or more. In some instances, a device comprises from about 10 loci per mm² to about 500 mm², from about 25 loci per mm² to about 400 mm², from about 50 loci per mm² to about 500 mm², from about 100 loci per mm² to about 500 mm², from about 150 loci per mm² to about 500 mm², from about 10 loci per mm² to about 250 mm², from about 50 loci per mm² to about 250 mm², from about 10 loci per mm² to about 200 mm², or from about 50 loci per mm² to about 200 mm². In some instances, the distance from the centers of two adjacent loci within a cluster is from about 10 μm to about 500 μm, from about 10 μm to about 200 μm, or from about 10 μm to about 100 μm. In some instances, the distance from two centers of adjacent loci is greater than about 10 μm, 20 μm, 30 μm, 40 μm, 50 μm, 60 μm, 70 μm, 80 μm, 90 μm or 100 μm. In some instances, the distance from the centers of two adjacent loci is less than about 200 μm, 150 μm, 100 μm, 80 μm, 70 μm, 60 μm, 50 μm, 40 μm, 30 μm, 20 μm or 10 μm. In some instances, each locus has a width of about 0.5 μm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm or 100 μm. In some instances, each locus is has a width of about 0.5 um to 100 μm, about 0.5 μm to 50 μm, about 10 μm to 75 μm, or about 0.5 μm to 50 μm.

In some instances, the density of clusters within a device is at least or about 1 cluster per 100 mm², 1 cluster per 10 mm², 1 cluster per 5 mm², 1 cluster per 4 mm², 1 cluster per 3 mm², 1 cluster per 2 mm², 1 cluster per 1 mm², 2 clusters per 1 mm², 3 clusters per 1 mm², 4 clusters per 1 mm², 5 clusters per 1 mm², 10 clusters per 1 mm², 50 clusters per 1 mm² or more. In some instances, a device comprises from about 1 cluster per 10 mm² to about 10 clusters per 1 mm². In some instances, the distance from the centers of two adjacent clusters is less than about 50 μm, 100 μm, 200 μm, 500 μm, 1000 μm, or 2000 μm or 5000 μm. In some instances, the distance from the centers of two adjacent clusters is from about 50 μm and about 100 μm, from about 50 μm and about 200 μm, from about 50 μm and about 300 μm, from about 50 μm and about 500 μm, and from about 100 μm to about 2000 μm. In some instances, the distance from the centers of two adjacent clusters is from about 0.05 mm to about 50 mm, from about 0.05 mm to about 10 mm, from about 0.05 mm and about 5 mm, from about 0.05 mm and about 4 mm, from about 0.05 mm and about 3 mm, from about 0.05 mm and about 2 mm, from about 0.1 mm and 10 mm, from about 0.2 mm and 10 mm, from about 0.3 mm and about 10 mm, from about 0.4 mm and about 10 mm, from about 0.5 mm and 10 mm, from about 0.5 mm and about 5 mm, or from about 0.5 mm and about 2 mm. In some instances, each cluster has a diameter or width along one dimension of about 0.5 to 2 mm, about 0.5 to 1 mm, or about 1 to 2 mm. In some instances, each cluster has a diameter or width along one dimension of about 0.5, 0.6, 0.7, 0.8, 0.9, 1, 1.1, 1.2, 1.3, 1.4, 1.5, 1.6, 1.7, 1.8, 1.9 or 2 mm. In some instances, each cluster has an interior diameter or width along one dimension of about 0.5, 0.6, 0.7, 0.8, 0.9, 1, 1.1, 1.15, 1.2, 1.3, 1.4, 1.5, 1.6, 1.7, 1.8, 1.9 or 2 mm.

A device may be about the size of a standard 96 well plate, for example from about 100 and 200 mm by from about 50 and 150 mm. In some instances, a device has a diameter less than or equal to about 1000 mm, 500 mm, 450 mm, 400 mm, 300 mm, 250 nm, 200 mm, 150 mm, 100 mm or 50 mm. In some instances, the diameter of a device is from about 25 mm and 1000 mm, from about 25 mm and about 800 mm, from about 25 mm and about 600 mm, from about 25 mm and about 500 mm, from about 25 mm and about 400 mm, from about 25 mm and about 300 mm, or from about 25 mm and about 200. Non-limiting examples of device size include about 300 mm, 200 mm, 150 mm, 130 mm, 100 mm, 76 mm, 51 mm and 25 mm. In some instances, a device has a planar surface area of at least about 100 mm²; 200 mm²; 500 mm²; 1,000 mm²; 2,000 mm²; 5,000 mm²; 10,000 mm²; 12,000 mm²; 15,000 mm²; 20,000 mm²; 30,000 mm²; 40,000 mm²; 50,000 mm² or more. In some instances, the thickness of a device is from about 50 mm and about 2000 mm, from about 50 mm and about 1000 mm, from about 100 mm and about 1000 mm, from about 200 mm and about 1000 mm, or from about 250 mm and about 1000 mm. Non-limiting examples of device thickness include 275 mm, 375 mm, 525 mm, 625 mm, 675 mm, 725 mm, 775 mm and 925 mm. In some instances, the thickness of a device varies with diameter and depends on the composition of the substrate. For example, a device comprising materials other than silicon has a different thickness than a silicon device of the same diameter. Device thickness may be determined by the mechanical strength of the material used and the device must be thick enough to support its own weight without cracking during handling. In some instances, a structure comprises a plurality of devices described herein.

### Surface Materials

Provided herein is a device comprising a surface, wherein the surface is modified to support polynucleotide synthesis at predetermined locations and with a resulting low error rate, a low dropout rate, a high yield, and a high oligo representation. In some instances, surfaces of a device for polynucleotide synthesis provided herein are fabricated from a variety of materials capable of modification to support a de novo polynucleotide synthesis reaction. In some cases, the devices are sufficiently conductive, e.g., are able to form uniform electric fields across all or a portion of the device. A device described herein may comprise a flexible material. Exemplary flexible materials include, without limitation, modified nylon, unmodified nylon, nitrocellulose, and polypropylene. A device described herein may comprise a rigid material. Exemplary rigid materials include, without limitation, glass, fuse silica, silicon, silicon dioxide, silicon nitride, plastics (for example, polytetrafluoroethylene, polypropylene, polystyrene, polycarbonate, and blends thereof, and metals (for example, gold, platinum). Device disclosed herein may be fabricated from a material comprising silicon, polystyrene, agarose, dextran, cellulosic polymers, polyacrylamides, polydimethylsiloxane (PDMS), glass, or any combination thereof. In some cases, a device disclosed herein is manufactured with a combination of materials listed herein or any other suitable material known in the art.

A listing of tensile strengths for exemplary materials described herein is provides as follows: nylon (70 MPa), nitrocellulose (1.5 MPa), polypropylene (40 MPa), silicon (268 MPa), polystyrene (40 MPa), agarose (1-10 MPa), polyacrylamide (1-10 MPa), polydimethylsiloxane (PDMS) (3.9-10.8 MPa). Solid supports described herein can have a tensile strength from 1 to 300, 1 to 40, 1 to 10, 1 to 5, or 3 to 11 MPa. Solid supports described herein can have a tensile strength of about 1, 1.5, 2, 3, 4, 5, 6, 7, 8, 9, 10, 11, 20, 25, 40, 50, 60, 70, 80, 90, 100, 150, 200, 250, 270, or more MPa. In some instances, a device described herein comprises a solid support for polynucleotide synthesis that is in the form of a flexible material capable of being stored in a continuous loop or reel, such as a tape or flexible sheet.

Young's modulus measures the resistance of a material to elastic (recoverable) deformation under load. A listing of Young's modulus for stiffness of exemplary materials described herein is provides as follows: nylon (3 GPa), nitrocellulose (1.5 GPa), polypropylene (2 GPa), silicon (150 GPa), polystyrene (3 GPa), agarose (1-10 GPa), polyacrylamide (1-10 GPa), polydimethylsiloxane (PDMS) (1-10 GPa). Solid supports described herein can have a Young's moduli from 1 to 500, 1 to 40, 1 to 10, 1 to 5, or 3 to 11 GPa. Solid supports described herein can have a Young's moduli of about 1, 1.5, 2, 3, 4, 5, 6, 7, 8, 9, 10, 11, 20, 25, 40, 50, 60, 70, 80, 90, 100, 150, 200, 250, 400, 500 GPa, or more. As the relationship between flexibility and stiffness are inverse to each other, a flexible material has a low Young's modulus and changes its shape considerably under load.

In some cases, a device disclosed herein comprises a silicon dioxide base and a surface layer of silicon oxide. Alternatively, the device may have a base of silicon oxide. Surface of the device provided here may be textured, resulting in an increase overall surface area for polynucleotide synthesis. Device disclosed herein may comprise at least 5%, 10%, 25%, 50%, 80%, 90%, 95%, or 99% silicon. A device disclosed herein may be fabricated from a silicon on insulator (SOI) wafer.

### Surface Architecture

Provided herein are devices comprising raised and/or lowered features. One benefit of having such features is an increase in surface area to support polynucleotide synthesis. In some instances, a device having raised and/or lowered features is referred to as a three-dimensional substrate. In some instances, a three-dimensional device comprises one or more channels. In some instances, one or more loci comprise a channel. In some instances, the channels are accessible to reagent deposition via a deposition device such as a polynucleotide synthesizer. In some instances, reagents and/or fluids collect in a larger well in fluid communication one or more channels. For example, a device comprises a plurality of channels corresponding to a plurality of loci with a cluster, and the plurality of channels are in fluid communication with one well of the cluster. In some methods, a library of polynucleotides is synthesized in a plurality of loci of a cluster.

In some instances, the structure is configured to allow for controlled flow and mass transfer paths for polynucleotide synthesis on a surface. In some instances, the configuration of a device allows for the controlled and even distribution of mass transfer paths, chemical exposure times, and/or wash efficacy during polynucleotide synthesis. In some instances, the configuration of a device allows for increased sweep efficiency, for example by providing sufficient volume for a growing a polynucleotide such that the excluded volume by the growing polynucleotide does not take up more than 50, 45, 40, 35, 30, 25, 20, 15, 14, 13, 12, 11, 10, 9, 8, 7, 6, 5, 4, 3, 2, 1%, or less of the initially available volume that is available or suitable for growing the polynucleotide. In some instances, a three-dimensional structure allows for managed flow of fluid to allow for the rapid exchange of chemical exposure.

Provided herein are methods to synthesize an amount of DNA of 1 fM, 5 fM, 10 fM, 25 fM, 50 fM, 75 fM, 100 fM, 200 fM, 300 fM, 400 fM, 500 fM, 600 fM, 700 fM, 800 fM, 900 fM, 1 μM, 5 μM, 10 pM, 25 μM, 50 μM, 75 μM, 100 μM, 200 μM, 300 μM, 400 μM, 500 μM, 600 μM, 700 μM, 800 μM, 900 pM, or more. In some instances, a polynucleotide library may span the length of about 1%, 2%, 3%, 4%, 5%, 10%, 15%, 20%, 30%, 40%, 50%, 60%, 70%, 80%, 90%, 95%, or 100% of a gene. A gene may be varied up to about 1%, 2%, 3%, 4%, 5%, 10%, 15%, 20%, 30%, 40%, 50%, 60%, 70%, 80%, 85%, 90%, 95%, or 100%.

Non-identical polynucleotides may collectively encode a sequence for at least 1%, 2%, 3%, 4%, 5%, 10%, 15%, 20%, 30%, 40%, 50%, 60%, 70%, 80%, 85%, 90%, 95%, or 100% of a gene. In some instances, a polynucleotide may encode a sequence of 50%, 60%, 70%, 80%, 85%, 90%, 95%, or more of a gene. In some instances, a polynucleotide may encode a sequence of 80%, 85%, 90%, 95%, or more of a gene.

In some instances, segregation is achieved by physical structure. In some instances, segregation is achieved by differential functionalization of the surface generating active and passive regions for polynucleotide synthesis. Differential functionalization is also be achieved by alternating the hydrophobicity across the device surface, thereby creating water contact angle effects that cause beading or wetting of the deposited reagents. Employing larger structures can decrease splashing and cross-contamination of distinct polynucleotide synthesis locations with reagents of the neighboring spots. In some instances, a device, such as a polynucleotide synthesizer, is used to deposit reagents to distinct polynucleotide synthesis locations. Substrates having three-dimensional features are configured in a manner that allows for the synthesis of a large number of polynucleotides (e.g., more than about 10,000) with a low error rate (e.g., less than about 1:500, 1:1000, 1:1500, 1:2,000; 1:3,000; 1:5,000; or 1:10,000). In some instances, a device comprises features with a density of about or greater than about 1, 5, 10, 20, 30, 40, 50, 60, 70, 80, 100, 110, 120, 130, 140, 150, 160, 170, 180, 190, 200, 300, 400 or 500 features per mm².

A well of a device may have the same or different width, height, and/or volume as another well of the substrate. A channel of a device may have the same or different width, height, and/or volume as another channel of the substrate. In some instances, the width of a cluster is from about 0.05 mm to about 50 mm, from about 0.05 mm to about 10 mm, from about 0.05 mm and about 5 mm, from about 0.05 mm and about 4 mm, from about 0.05 mm and about 3 mm, from about 0.05 mm and about 2 mm, from about 0.05 mm and about 1 mm, from about 0.05 mm and about 0.5 mm, from about 0.05 mm and about 0.1 mm, from about 0.1 mm and 10 mm, from about 0.2 mm and 10 mm, from about 0.3 mm and about 10 mm, from about 0.4 mm and about 10 mm, from about 0.5 mm and 10 mm, from about 0.5 mm and about 5 mm, or from about 0.5 mm and about 2 mm. In some instances, the width of a well comprising a cluster is from about 0.05 mm to about 50 mm, from about 0.05 mm to about 10 mm, from about 0.05 mm and about 5 mm, from about 0.05 mm and about 4 mm, from about 0.05 mm and about 3 mm, from about 0.05 mm and about 2 mm, from about 0.05 mm and about 1 mm, from about 0.05 mm and about 0.5 mm, from about 0.05 mm and about 0.1 mm, from about 0.1 mm and 10 mm, from about 0.2 mm and 10 mm, from about 0.3 mm and about 10 mm, from about 0.4 mm and about 10 mm, from about 0.5 mm and 10 mm, from about 0.5 mm and about 5 mm, or from about 0.5 mm and about 2 mm. In some instances, the width of a cluster is less than or about 5 mm, 4 mm, 3 mm, 2 mm, 1 mm, 0.5 mm, 0.1 mm, 0.09 mm, 0.08 mm, 0.07 mm, 0.06 mm or 0.05 mm. In some instances, the width of a cluster is from about 1.0 and 1.3 mm. In some instances, the width of a cluster is about 1.150 mm. In some instances, the width of a well is less than or about 5 mm, 4 mm, 3 mm, 2 mm, 1 mm, 0.5 mm, 0.1 mm, 0.09 mm, 0.08 mm, 0.07 mm, 0.06 mm or 0.05 mm. In some instances, the width of a well is from about 1.0 and 1.3 mm. In some instances, the width of a well is about 1.150 mm. In some instances, the width of a cluster is about 0.08 mm. In some instances, the width of a well is about 0.08 mm. The width of a cluster may refer to clusters within a two-dimensional or three-dimensional substrate.

In some instances, the height of a well is from about 20 μm to about 1000 μm, from about 50 μm to about 1000 μm, from about 100 μm to about 1000 μm, from about 200 μm to about 1000 μm, from about 300 μm to about 1000 μm, from about 400 μm to about 1000 μm, or from about 500 μm to about 1000 μm. In some instances, the height of a well is less than about 1000 μm, less than about 900 μm, less than about 800 μm, less than about 700 μm, or less than about 600 μm.

In some instances, a device comprises a plurality of channels corresponding to a plurality of loci within a cluster, wherein the height or depth of a channel is from about 5 μm to about 500 μm, from about 5 μm to about 400 μm, from about 5 μm to about 300 μm, from about 5 μm to about 200 μm, from about 5 μm to about 100 μm, from about 5 μm to about 50 μm, or from about 10 μm to about 50 μm. In some instances, the height of a channel is less than 100 μm, less than 80 μm, less than 60 μm, less than 40 μm or less than 20 μm.

In some instances, the diameter of a channel, locus (e.g., in a substantially planar substrate) or both channel and locus (e.g., in a three-dimensional device wherein a locus corresponds to a channel) is from about 1 μm to about 1000 μm, from about 1 μm to about 500 μm, from about 1 μm to about 200 μm, from about 1 μm to about 100 μm, from about 5 μm to about 100 μm, or from about 10 μm to about 100 μm, for example, about 90 μm, 80 μm, 70 μm, 60 μm, 50 μm, 40 μm, 30 μm, 20 μm or 10 μm. In some instances, the diameter of a channel, locus, or both channel and locus is less than about 100 μm, 90 μm, 80 μm, 70 μm, 60 μm, 50 μm, 40 μm, 30 μm, 20 μm or 10 μm. In some instances, the distance from the center of two adjacent channels, loci, or channels and loci is from about 1 μm to about 500 μm, from about 1 μm to about 200 μm, from about 1 μm to about 100 μm, from about 5 μm to about 200 μm, from about 5 μm to about 100 μm, from about 5 μm to about 50 μm, or from about 5 μm to about 30 μm, for example, about 20 μm.

### Surface Modifications

In various instances, surface modifications are employed for the chemical and/or physical alteration of a surface by an additive or subtractive process to change one or more chemical and/or physical properties of a device surface or a selected site or region of a device surface. For example, surface modifications include, without limitation, (1) changing the wetting properties of a surface, (2) functionalizing a surface, i.e., providing, modifying or substituting surface functional groups, (3) defunctionalizing a surface, i.e., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface.

In some instances, the addition of a chemical layer on top of a surface (referred to as adhesion promoter) facilitates structured patterning of loci on a surface of a substrate. Exemplary surfaces for application of adhesion promotion include, without limitation, glass, silicon, silicon dioxide and silicon nitride. In some instances, the adhesion promoter is a chemical with a high surface energy. In some instances, a second chemical layer is deposited on a surface of a substrate. In some instances, the second chemical layer has a low surface energy. In some instances, surface energy of a chemical layer coated on a surface supports localization of droplets on the surface. Depending on the patterning arrangement selected, the proximity of loci and/or area of fluid contact at the loci are alterable.

In some instances, a device surface, or resolved loci, onto which nucleic acids or other moieties are deposited, e.g., for polynucleotide synthesis, are smooth or substantially planar (e.g., two-dimensional) or have irregularities, such as raised or lowered features (e.g., three-dimensional features). In some instances, a device surface is modified with one or more different layers of compounds. Such modification layers of interest include, without limitation, inorganic and organic layers such as metals, metal oxides, polymers, small organic molecules and the like. Non-limiting polymeric layers include peptides, proteins, nucleic acids or mimetics thereof (e.g., peptide nucleic acids and the like), polysaccharides, phospholipids, polyurethanes, polyesters, polycarbonates, polyureas, polyamides, polyethyleneamines, polyarylene sulfides, polysiloxanes, polyimides, polyacetates, and any other suitable compounds described herein or otherwise known in the art. In some instances, polymers are heteropolymeric. In some instances, polymers are homopolymeric. In some instances, polymers comprise functional moieties or are conjugated.

In some instances, resolved loci of a device are functionalized with one or more moieties that increase and/or decrease surface energy. In some instances, a moiety is chemically inert. In some instances, a moiety is configured to support a desired chemical reaction, for example, one or more processes in a polynucleotide synthesis reaction. The surface energy, or hydrophobicity, of a surface is a factor for determining the affinity of a nucleotide to attach onto the surface. In some instances, a method for device functionalization may comprise: (a) providing a device having a surface that comprises silicon dioxide; and (b) silanizing the surface using, a suitable silanizing agent described herein or otherwise known in the art, for example, an organofunctional alkoxysilane molecule.

In some instances, the organofunctional alkoxysilane molecule comprises dimethylchloro-octodecyl-silane, methyldichloro-octodecyl-silane, trichloro-octodecyl-silane, trimethyl-octodecyl-silane, triethyl-octodecyl-silane, or any combination thereof. In some instances, a device surface comprises functionalized with polyethylene/polypropylene (functionalized by gamma irradiation or chromic acid oxidation, and reduction to hydroxyalkyl surface), highly crosslinked polystyrene-divinylbenzene (derivatized by chloromethylation, and aminated to benzylamine functional surface), nylon (the terminal aminohexyl groups are directly reactive), or etched with reduced polytetrafluoroethylene. Other methods and functionalizing agents are described in U.S. Pat. No. 5,474,796, which is herein incorporated by reference in its entirety.

In some instances, a device surface is functionalized by contact with a derivatizing composition that contains a mixture of silanes, under reaction conditions effective to couple the silanes to the device surface, typically via reactive hydrophilic moieties present on the device surface. Silanization generally covers a surface through self-assembly with organofunctional alkoxysilane molecules.

A variety of siloxane functionalizing reagents can further be used as currently known in the art, e.g., for lowering or increasing surface energy. The organofunctional alkoxysilanes can be classified according to their organic functions.

Provided herein are devices that may contain patterning of agents capable of coupling to a nucleoside. In some instances, a device may be coated with an active agent. In some instances, a device may be coated with a passive agent. Exemplary active agents for inclusion in coating materials described herein includes, without limitation, N-(3-triethoxysilylpropyl)-4-hydroxybutyramide (HAPS), 11-acetoxyundecyltriethoxysilane, n-decyltriethoxysilane, (3-aminopropyl) trimethoxysilane, (3-aminopropyl)triethoxysilane, 3-glycidoxypropyltrimethoxysilane (GOPS), 3-iodo-propyltrimethoxysilane, butyl-aldehydr-trimethoxysilane, dimeric secondary aminoalkyl siloxanes, (3-aminopropyl)-diethoxy-methylsilane, (3-aminopropyl)-dimethyl-ethoxysilane, and (3-aminopropyl)-trimethoxysilane, (3-glycidoxypropyl)-dimethyl-ethoxysilane, glycidoxy-trimethoxysilane, (3-mercaptopropyl)-trimethoxysilane, 3-4 epoxycyclohexyl-ethyltrimethoxysilane, and (3-mercaptopropyl)-methyl-dimethoxysilane, allyl trichlorochlorosilane, 7-oct-1-enyl trichlorochlorosilane, or bis(3-trimethoxysilylpropyl) amine.

Exemplary passive agents for inclusion in a coating material described herein includes, without limitation, perfluorooctyltrichlorosilane; tridecafluoro-1,1,2,2-tetrahydrooctyl) trichlorosilane; 1H, 1H, 2H, 2H-fluorooctyltriethoxysilane (FOS); trichloro(1H, 1H, 2H, 2H-perfluorooctyl) silane; tert-butyl-[5-fluoro-4-(4,4,5,5-tetramethyl-1,3,2-dioxaborolan-2-yl) indol-1-yl]-dimethyl-silane; CYTOP™; Fluorinert™; perfluoroctyltrichlorosilane (PFOTCS); perfluorooctyldimethylchlorosilane (PFODCS); perfluorodecyltriethoxysilane (PFDTES); pentafluorophenyl-dimethylpropylchloro-silane (PFPTES); perfluorooctyltriethoxysilane; perfluorooctyltrimethoxysilane; octylchlorosilane; dimethylchloro-octodecyl-silane; methyldichloro-octodecyl-silane; trichloro-octodecyl-silane; trimethyl-octodecyl-silane; triethyl-octodecyl-silane; or octadecyltrichlorosilane.

In some instances, a functionalization agent comprises a hydrocarbon silane such as octadecyltrichlorosilane. In some instances, the functionalizing agent comprises 11-acetoxyundecyltriethoxysilane, n-decyltriethoxysilane, (3-aminopropyl) trimethoxysilane, (3-aminopropyl)triethoxysilane, glycidyloxypropyl/trimethoxysilane and N-(3-triethoxysilylpropyl)-4-hydroxybutyramide.

### Polynucleotide Synthesis

Methods of the current disclosure for polynucleotide synthesis may include processes involving phosphoramidite chemistry. In some instances, polynucleotide synthesis comprises coupling a base with phosphoramidite. Polynucleotide synthesis may comprise coupling a base by deposition of phosphoramidite under coupling conditions, wherein the same base is optionally deposited with phosphoramidite more than once, i.e., double coupling. Polynucleotide synthesis may comprise capping of unreacted sites. In some instances, capping is optional. Polynucleotide synthesis may also comprise oxidation or an oxidation step or oxidation steps. Polynucleotide synthesis may comprise deblocking, detritylation, and sulfurization. In some instances, polynucleotide synthesis comprises either oxidation or sulfurization. In some instances, between one or each step during a polynucleotide synthesis reaction, the device is washed, for example, using tetrazole or acetonitrile. Time frames for any one step in a phosphoramidite synthesis method may be less than about 2 minutes, 1 minute, 50 seconds, 40 seconds, 30 seconds, 20 seconds and 10 seconds.

Polynucleotide synthesis using a phosphoramidite method may comprise a subsequent addition of a phosphoramidite building block (e.g., nucleoside phosphoramidite) to a growing polynucleotide chain for the formation of a phosphite triester linkage. Phosphoramidite polynucleotide synthesis proceeds in the 3′ to 5′ direction. Phosphoramidite polynucleotide synthesis allows for the controlled addition of one nucleotide to a growing nucleic acid chain per synthesis cycle. In some instances, each synthesis cycle comprises a coupling step. Phosphoramidite coupling involves the formation of a phosphite triester linkage between an activated nucleoside phosphoramidite and a nucleoside bound to the substrate, for example, via a linker. In some instances, the nucleoside phosphoramidite is provided to the device activated. In some instances, the nucleoside phosphoramidite is provided to the device with an activator. In some instances, nucleoside phosphoramidites are provided to the device in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition of a nucleoside phosphoramidite, the device is optionally washed. In some instances, the coupling step is repeated one or more additional times, optionally with a wash step between nucleoside phosphoramidite additions to the substrate. In some instances, a polynucleotide synthesis method used herein comprises 1, 2, 3 or more sequential coupling steps. Prior to coupling, in many cases, the nucleoside bound to the device is de-protected by removal of a protecting group, where the protecting group functions to prevent polymerization. A common protecting group is 4,4′-dimethoxytrityl (DMT).

Following coupling, phosphoramidite polynucleotide synthesis methods optionally comprise a capping step. In a capping step, the growing polynucleotide is treated with a capping agent. A capping step is useful to block unreacted substrate-bound 5′-OH groups after coupling from further chain elongation, preventing the formation of polynucleotides with internal base deletions. Further, phosphoramidites activated with 1H-tetrazole may react, to a small extent, with the O6 position of guanosine. Without being bound by theory, upon oxidation with I₂/water, this side product, possibly via O6-N7 migration, may undergo depurination. The apurinic sites may end up being cleaved in the course of the final deprotection of the polynucleotide thus reducing the yield of the full-length product. The O6 modifications may be removed by treatment with the capping reagent prior to oxidation with I₂/water. In some instances, inclusion of a capping step during polynucleotide synthesis decreases the error rate as compared to synthesis without capping. As an example, the capping step comprises treating the substrate-bound polynucleotide with a mixture of acetic anhydride and 1-methylimidazole. Following a capping step, the device is optionally washed.

In some instances, following addition of a nucleoside phosphoramidite, and optionally after capping and one or more wash steps, the device bound growing nucleic acid is oxidized. The oxidation step comprises the phosphite triester is oxidized into a tetracoordinated phosphate triester, a protected precursor of the naturally occurring phosphate diester internucleoside linkage. In some instances, oxidation of the growing polynucleotide is achieved by treatment with iodine and water, optionally in the presence of a weak base (e.g., pyridine, lutidine, collidine). Oxidation may be carried out under anhydrous conditions using, e.g. tert-Butyl hydroperoxide or (1S)-(+)-(10-camphorsulfonyl)-oxaziridine (CSO). In some methods, a capping step is performed following oxidation. A second capping step allows for device drying, as residual water from oxidation that may persist can inhibit subsequent coupling. Following oxidation, the device and growing polynucleotide is optionally washed. In some instances, the step of oxidation is substituted with a sulfurization step to obtain polynucleotide phosphorothioates, wherein any capping steps can be performed after the sulfurization. Many reagents are capable of the efficient sulfur transfer, including but not limited to 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

In order for a subsequent cycle of nucleoside incorporation to occur through coupling, the protected 5′ end of the device bound growing polynucleotide is removed so that the primary hydroxyl group is reactive with a next nucleoside phosphoramidite. In some instances, the protecting group is DMT and deblocking occurs with trichloroacetic acid in dichloromethane. Conducting detritylation for an extended time or with stronger than recommended solutions of acids may lead to increased depurination of solid support-bound polynucleotide and thus reduces the yield of the desired full-length product. Methods and compositions of the disclosure described herein provide for controlled deblocking conditions limiting undesired depurination reactions. In some instances, the device bound polynucleotide is washed after deblocking. In some instances, efficient washing after deblocking contributes to synthesized polynucleotides having a low error rate.

Methods for the synthesis of polynucleotides typically involve an iterating sequence of the following steps: application of a protected monomer to an actively functionalized surface (e.g., locus) to link with either the activated surface, a linker or with a previously deprotected monomer; deprotection of the applied monomer so that it is reactive with a subsequently applied protected monomer; and application of another protected monomer for linking. One or more intermediate steps include oxidation or sulfurization. In some instances, one or more wash steps precede or follow one or all of the steps.

Methods for phosphoramidite-based polynucleotide synthesis comprise a series of chemical steps. In some instances, one or more steps of a synthesis method involve reagent cycling, where one or more steps of the method comprise application to the device of a reagent useful for the step. For example, reagents are cycled by a series of liquid deposition and vacuum drying steps. For substrates comprising three-dimensional features such as wells, microwells, channels and the like, reagents are optionally passed through one or more regions of the device via the wells and/or channels.

Methods and systems described herein relate to polynucleotide synthesis devices for the synthesis of polynucleotides. The synthesis may be in parallel. For example at least or about at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 100, 150, 200, 250, 300, 350, 400, 450, 500, 550, 600, 650, 700, 750, 800, 850, 900, 1000, 10000, 50000, 75000, 100000 or more polynucleotides can be synthesized in parallel. The total number polynucleotides that may be synthesized in parallel may be from 2-100000, 3-50000, 4-10000, 5-1000, 6-900, 7-850, 8-800, 9-750, 10-700, 11-650, 12-600, 13-550, 14-500, 15-450, 16-400, 17-350, 18-300, 19-250, 20-200, 21-150, 22-100, 23-50, 24-45, 25-40, 30-35. Those of skill in the art appreciate that the total number of polynucleotides synthesized in parallel may fall within any range bound by any of these values, for example 25-100. The total number of polynucleotides synthesized in parallel may fall within any range defined by any of the values serving as endpoints of the range. Total molar mass of polynucleotides synthesized within the device or the molar mass of each of the polynucleotides may be at least or at least about 10, 20, 30, 40, 50, 100, 250, 500, 750, 1000, 2000, 3000, 4000, 5000, 6000, 7000, 8000, 9000, 10000, 25000, 50000, 75000, 100000 picomoles, or more. The length of each of the polynucleotides or average length of the polynucleotides within the device may be at least or about at least 10, 15, 20, 25, 30, 35, 40, 45, 50, 100, 150, 200, 300, 400, 500 nucleotides, or more. The length of each of the polynucleotides or average length of the polynucleotides within the device may be at most or about at most 500, 400, 300, 200, 150, 100, 50, 45, 35, 30, 25, 20, 19, 18, 17, 16, 15, 14, 13, 12, 11, 10 nucleotides, or less. The length of each of the polynucleotides or average length of the polynucleotides within the device may fall from 10-500, 9-400, 11-300, 12-200, 13-150, 14-100, 15-50, 16-45, 17-40, 18-35, 19-25. Those of skill in the art appreciate that the length of each of the polynucleotides or average length of the polynucleotides within the device may fall within any range bound by any of these values, for example 100-300. The length of each of the polynucleotides or average length of the polynucleotides within the device may fall within any range defined by any of the values serving as endpoints of the range.

Methods for polynucleotide synthesis on a surface provided herein allow for synthesis at a fast rate. As an example, at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 100, 125, 150, 175, 200 nucleotides per hour, or more are synthesized. Nucleotides include adenine, guanine, thymine, cytosine, uridine building blocks, or analogs/modified versions thereof. In some instances, libraries of polynucleotides are synthesized in parallel on substrate. For example, a device comprising about or at least about 100; 1,000; 10,000; 30,000; 75,000; 100,000; 1,000,000; 2,000,000; 3,000,000; 4,000,000; or 5,000,000 resolved loci is able to support the synthesis of at least the same number of distinct polynucleotides, wherein polynucleotide encoding a distinct sequence is synthesized on a resolved locus. In some instances, a library of polynucleotides are synthesized on a device with low error rates described herein in less than about three months, two months, one month, three weeks, 15, 14, 13, 12, 11, 10, 9, 8, 7, 6, 5, 4, 3, 2 days, 24 hours or less. In some instances, larger nucleic acids assembled from a polynucleotide library synthesized with low error rate using the substrates and methods described herein are prepared in less than about three months, two months, one month, three weeks, 15, 14, 13, 12, 11, 10, 9, 8, 7, 6, 5, 4, 3, 2 days, 24 hours or less.

In some instances, methods described herein provide for generation of a library of polynucleotides comprising variant polynucleotides differing at a plurality of codon sites. In some instances, a polynucleotide may have 1 site, 2 sites, 3 sites, 4 sites, 5 sites, 6 sites, 7 sites, 8 sites, 9 sites, 10 sites, 11 sites, 12 sites, 13 sites, 14 sites, 15 sites, 16 sites, 17 sites 18 sites, 19 sites, 20 sites, 30 sites, 40 sites, 50 sites, or more of variant codon sites.

In some instances, the one or more sites of variant codon sites may be adjacent. In some instances, the one or more sites of variant codon sites may be not be adjacent and separated by 1, 2, 3, 4, 5, 6, 7, 8, 9, 10, or more codons.

In some instances, a polynucleotide may comprise multiple sites of variant codon sites, wherein all the variant codon sites are adjacent to one another, forming a stretch of variant codon sites. In some instances, a polynucleotide may comprise multiple sites of variant codon sites, wherein none the variant codon sites are adjacent to one another. In some instances, a polynucleotide may comprise multiple sites of variant codon sites, wherein some the variant codon sites are adjacent to one another, forming a stretch of variant codon sites, and some of the variant codon sites are not adjacent to one another.

### Large Polynucleotide Libraries Having Low Error Rates

Average error rates for polynucleotides synthesized within a library using the systems and methods provided may be less than 1 in 1000, less than 1 in 1250, less than 1 in 1500, less than 1 in 2000, less than 1 in 3000 or less often. In some instances, average error rates for polynucleotides synthesized within a library using the systems and methods provided are less than 1/500, 1/600, 1/700, 1/800, 1/900, 1/1000, 1/1100, 1/1200, 1/1250, 1/1300, 1/1400, 1/1500, 1/1600, 1/1700, 1/1800, 1/1900, 1/2000, 1/3000, or less. In some instances, average error rates for polynucleotides synthesized within a library using the systems and methods provided are less than 1/1000.

In some instances, aggregate error rates for polynucleotides synthesized within a library using the systems and methods provided are less than 1/500, 1/600, 1/700, 1/800, 1/900, 1/1000, 1/1100, 1/1200, 1/1250, 1/1300, 1/1400, 1/1500, 1/1600, 1/1700, 1/1800, 1/1900, 1/2000, 1/3000, or less compared to the predetermined sequences. In some instances, aggregate error rates for polynucleotides synthesized within a library using the systems and methods provided are less than 1/500, 1/600, 1/700, 1/800, 1/900, or 1/1000. In some instances, aggregate error rates for polynucleotides synthesized within a library using the systems and methods provided are less than 1/1000.

In some instances, an error correction enzyme may be used for polynucleotides synthesized within a library using the systems and methods provided can use. In some instances, aggregate error rates for polynucleotides with error correction can be less than 1/500, 1/600, 1/700, 1/800, 1/900, 1/1000, 1/1100, 1/1200, 1/1300, 1/1400, 1/1500, 1/1600, 1/1700, 1/1800, 1/1900, 1/2000, 1/3000, or less compared to the predetermined sequences. In some instances, aggregate error rates with error correction for polynucleotides synthesized within a library using the systems and methods provided can be less than 1/500, 1/600, 1/700, 1/800, 1/900, or 1/1000. In some instances, aggregate error rates with error correction for polynucleotides synthesized within a library using the systems and methods provided can be less than 1/1000.

Error rate may limit the value of gene synthesis for the production of libraries of gene variants. With an error rate of 1/300, about 0.7% of the clones in a 1500 base pair gene will be correct. As most of the errors from polynucleotide synthesis result in frame-shift mutations, over 99% of the clones in such a library will not produce a full-length protein. Reducing the error rate by 75% would increase the fraction of clones that are correct by a factor of 40. The methods and compositions of the disclosure allow for fast de novo synthesis of large polynucleotide and gene libraries with error rates that are lower than commonly observed gene synthesis methods both due to the improved quality of synthesis and the applicability of error correction methods that are enabled in a massively parallel and time-efficient manner. Accordingly, libraries may be synthesized with base insertion, deletion, substitution, or total error rates that are under 1/300, 1/400, 1/500, 1/600, 1/700, 1/800, 1/900, 1/1000, 1/1250, 1/1500, 1/2000, 1/2500, 1/3000, 1/4000, 1/5000, 1/6000, 1/7000, 1/8000, 1/9000, 1/10000, 1/12000, 1/15000, 1/20000, 1/25000, 1/30000, 1/40000, 1/50000, 1/60000, 1/70000, 1/80000, 1/90000, 1/100000, 1/125000, 1/150000, 1/200000, 1/300000, 1/400000, 1/500000, 1/600000, 1/700000, 1/800000, 1/900000, 1/1000000, or less, across the library, or across more than 80%, 85%, 90%, 93%, 95%, 96%, 97%, 98%, 99%, 99.5%, 99.8%, 99.9%, 99.95%, 99.98%, 99.99%, or more of the library. The methods and compositions of the disclosure further relate to large synthetic polynucleotide and gene libraries with low error rates associated with at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of the polynucleotides or genes in at least a subset of the library to relate to error free sequences in comparison to a predetermined/preselected sequence. In some instances, at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of the polynucleotides or genes in an isolated volume within the library have the same sequence. In some instances, at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of any polynucleotides or genes related with more than 95%, 96%, 97%, 98%, 99%, 99.5%, 99.6%, 99.7%, 99.8%, 99.9% or more similarity or identity have the same sequence. In some instances, the error rate related to a specified locus on a polynucleotide or gene is optimized. Thus, a given locus or a plurality of selected loci of one or more polynucleotides or genes as part of a large library may each have an error rate that is less than 1/300, 1/400, 1/500, 1/600, 1/700, 1/800, 1/900, 1/1000, 1/1250, 1/1500, 1/2000, 1/2500, 1/3000, 1/4000, 1/5000, 1/6000, 1/7000, 1/8000, 1/9000, 1/10000, 1/12000, 1/15000, 1/20000, 1/25000, 1/30000, 1/40000, 1/50000, 1/60000, 1/70000, 1/80000, 1/90000, 1/100000, 1/125000, 1/150000, 1/200000, 1/300000, 1/400000, 1/500000, 1/600000, 1/700000, 1/800000, 1/900000, 1/1000000, or less. In various instances, such error optimized loci may comprise at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900, 1000, 1500, 2000, 2500, 3000, 4000, 5000, 6000, 7000, 8000, 9000, 10000, 30000, 50000, 75000, 100000, 500000, 1000000, 2000000, 3000000 or more loci. The error optimized loci may be distributed to at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900, 1000, 1500, 2000, 2500, 3000, 4000, 5000, 6000, 7000, 8000, 9000, 10000, 30000, 75000, 100000, 500000, 1000000, 2000000, 3000000 or more polynucleotides or genes.

The error rates can be achieved with or without error correction. The error rates can be achieved across the library, or across more than 80%, 85%, 90%, 93%, 95%, 96%, 97%, 98%, 99%, 99.5%, 99.8%, 99.9%, 99.95%, 99.98%, 99.99%, or more of the library.

### Computer Systems

Any of the systems described herein, may be operably linked to a computer and may be automated through a computer either locally or remotely. In various instances, the methods and systems of the disclosure may further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the disclosure. The computer systems may be programmed to interface between the user specified base sequence and the position of a material deposition device to deliver the correct reagents to specified regions of the substrate.

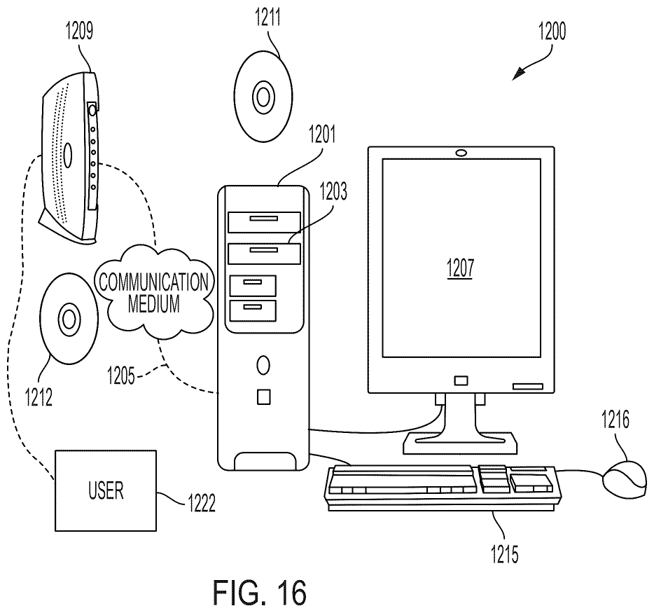

The computer system 1200 illustrated in FIG. 16 may be understood as a logical apparatus that can read instructions from media 1211 and/or a network port 1205, which can optionally be connected to server 1209 having fixed media 1212. The system, such as shown in FIG. 16 can include a CPU 1201, disk drives 1203, optional input devices such as keyboard 1215 and/or mouse 1216 and optional monitor 1207. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 1222 as illustrated in FIG. 16.

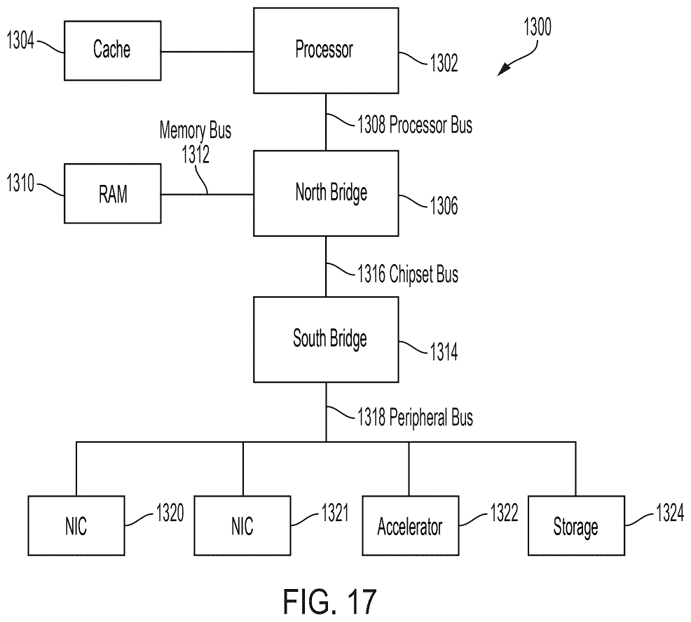

FIG. 17 is a block diagram illustrating a first example architecture of a computer system 1300 that can be used in connection with example instances of the present disclosure. As depicted in FIG. 17, the example computer system can include a processor 1302 for processing instructions. Non-limiting examples of processors include: Intel Xeon™ processor, AMD Opteron™ processor, Samsung 32-bit RISC ARM 1176JZ (F)-S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices.

As illustrated in FIG. 17, a high speed cache 1304 can be connected to, or incorporated in, the processor 1302 to provide a high speed memory for instructions or data that have been recently, or are frequently, used by processor 1302. The processor 1302 is connected to a north bridge 1306 by a processor bus 1308. The north bridge 1306 is connected to random access memory (RAM) 1310 by a memory bus 1312 and manages access to the RAM 1310 by the processor 1302. The north bridge 1306 is also connected to a south bridge 1314 by a chipset bus 1316. The south bridge 1314 is, in turn, connected to a peripheral bus 1318. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 1318. In some alternative architectures, the functionality of the north bridge can be incorporated into the processor instead of using a separate north bridge chip. In some instances, system 1300 can include an accelerator card 1322 attached to the peripheral bus 1318. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 1324 and can be loaded into RAM 1310 and/or cache 1304 for use by the processor. The system 1300 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example instances of the present disclosure. In this example, system 1300 also includes network interface cards (NICs) 1320 and 1321 connected to the peripheral bus for providing network interfaces to external storage, such as Network Attached Storage (NAS) and other computer systems that can be used for distributed parallel processing.

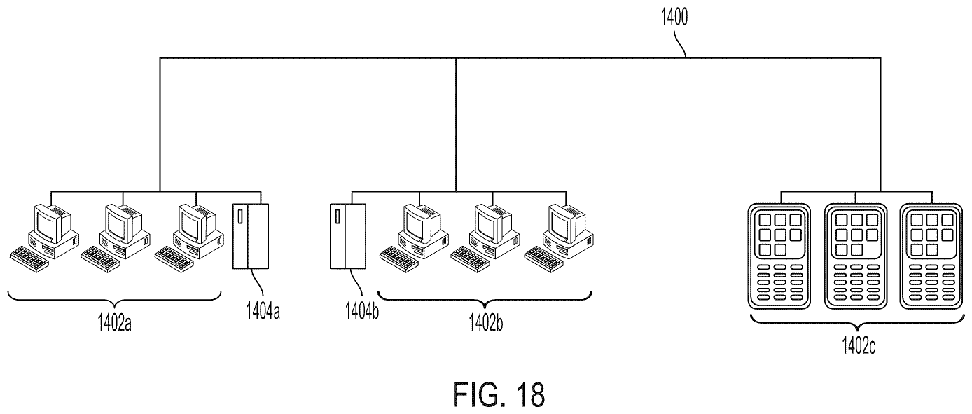

FIG. 18 is a diagram showing a network 1400 with a plurality of computer systems 1402a, and 1402b, a plurality of cell phones and personal data assistants 1402c, and Network Attached Storage (NAS) 1404a, and 1404b. In example instances, systems 1402a, 1402b, and 1402c can manage data storage and optimize data access for data stored in Network Attached Storage (NAS) 1404a and 1404b. A mathematical model can be used for the data and be evaluated using distributed parallel processing across computer systems 1402a, and 1402b, and cell phone and personal data assistant systems 1402c. Computer systems 1402a, and 1402b, and cell phone and personal data assistant systems 1402c can also provide parallel processing for adaptive data restructuring of the data stored in Network Attached Storage (NAS) 1404a and 1404b. FIG. 18 illustrates an example only, and a wide variety of other computer architectures and systems can be used in conjunction with the various instances of the present disclosure. For example, a blade server can be used to provide parallel processing. Processor blades can be connected through a back plane to provide parallel processing. Storage can also be connected to the back plane or as Network Attached Storage (NAS) through a separate network interface. In some example instances, processors can maintain separate memory spaces and transmit data through network interfaces, back plane or other connectors for parallel processing by other processors. In other instances, some or all of the processors can use a shared virtual address memory space.

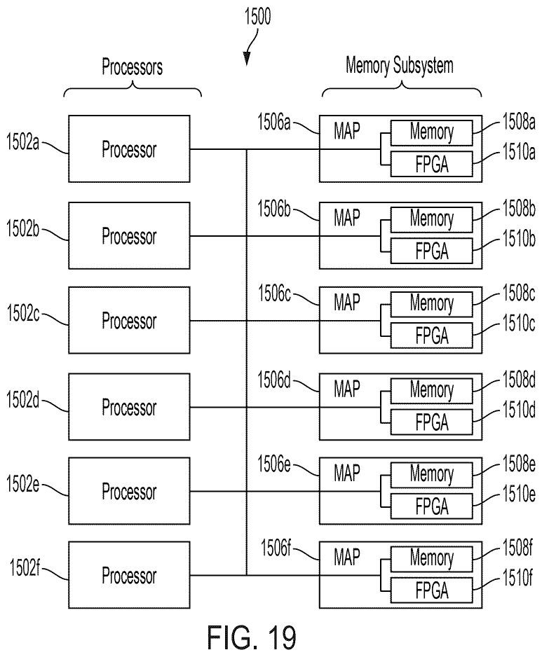

FIG. 19 is a block diagram of a multiprocessor computer system 1500 using a shared virtual address memory space in accordance with an example instance. The system includes a plurality of processors 1502a-f that can access a shared memory subsystem 1504. The system incorporates a plurality of programmable hardware memory algorithm processors (MAPs) 1506a-f in the memory subsystem 1504. Each MAP 1506a-f can comprise a memory 1508a-f and one or more field programmable gate arrays (FPGAs) 1510a-f. The MAP provides a configurable functional unit and particular algorithms or portions of algorithms can be provided to the FPGAs 1510a-f for processing in close coordination with a respective processor. For example, the MAPs can be used to evaluate algebraic expressions regarding the data model and to perform adaptive data restructuring in example instances. In this example, each MAP is globally accessible by all of the processors for these purposes. In one configuration, each MAP can use Direct Memory Access (DMA) to access an associated memory 1508a-f, allowing it to execute tasks independently of, and asynchronously from the respective microprocessor 1502a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example instances, including systems using any combination of general processors, co-processors, FPGAs and other programmable logic devices, system on chips (SOCs), application specific integrated circuits (ASICs), and other processing and logic elements. In some instances, all or part of the computer system can be implemented in software or hardware. Any variety of data storage media can be used in connection with example instances, including random access memory, hard drives, flash memory, tape drives, disk arrays, Network Attached Storage (NAS) and other local or distributed data storage devices and systems.

In example instances, the computer system can be implemented using software modules executing on any of the above or other computer architectures and systems. In other instances, the functions of the system can be implemented partially or completely in firmware, programmable logic devices such as field programmable gate arrays (FPGAs) as referenced in FIG. 19, system on chips (SOCs), application specific integrated circuits (ASICs), or other processing and logic elements. For example, the Set Processor and Optimizer can be implemented with hardware acceleration through the use of a hardware accelerator card, such as accelerator card 1322 illustrated in FIG. 17.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Functionalization of a Substrate Surface

A substrate was functionalized to support the attachment and synthesis of a library of polynucleotides. The substrate surface was first wet cleaned using a piranha solution comprising 90% H₂SO₄ and 10% H₂O₂ for 20 minutes. The substrate was rinsed in several beakers with DI water, held under a DI water gooseneck faucet for 5 minutes, and dried with N₂. The substrate was subsequently soaked in NH₄OH (1:100; 3 mL: 300 mL) for 5 minutes, rinsed with DI water using a handgun, soaked in three successive beakers with DI water for 1 minute each, and then rinsed again with DI water using the handgun. The substrate was then plasma cleaned by exposing the substrate surface to 02. A SAMCO PC-300 instrument was used to plasma etch O2 at 250 watts for 1 minute in downstream mode.

The cleaned substrate surface was actively functionalized with a solution comprising N-(3-triethoxysilylpropyl)-4-hydroxybutyramide using a YES-1224P vapor deposition oven system with the following parameters: 0.5 to 1 torr, 60 minutes, 70° C., 135° C. vaporizer. The substrate surface was resist coated using a Brewer Science 200× spin coater. SPR™ 3612 photoresist was spin coated on the substrate at 2500 rpm for 40 seconds. The substrate was pre-baked for 30 minutes at 90° C. on a Brewer hot plate. The substrate was subjected to photolithography using a Karl Suss MA6 mask aligner instrument. The substrate was exposed for 2.2 seconds and developed for 1 minute in MSF 26A. Remaining developer was rinsed with the handgun and the substrate soaked in water for 5 minutes. The substrate was baked for 30 minutes at 100° C. in the oven, followed by visual inspection for lithography defects using a Nikon L200. A descum process was used to remove residual resist using the SAMCO PC-300 instrument to O₂ plasma etch at 250 watts for 1 minute.

The substrate surface was passively functionalized with a 100 μL solution of perfluorooctyltrichlorosilane mixed with 10 μL light mineral oil. The substrate was placed in a chamber, pumped for 10 minutes, and then the valve was closed to the pump and left to stand for 10 minutes. The chamber was vented to air. The substrate was resist stripped by performing two soaks for 5 minutes in 500 mL NMP at 70° C. with ultrasonication at maximum power (9 on Crest system). The substrate was then soaked for 5 minutes in 500 mL isopropanol at room temperature with ultrasonication at maximum power. The substrate was dipped in 300 mL of 200 proof ethanol and blown dry with N₂. The functionalized surface was activated to serve as a support for polynucleotide synthesis.

### Example 2: Synthesis of a 50-Mer Sequence on a Polynucleotide Synthesis Device

A two-dimensional polynucleotide synthesis device was assembled into a flow cell, which was connected to a flow cell (Applied Biosystems (ABI394 DNA Synthesizer”). The polynucleotide synthesis device was uniformly functionalized with N-(3-TRIETHOXYSILYLPROPYL)-4-HYDROXYBUTYRAMIDE (Gelest), and was used to synthesize an exemplary polynucleotide of 50 bp (“50-mer polynucleotide”) having the sequence:

(SEQ ID NO: 1)
5′AGACAATCAACCATTTGGGGTGGACAGCCTTGACCTCTA
GACTTCGGCAT##TTTTTTTTTT3′,

where # denotes Thymidine-succinyl hexamide CED phosphoramidite, a cleavable linker enabling the release of polynucleotides from the surface during deprotection.

The synthesis was done using standard DNA synthesis chemistry (coupling, capping, oxidation, and deblocking) and an ABI synthesizer.

The phosphoramidite/activator combination was delivered similar to the delivery of bulk reagents through the flow cell. No drying steps were performed as the environment stays “wet” with reagent the entire time.

The flow restrictor was removed from the ABI 394 synthesizer to enable faster flow. Without flow restrictor, flow rates for amidites (0.1M in ACN), Activator, (0.25M Benzoylthiotetrazole (“BTT”; 30-3070-xx from GlenResearch) in ACN), and Ox (0.02M I₂ in 20% pyridine, 10% water, and 70% THF) were roughly ~100 μL/second, for acetonitrile (“ACN”) and capping reagents (1:1 mix of CapA and CapB, wherein CapA is acetic anhydride in THF/Pyridine and CapB is 16% 1-methylimidizole in THF), roughly ~200 μL/second, and for Deblock (3% dichloroacetic acid in toluene), roughly ~300 μL/second (compared to ~50 μL/second for all reagents with flow restrictor). The time to completely push out Oxidizer was observed, the timing for chemical flow times was adjusted accordingly and an extra ACN wash was introduced between different chemicals. After polynucleotide synthesis, the chip was deprotected in gaseous ammonia overnight at 75 psi. Five drops of water were applied to the surface to recover polynucleotides. The recovered polynucleotides were then analyzed on a BioAnalyzer small RNA chip (data not shown).

### Example 3: Synthesis of a 100-Mer Sequence on a Polynucleotide Synthesis Device

The same methods as described in Example 2 for the synthesis of the 50-mer polynucleotide were used to synthesize an exemplary 100-mer polynucleotide having the sequence:

(SEQ ID NO: 2)
5′CGGGATCCTTATCGTCATCGTCGTACAGATCCCGACCCATTTGCT
GTCCACCAGTCATGCTAGCCATACCATGATGATGATGATGATGAGAA
CCCCGCAT##TTTTTTTTTT3′,

where # denotes Thymidine-succinyl hexamide CED phosphoramidite (CLP-2244 from ChemGenes); on two different silicon chips, the first one uniformly functionalized with N-(3-TRIETHOXYSILYLPROPYL)-4-HYDROXYBUTYRAMIDE and the second one functionalized with 5/95 mix of 11-acetoxyundecyltriethoxysilane and n-decyltriethoxysilane, and the polynucleotides extracted from the surface were analyzed on a BioAnalyzer instrument (data not shown).

All ten samples from the two chips were further PCR amplified using a forward (5′ATGCGGGGTTCTCATCATC3′; SEQ ID NO: 3) and a reverse (5′CGGGATCCTTATCGTCATCG3′; SEQ ID NO: 4) primer in a 50 μL PCR mix (25 μL of NEB Q5 master mix, 2.5 μL of 10 μM forward primer, 2.5 μL of 10 μM reverse primer, 1 μL of polynucleotide extracted from the surface, and up to 50 μL of water) using the following thermal cycling program:
98 C, 30 seconds
98 C, 10 seconds; 63C, 10 seconds; 72C, 10 seconds; repeat 12 cycles
72C, 2 minutes

The PCR products were also run on a BioAnalyzer (data not shown), demonstrating sharp peaks at the 100-mer position. Next, the PCR amplified samples were cloned, and Sanger sequenced. Table 7 summarizes the results from the Sanger sequencing for samples taken from spots 1-5 from chip 1 and for samples taken from spots 6-10 from chip 2.

TABLE 7

| | | |
|---|---|---|
| Spot | Error rate | Cycle efficiency |

| | | | |
|---|---|---|---|
| 1 | 1/763 | bp | 99.87% |
| 2 | 1/824 | bp | 99.88% |
| 3 | 1/780 | bp | 99.87% |
| 4 | 1/429 | bp | 99.77% |
| 5 | 1/1525 | bp | 99.93% |
| 6 | 1/1615 | bp | 99.94% |
| 7 | 1/531 | bp | 99.81% |
| 8 | 1/1769 | bp | 99.94% |
| 9 | 1/854 | bp | 99.88% |
| 10 | 1/1451 | bp | 99.93% |

Thus, the high quality and uniformity of the synthesized polynucleotides were repeated on two chips with different surface chemistries. Overall, 89%, corresponding to 233 out of 262 of the 100-mers that were sequenced were perfect sequences with no errors.

Finally, Table 8 summarizes error characteristics for the sequences obtained from the polynucleotides samples from spots 1-10.

TABLE 8

| Sample | OSA_ | OSA_ | OSA_ | OSA_ | OSA_ | OSA_ | OSA_ | OSA_ | OSA_ | OSA_ |
|---|---|---|---|---|---|---|---|---|---|---|
| ID/Spot no. | 0046/1 | 0047/2 | 0048/3 | 0049/4 | 0050/5 | 0051/6 | 0052/7 | 0053/8 | 0054/9 | 0055/10 |
| Total | 132 | 232 | 332 | 432 | 532 | 632 | 732 | 832 | 932 | 32 |
| Sequences | | | | | | | | | | |
| Sequencing | 25 of 28 | 27 of 27 | 26 of 30 | 21 of 23 | 25 of 26 | 29 of 30 | 27 of 31 | 29 of 31 | 28 of 29 | 25 of 28 |
| Quality | | | | | | | | | | |
| Oligo | 23 of 25 | 25 of 27 | 22 of 26 | 18 of 21 | 24 of 25 | 25 of 29 | 22 of 27 | 28 of 29 | 26 of 28 | 20 of 25 |
| Quality | | | | | | | | | | |
| ROI Match | 2500 | 2698 | 2561 | 2122 | 2499 | 2666 | 2625 | 2899 | 2798 | 2348 |
| Count | | | | | | | | | | |
| ROI | 2 | 2 | 1 | 3 | 1 | 0 | 2 | 1 | 2 | 1 |
| Mutation | | | | | | | | | | |
| ROI Multi | 0 | 0 | 0 | 0 | 0 | 0 | 0 | 0 | 0 | 0 |
| Base | | | | | | | | | | |
| Deletion | | | | | | | | | | |
| ROI Small | 1 | 0 | 0 | 0 | 0 | 0 | 0 | 0 | 0 | 0 |
| Insertion | | | | | | | | | | |
| ROI Single | 0 | 0 | 0 | 0 | 0 | 0 | 0 | 0 | 0 | 0 |
| Base | | | | | | | | | | |
| Deletion | | | | | | | | | | |
| Large | 0 | 0 | 1 | 0 | 0 | 1 | 1 | 0 | 0 | 0 |
| Deletion | | | | | | | | | | |
| Count | | | | | | | | | | |
| Mutation: | 2 | 2 | 1 | 2 | 1 | 0 | 2 | 1 | 2 | 1 |
| G > A | | | | | | | | | | |
| Mutation: | 0 | 0 | 0 | 1 | 0 | 0 | 0 | 0 | 0 | 0 |
| T > C | | | | | | | | | | |
| ROI Error | 3 | 2 | 2 | 3 | 1 | 1 | 3 | 1 | 2 | 1 |
| Count | | | | | | | | | | |
| ROI Error | Err: ~1 | Err: ~1 | Err: ~1 | Err: ~1 | Err: ~1 | Err: ~1 | Err: ~1 | Err: ~1 | Err: ~1 | Err: ~1 |
| Rate | in 834 | in 1350 | in 1282 | in 708 | in 2500 | in 2667 | in 876 | in 2900 | in 1400 | in 2349 |
| ROI Minus | MP Err: | MP Err: | MP Err: | MP Err: | MP Err: | MP Err: | MP Err: | MP Err: | MP Err: | MP Err: |
| Primer | ~1 in | ~1 in | ~1 in | ~1 in | ~1 in | ~1 in | ~1 in | ~1 in | ~1 in | ~1 in |
| Error Rate | 763 | 824 | 780 | 429 | 1525 | 1615 | 531 | 1769 | 854 | 1451 |

### Example 4: Parallel Assembly of 29,040 Unique Olynucleotides

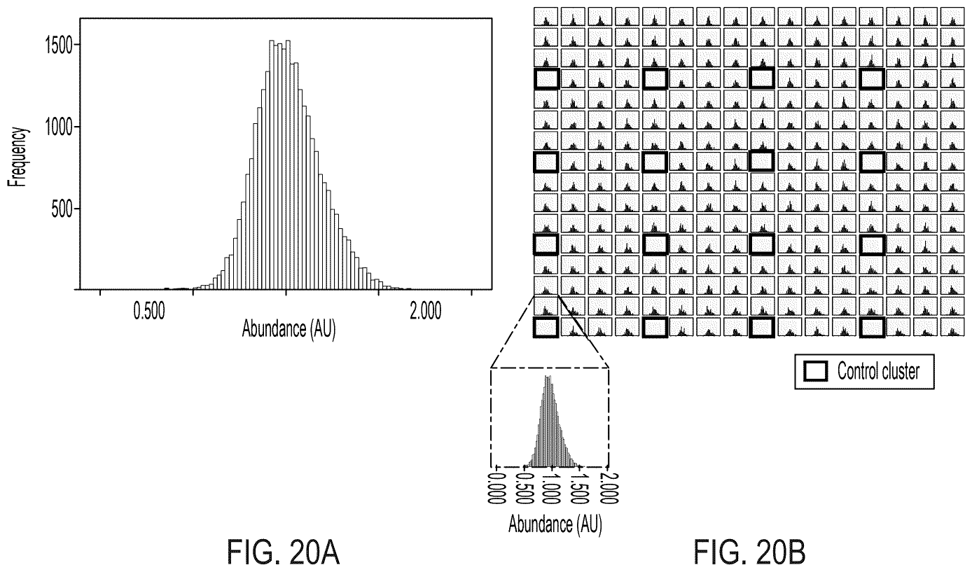

A structure comprising 256 clusters each comprising 121 loci on a flat silicon plate 201 was manufactured as shown in FIG. 15. An expanded view of a cluster is shown in 205 with 121 loci. Loci from 240 of the 256 clusters provided an attachment and support for the synthesis of polynucleotides having distinct sequences. Polynucleotide synthesis was performed by phosphoramidite chemistry using general methods from Example 3, above. Loci from 16 of the 256 clusters were control clusters. The global distribution of the 29,040 unique polynucleotides synthesized (240×121) is shown in FIG. 20A. Polynucleotide libraries were synthesized at high uniformity. 90% of sequences were present at signals within 4× of the mean, allowing for 100% representation. Distribution was measured for each cluster, as shown in FIG. 20B. On a global level, all polynucleotides in the run were present and 99% of the polynucleotides had abundance that was within 2× of the mean indicating synthesis uniformity. This same observation was consistent on a per-cluster level.

The error rate for each polynucleotide was determined using an Illumina MiSeq gene sequencer. The error rate distribution for the 29,040 unique polynucleotides averages around 1 in 500 bases, with some error rates as low as 1 in 800 bases. Distribution was measured for each cluster. The library of 29,040 unique polynucleotides was synthesized in less than 20 hours. Analysis of GC percentage versus polynucleotide representation across all of the 29,040 unique polynucleotides showed that synthesis was uniform despite GC content.

### Example 5. Design and Synthesis of a Synthetic cfDNA Variant Library

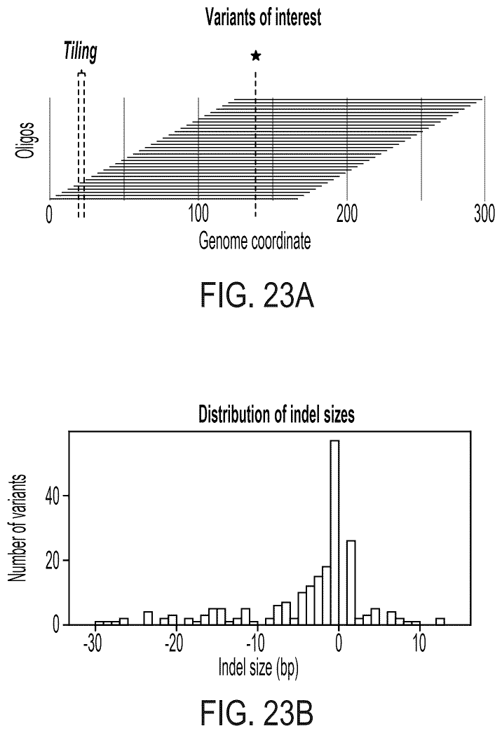

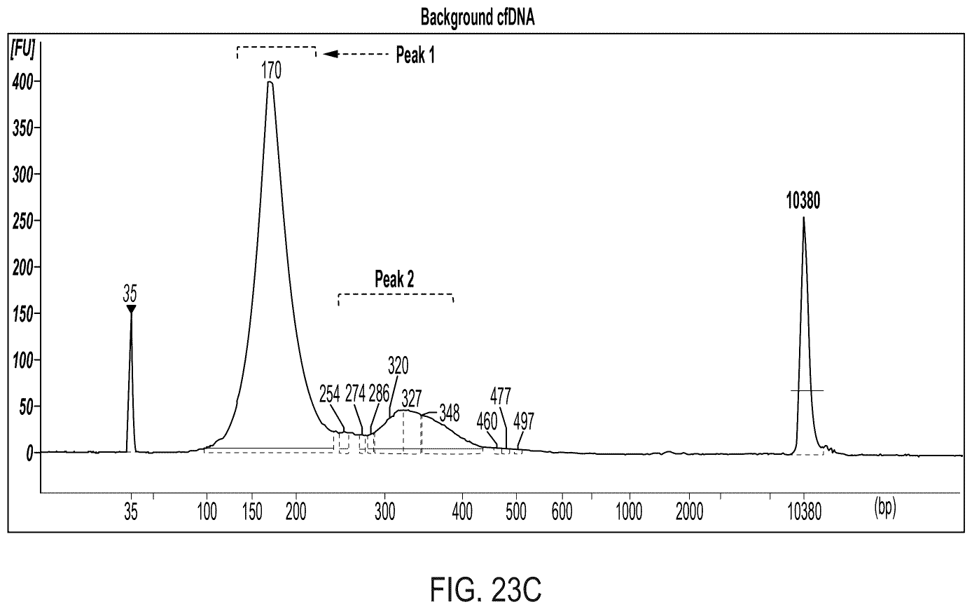

Using the general synthesis methods described in Example 3, above, a synthetic variant library was designed and synthesized. The total number of target variants represented was 458, and each polynucleotide in the library was 167 base pairs in length. Variants were present in 85 different human genes, and included SNVs (228), indels (215 total; 168 deletions, 47 insertions), fusions, and SVs (15). This included 147 clinically relevant variants (including all SVs). Variants were selected from Tables 1-6. Polynucleotides targeting a single variant were tiled using the general design of FIG. 23A, with an offset of 4 bases and with 32 polynucleotides targeting each variant. The distribution of indel sizes for the library is shown in FIG. 23B. The variant library was then mixed with a background cell-free DNA (cfDNA) library obtained from plasma of a healthy male donor (less than 30 years old, shown in FIG. 23C). Libraries having a variant allele frequency (VAF) of 0% (wild-type), 0.1%, 0.25%, 0.5%, 1%, 2%, and 5% were generated. Accurate representation and distribution of polynucleotides in the library was further confirmed by Next Generation Sequencing (all variant sites) and ddPCR (for a subset of variant sites).

### Example 6. High Sensitivity Detection of Specific Ultra-Low-Frequency Somatic Mutations for MRD Monitoring

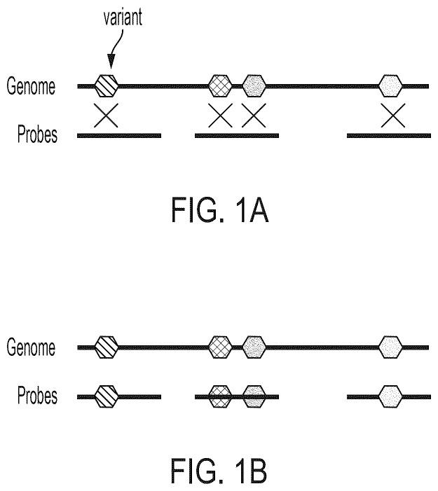

Five panels targeting minimal residual disease (MRD) were designed and developed to demonstrate the detection sensitivity. The panels specifically targeted somatic variants found in Breast, Lung, CRC, Melanoma and Renal Cell Carcinoma. Each of these MRD panels were designed to include 197 targets, with 3-5 variants per tissue origin and a selection of passenger mutations. Probe sequences of each panel were designed to incorporate the variant allele in the test sample set, as shown for example in FIG. 1B. To create the sample set, synthetic variant sequences were mixed with fragmented cell line gDNA (NA12878) to form a contrived specimen which approximates the profile cell-free and circulating tumor DNA. Five frequency levels were created with average variant allele frequencies (VAFs) of 0% (WT), 0.01%, 0.05%, 0.1% and 2%. Libraries were prepared with UMI adapters generally described herein, and target enrichment was performed using the MRD panels.

With a sequencing depth of 80,000×, variant calling results revealed that an average of 20 SNV targets can be detected with confidence in the 0.01% VAF samples for each MRD panel, clearly distinguishable from the WT control samples. In addition to demonstrating the accuracy of variant calling by targeting the alternate allele, the utility of targeting a large number of variants is showcased for the detection of an MRD signature at very low levels (e.g., 0.01% VAF). In summary, the performance of the panels showed high detection sensitivity of ultra-low-frequency somatic mutations.

### Methods

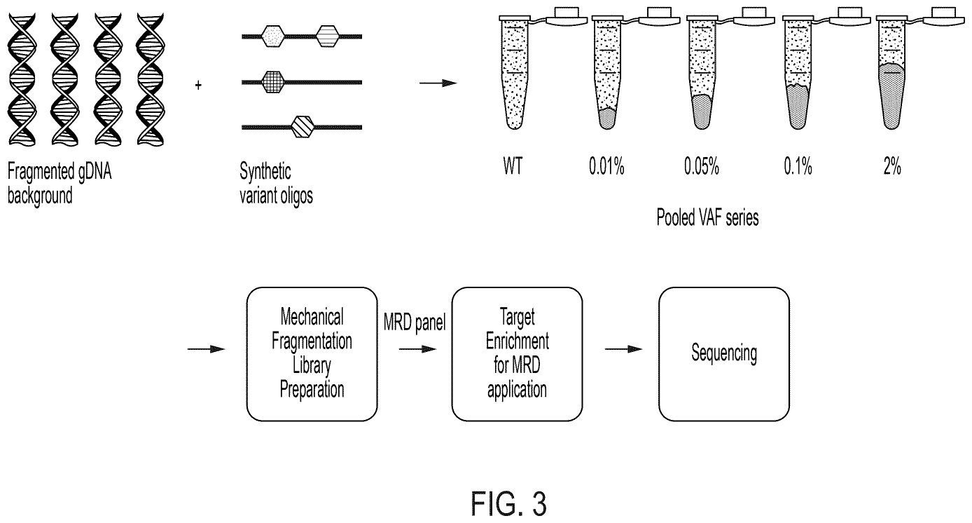

To evaluate the detection sensitivity of MRD panels, pooled VAF series were generated. The pooled VAF series were generated following the general schematic illustrated in FIG. 3. The pools comprised synthetically designed variant sequences that mimic ctDNA, combined with background gDNA that was fragmented, end-repaired, A-tailed, and purified by a Library Preparation Enzymatic Fragmentation (EF) Kit and closely mimics the DNA size profile of naive cfDNA. The ctDNA sequences were designed as a tiled pool of ~167 bp sequences that closely mimic natural ctDNA and cover 458 individual mutations including single-base substitution, small (2-4 bp), medium (5-9 bp) and large (10+ bp) insertions and deletions. Samples of 5 contrived VAF levels (0% (WT), 0.01%, 0.05%, 0.1%, 2%) were created and QCed by ddPCR, then library prepared with a Library Preparation Mechanical Fragmentation (MF) kit and UMI. These UMI libraries were captured with the five panels (targeted both reference and alternative alleles) using a standard hybridization protocol for MRD application. The MRD target enrichment libraries were sequenced using illumina Nextseq and analyzed using a UMI pipeline.

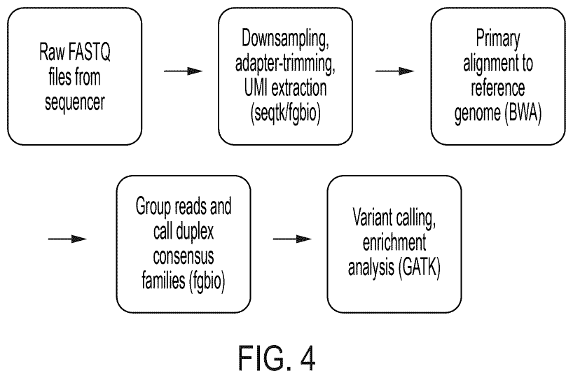

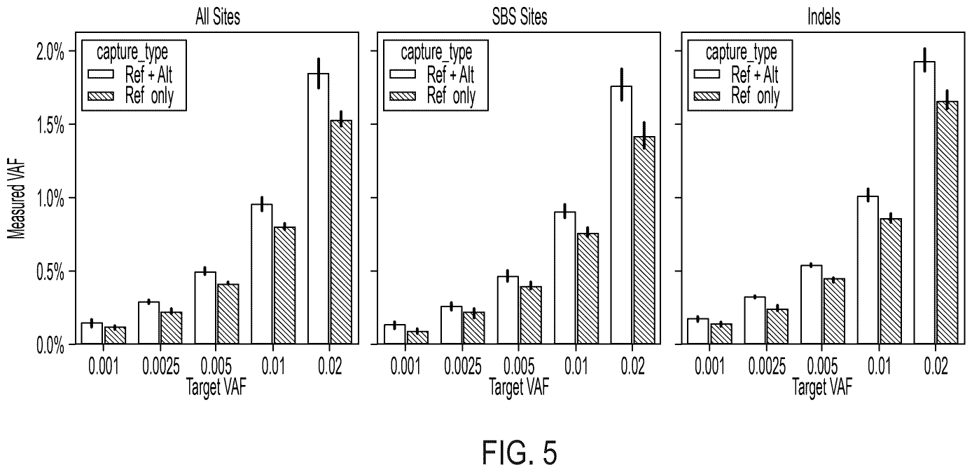

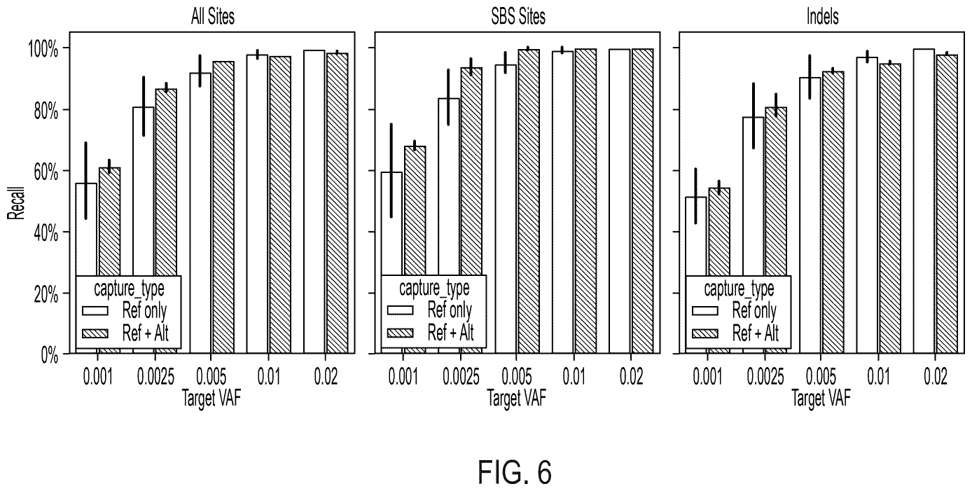

The bioinformatics workflow of the sequenced results followed the general schematic illustrated in FIG. 4. After base calling and FASTQ generation, reads were first downsampled to a fixed depth based on the target space of the panel. Reads were then pre-processed to mark adapter sequences (Picard) and to isolate UMI sequences (fgbio) into an unaligned BAM file. Raw reads were aligned to the human reference genome (hg38/GRCh38) using BWA, and were merged with the unaligned BAM to provide UMI information. After alignment, UMIs were error-corrected and grouped based on strand and UMI sequence, and consensus reads were called with a duplex strategy (fgbio). Unless otherwise specified, reads were subsequently filtered to keep only duplex consensus families, or those with at least one supporting read derived from each strand. After consensus calling, raw allele counts were obtained using samtools, or variant calls were obtained using Mutect2 (GATK) depending on the specific needs.

### Panel Performance

Five 200-probe MRD panels were designed with proprietary algorithms. These five panels specifically targeted somatic variants found in Breast, Lung, CRC, Melanoma and Renal Cell Carcinoma. To demonstrate panel performance, libraries were prepared with a mechanical library preparation kit with 30 ng of a WT cfDNA Pan-cancer Reference Standard and the UMI Adapter System for target enrichment and duplex sequencing. A standard hybridization protocol for MRD applications was also developed and optimized to further improve the MRD panel performance.

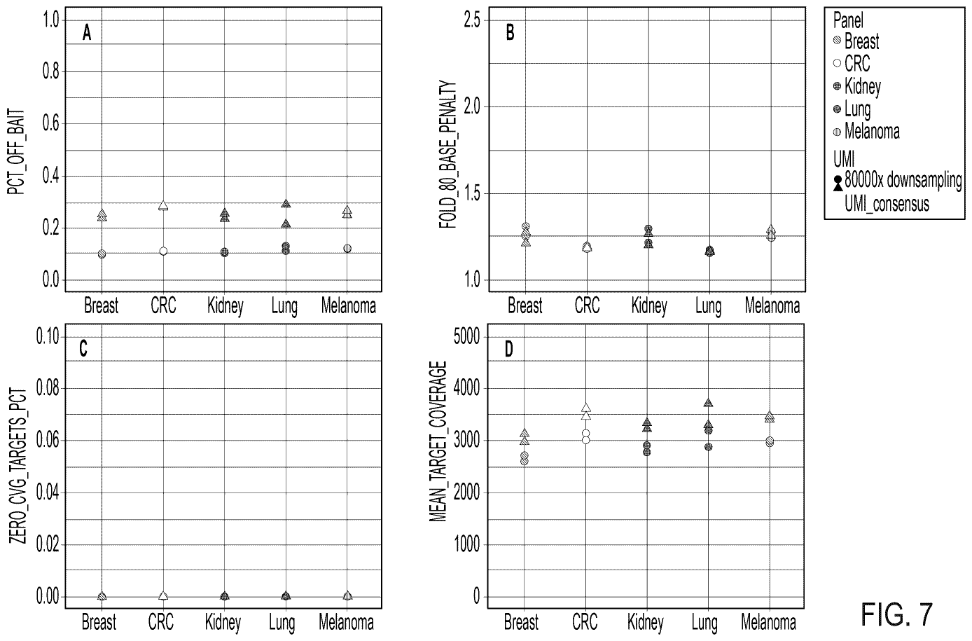

The illumina Nextseq sequencing results showed that this upgraded system was able to dramatically improve small panel performance and reduce the off-target rate in each panel to as low as 10-15% (FIG. 7A) with uniform coverage across all targets of interest (FIG. 7B). The off target rate did not greatly increase with UMI consensus analysis pipeline, suggesting the successful probe design kept specific off target rate low. In addition, no targets dropped out in MRD panels (FIG. 7C), indicating the MRD panel manufacture process and standard hybridization protocol for MRD application workflow were highly robust and accurate. With 80000× downsampling, the mean target coverage of each panel after UMI deduplication was ~3000×, generating enough coverage for variant calling analysis (FIG. 7D).

### Panel Detection Sensitivity

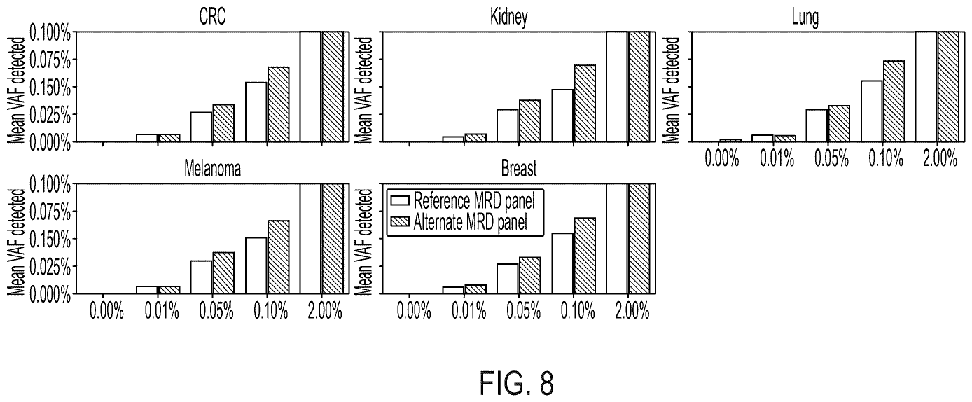

Across all panel types, variant allele frequencies approximately at 60% of the expected dilution frequency (FIG. 8) were detected, likely from a combination of capture and alignment bias. The mean error rate for the WT samples was about 10 ppm (0.001%), and the margin of error between the WT and the lowest VAF sample (0.01%) was about 7-fold, indicating that WT and 0.01% VAF samples could be cleanly distinguished. Five MRD panels targeting different somatic variants also showed very similar performance, demonstrating the consistency of the design strategy across different targets. Incorporating the variant allele rather than the reference allele into the probes improved detection sensitivity by 5-10% across different panel types at the 0.05% and 0.1% VAF levels. At 0.01% VAF level, different MRD panels showed inconsistent positive sites calling results between targeting reference allele and alternative allele, possibly due to sampling error at this low VAF level.

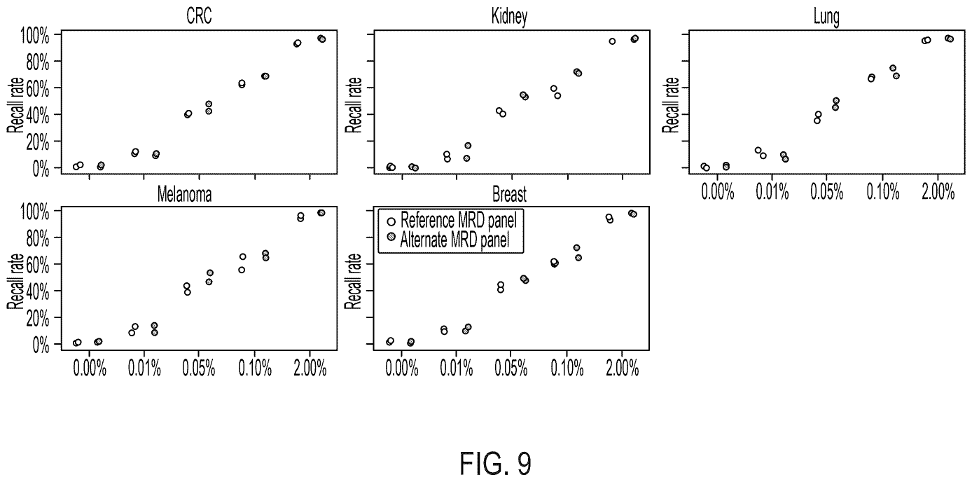

In terms of target sites recall rate (FIG. 9), almost 100% of targets had detected at 2% VAF level. While VAF impacted sensitivity, variant calling results revealed an average of at least 20/200 SNV targets detected at 0.01% VAF with 80000× sequencing depth across all panels. At 0.05%-0.1% VAF level we saw small but consistent increases in the recall rate (detection defined as at least one supporting duplex consensus read). By targeting the alternate allele in MRD panels, a ~5-10% improvement in recall for 0.05% samples was obtained, but little improvement at 0.01% likely due to sampling effects as discussed above.

### Detection Sensitivity of Indels

Insertion-deletion mutations (indels) can be important in clinical NGS, as they are implicated as drivers in many cancers. The indel detection rate can be generally affected by the mapping parameters of a short read aligner, normalization scheme for representing indel alignments, as well biases resulting from the use of targeted capture sequencing. As a result, the concordance rate for indel detection tools from short read targeted sequencing can be low.

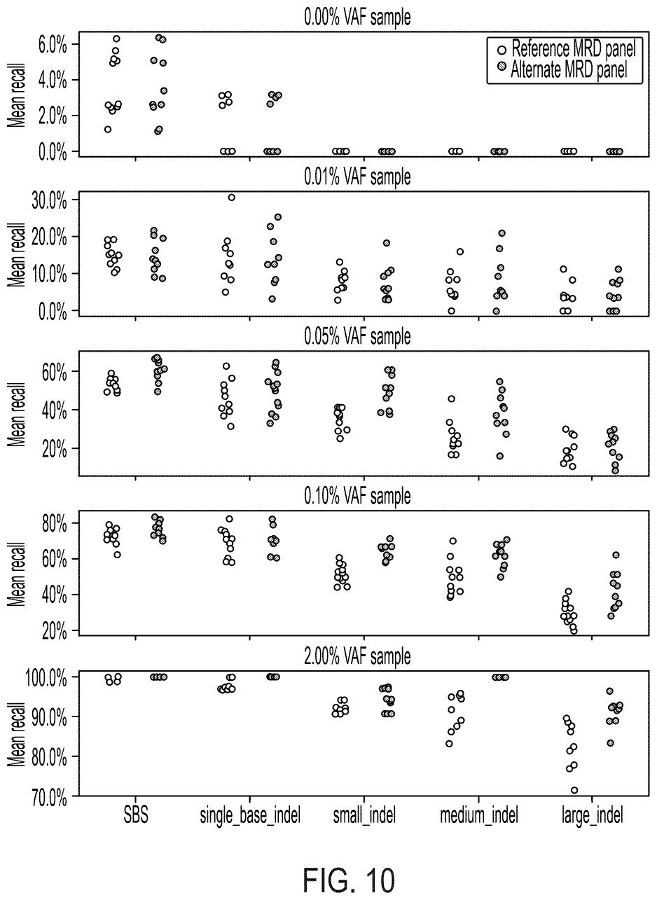

Indel detection sensitivity of MRD panels was investigated, and results showing the recall split over variant types is provided in FIG. 10. The variant types were split into SBS, single base indel, small indel (<5 bp), medium indel (5-10 bp) and large indel (>10 bp) categories. The recall rates were plotted in each VAF level over each variant type as a strip-plot, so for example, if there is 50 medium indels, detecting 20/50 in a 0.05% VAF sample would equal a recall rate of 40%. Each point in FIG. 10 represents an experiment and the panels were collapsed together into conditions. The general trends that were observed included the following: few differences were observed for the 0.01% sample; the indels tended to show more dramatic improvement in the alternative panel than the SBS's; and the effect was sometimes hard to estimate (e.g., large indels are penalized so even with the alt panel improvement can be hard to judge).

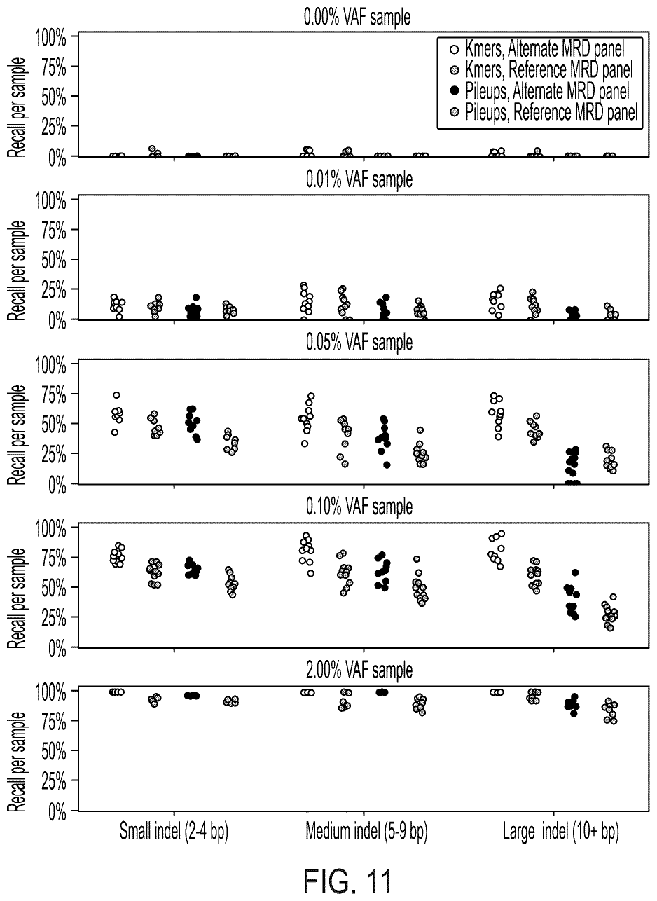

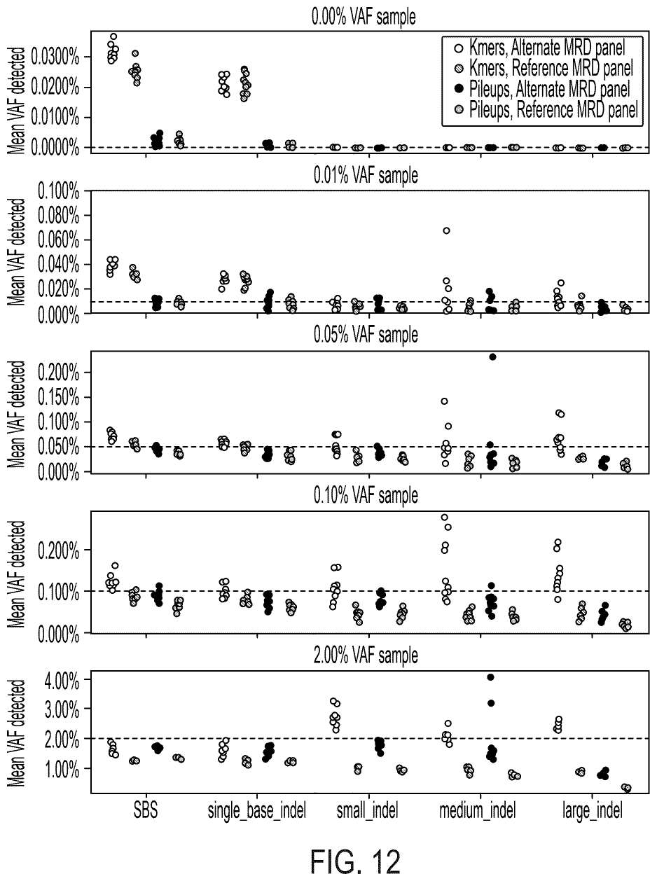

To further demonstrate the indel detection sensitivity of MRD panels, variant calling by both K-mer based searches and raw pileups from duplex-consensus read alignments were performed, and collapsed all the detected variants from each experiment into different conditions. The results showed the combination of targeting alternate alleles for enrichment and K-mer based search methods dramatically improved the detection sensitivity of larger indels (2+ bp). About 10% of total indels could be called at a 0.01% VAF level, clearly distinguishable from the WT control samples. Differences in indel recall rates tended to be most obvious for 0.05-0.1% VAF samples. At 0.1% VAF level, the recall rate could be improved from ~25% to ~75% for large events (>10 bp). In addition, medium and large indels were penalized, so even with the alternative panel the improvement was hard to predict in this range. At 2% VAF level 100% of indels were detected by targeting alternative alleles along with K-mer based search method indicating the advantage of this approach (FIG. 11). Focusing on the mean VAF detection rate at each VAF level, application of K-mer based search and targeting alternative alleles achieved consistent VAFs, which was similar to the target VAFs. Particularly in 2% VAF condition, targeting the alternate allele instead of the reference allele showed much more obvious improvement. Rates of random errors were quite high for SBS and single base indels, but low for the larger indels (FIG. 12).

### ROC Curve Analysis

ROC (Receiver Operating Characteristic) analysis was developed as a standard methodology to quantify a signal receiver's ability to correctly distinguish objects of interest from the background noise in the system. ROC curves are generally used to show the connection/trade-off between clinical sensitivity and specificity for every possible cut-off in a graphical way for a test or a combination of tests. In addition the area under the ROC curve can give an idea about the benefit of using the test(s) in question, which can provide a meaningful interpretation for disease classification from healthy subjects.

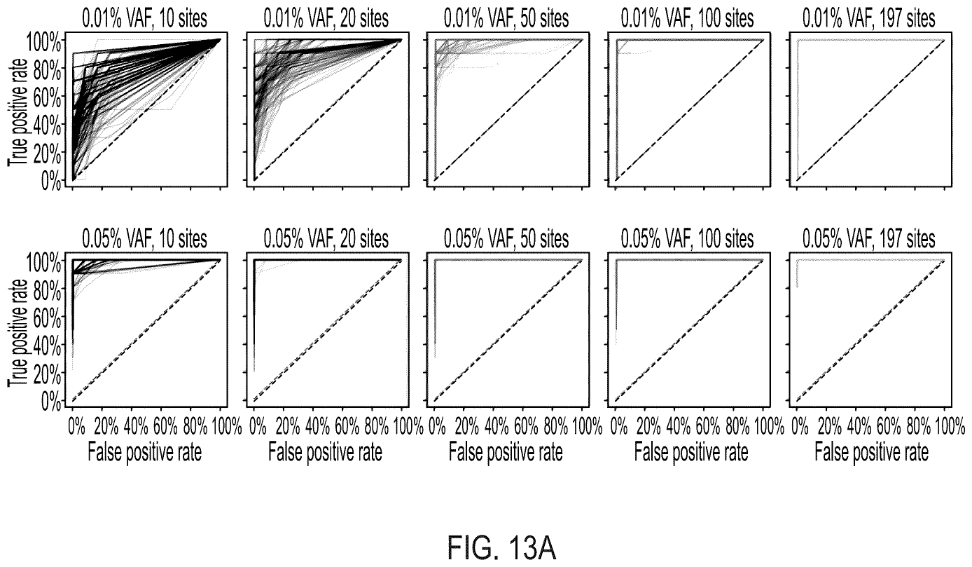

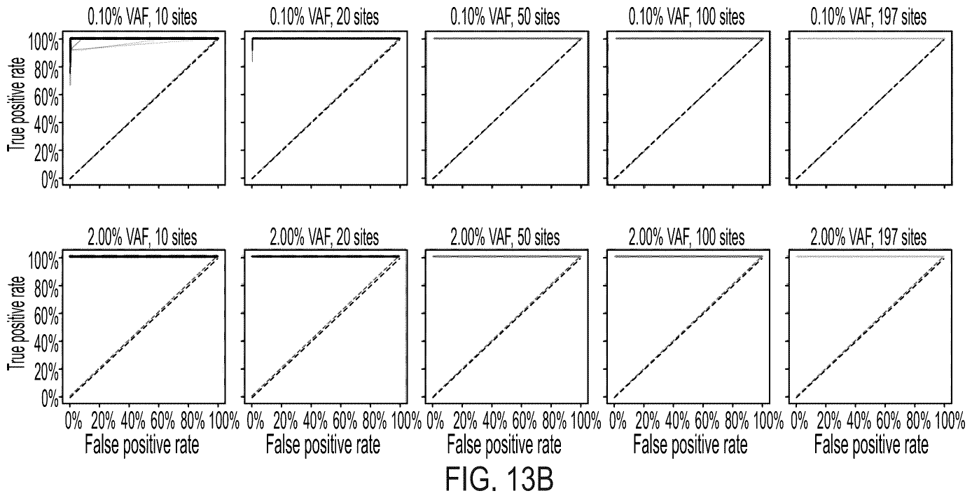

ROC analysis (FIG. 13) was applied with MRD target enrichment variant calling dataset to (1) simulate the diagnostic power of the MRD panels as compared to approaches that profile smaller numbers of sites, and (2) to find the optimal thresholds of detectable VAF levels and targeted variant sites. From the ROC analysis, larger target numbers (>50 sites) were beneficial for detection from lower VAF (<0.01%) samples. When the MRD target numbers were lower than 50 sites at 0.01% VAF samples, the simulated ROC curves were close to the diagonal line suggesting the difficulty of distinguishing between true and false positive variants. However, when MRD target numbers were higher than 50 sites at 0.01% VAF level, the MRD test was able to discriminate between true and false positive variants close to 100% sensitivity and 100% specificity. Moreover, even with target numbers as low as 10 sites, the test still showed excellent accuracy and precision at VAFs higher than 0.05%.

In a further experiment, a VAF dilution experiment using background material was performed that included an ultra-low (0.001%/10 ppm) dilution, and the results are shown in FIG. 14. Based on the results the variants were detectable against WT and served as a good proof of concept for a UMI system.

Based on the results shown, better detection sensitivity and specificity of MRD test can be achieved when incorporating more target sites (>50 sites) to the MRD panels for samples with 0.01% or lower VAF levels or obtain samples with VAF levels that equals or higher than 0.05%.

While preferred embodiments of the present subject matter have been shown and described herein, it will be obvious to those skilled in the art that such embodiments are provided by way of example only. Numerous variations, changes, and substitutions will now occur to those skilled in the art without departing from the present subject matter. It should be understood that various alternatives to the embodiments of the present subject matter described herein may be employed in practicing the present subject matter.

The present disclosure is further described by the following non-limiting items.

Item 1. A polynucleotide library comprising: a plurality of polynucleotides, wherein the plurality of polynucleotides comprises at least one variant associated with minimal residual disease (MRD).

Item 2. The library of item 1, wherein the at least one variant is within 20 bases of a center of a sequence in each of the plurality of polynucleotides.

Item 3. The library of item 1, wherein the at least one variant is within 10% of a center of a sequence in each of the plurality of polynucleotides.

Item 4. The library of item 1, wherein locations of each of the at least one variant in each sequence of the plurality of polynucleotides comprises a distribution comprising a mean.

Item 5. The library of item 4, wherein the mean is a center of each sequence.

Item 6. The library of item 4, wherein the mean is within 20 bases of the center of each sequence.

Item 7. The library of item 4, wherein the mean is within 10% of the center of each sequence.

Item 8. The library of item 4, wherein the distribution is a normal distribution.

Item 9. The library of any one of items 1-8, wherein the plurality of polynucleotides are no more than 150 bases in length.

Item 10. The library of any one of items 1-9, wherein the at least one variant is derived from genomic sequences.

Item 11. The library of any one of items 1-10, wherein the genomic sequences are derived from cell free DNA (cfDNA).

Item 12. The library of any one of items 1-11, wherein the at least one variant is present at a frequency of 0.001% to 0.1% relative to a wild-type genomic sequence.

Item 13. The library of any one of items 1-12, wherein the at least one variant comprises about 500 variants.

Item 14. The library of item 13, wherein each polynucleotide comprises one variant of the at least one variant.

Item 15. The library of any one of items 1-14, wherein the at least one variant is located in at least 150 genes.

Item 16. The library of any one of items 1-15, wherein the plurality of polynucleotides are double stranded.

Item 17. The library of any one of items 1-16, wherein the at least one variant comprises an insertion, deletion, fusion, duplication, frameshift, repeat expansion, or substitution.

Item 18. The library of any one of items 1-17, wherein the at least one variant comprises a copy number variant (CNV), microsatellite instability, loss of heterozygosity (LOH), DNA methylation, premature stop codon, trinucleotide repeat, translocation, somatic rearrangement, allelomorph, single nucleotide variant (SNV), indel, splice variant, regulator variant, copy number variant, or fusion.

Item 19. The library of any one of items 1-18, wherein the at least one variant comprises a single nucleotide variant, indel, fusion, or structural variant.

Item 20. The library of any one of items 1-19, wherein the at least one variant comprises a modification to a tumor suppressor gene or an oncogene.

Item 21. The library of any one of items 1-20, wherein the library further comprises a buffer.

Item 22. The library of any one of items 1-21, further comprising a background set comprising background polynucleotides, wherein the background set comprises cell-free DNA (cfDNA).

Item 23. The library of item 22, wherein the at least one variant comprises one or more changes compared to a background polynucleotide of the background set.

Item 24. A kit for detecting minimal residual disease (MRD) in a sample, comprising:
(a) the library of any one of items 1-23;
(b) instructions for use of the kit; and
(c) packaging configured to hold and describe the kit contents.

Item 25. The kit of item 24, wherein the kit further comprises a second library of any one of items 1-23.

Item 26. The kit of item 25, wherein the second library comprises a different frequency of the at least one variant sequence compared to the library.

Item 27. The kit of item 25 or 26, wherein the second library comprises a variant sequence that is different from the library.

Item 28. A method of preparing the library of any one of items 1-23 comprising:
(a) providing the at least one variant sequence associated with MRD; and
(b) synthesizing the plurality of polynucleotides comprising the at least one variant.

Item 29. The method of item 28, wherein the method further comprises providing the background set.

Item 30. The method of item 28 or 29, wherein the method further comprises mixing the background set and the plurality of polynucleotides comprising the at least one variant.

Item 31. The method of item 30, wherein mixing the background set and the plurality of polynucleotides comprises mixing the background set and the plurality of polynucleotides such that the at least one variant present at a frequency of 0%, 0.01%, 0.05%, 0.1%, 0.25%, 0.5%, 1%, or 2% relative to a wild-type genomic sequence.

Item 32. The method of any one of items 28-31, wherein synthesizing comprises chemical synthesis.

Item 33. The method of any one of items 28-32, wherein synthesizing comprises synthesis on a surface.

Item 34. The method of any one of items 28-33, wherein synthesizing comprises coupling of nucleoside phosphoramidites.

Item 35. The method of any one of items 28-34, wherein the method further comprises sequencing the library.

Item 36. The method of any one of items 28-35, wherein the method further comprises ddPCR measurement of the library.

Item 37. The method of any one of items 28-36, wherein the method further comprises

fluorescence/UV DNA quantification and size distribution of the library.

Item 38. A method of detecting minimal residual disease (MRD) in a sample, comprising:
(a) providing a library of any one of items 1-23;
(b) contacting the library with a sample;
(c) detecting a presence or an absence of the one or more variant sequences associated with MRD in the sample.

Item 39. The method of item 38, wherein detecting comprises sequencing.

Item 40. The method of item 39, wherein sequencing comprises Next Generation Sequencing.

Item 41. The method of item 39, wherein sequencing comprises sequencing by synthesis, nanopore sequencing, or SMRT sequencing.

Item 42. The method of any one of items 38-41, wherein detecting comprises ddPCR or specific hybridization to an array.

Item 43. The method of any one of items 38-42, wherein the at least one variant is present at a frequency of about 0.001% to 0.1% in the sample.

Item 44. The method of any one of items 38-43, wherein the method further comprises obtaining the sample from an individual.

Item 45. The method of item 44, wherein the individual was previously treated, is currently treated, or has received a clinical diagnosis for cancer.

Item 46. The method of any one of items 38-45, wherein the sample comprises a liquid biopsy.

Item 47. The method of any one of items 38-46, wherein the sample comprises circulating tumor DNA (ctDNA).

Item 48. The method of any one of items 38-47, wherein the sample is obtained from blood.

Item 49. The method of any one of items 38-48, wherein the sample is substantially cell-free.

Item 50. The method of any one of items 38-49, wherein the method further comprises ligating sequencing adapters to at least some polynucleotides in the test sample, the library, or both.

Item 51. The method of any one of items 38-50, wherein the method further comprises amplifying at least some polynucleotides in the sample, the library, or both.

Item 52. The method of any one of items 38-51, wherein a recall of the one or more variant sequences is at least 5% greater than a plurality of polynucleotides without the one or more variant sequences.

Item 53. The method of any one of items 38-52, wherein a recall of the one or more variant sequences is 5% to 10% greater than a plurality of polynucleotides without the one or more variant sequences.

1. A polynucleotide library comprising a plurality of polynucleotides, wherein each polynucleotide of the plurality of polynucleotides comprises a nucleic acid sequence having a center, and wherein the nucleic acid sequence of each polynucleotide comprises at least one variant sequence associated with minimal residual disease (MRD).

2. The polynucleotide library of claim 1, wherein a location of the at least one MRD-associated variant sequence is within 20 bases of the center of each nucleic acid sequence of each polynucleotide.

3. The polynucleotide library of claim 1, further comprising a distribution of locations of the at least one MRD-associated variant sequence in the nucleic acid sequences of all polynucleotides of the plurality of polynucleotides, wherein the distribution comprises a mean within 20 bases of the center of each nucleic acid sequence of each polynucleotide.

4. The polynucleotide library of claim 1, wherein the nucleic acid sequence of each polynucleotide is no more than 150 bases in length.

5. The polynucleotide library of claim 1, wherein the at least one MRD-associated variant sequence is derived from genomic sequences.

6. The polynucleotide library of claim 5, wherein the genomic sequences are derived from cell-free DNA (cfDNA).

7. The polynucleotide library of claim 1, wherein the at least one MRD-associated variant sequence is present in the plurality of polynucleotides at a frequency of 0.001% to 0.1% relative to a wild-type genomic sequence.

8. The polynucleotide library of claim 1, wherein the plurality of polynucleotides comprises about 500 variant sequences associated with MRD.

9. The polynucleotide library of claim 1, wherein the nucleic acid sequence of each polynucleotide comprises a variant sequence of the at least one MRD-associated variant sequence.

10. The polynucleotide library of claim 1, wherein the at least one MRD-associated variant is present in nucleic acid sequences of at least 150 genes.

11. The polynucleotide library of claim 1, wherein the at least one MRD-associated variant sequence comprises a modification relative to a nucleic acid sequence of a tumor suppressor gene or an oncogene.

12. The polynucleotide library of claim 1, further comprising a background set of polynucleotides, wherein the at least one MRD-associated variant sequence is at least one base pair different than a nucleic acid sequence of a polynucleotide of the background set.

13. (canceled)

14. A method of preparing a polynucleotide library comprising a plurality of polynucleotides, the method comprising:
providing at least one variant sequence associated with minimal residual disease (MRD); and
synthesizing a plurality of polynucleotides comprising the at least one MRD-associated variant sequence to produce the polynucleotide library, wherein each polynucleotide comprises a nucleic acid sequence having a center.

15. The method of claim 14, further comprising:
providing a background set of polynucleotides; and
mixing the background set and the plurality of polynucleotides such that the at least one MRD-associated variant sequence is present at a frequency of 0.001% to 0.1% relative to a wild-type genomic sequence.

16. The method of claim 14, wherein synthesizing comprises chemical synthesis.

17. The method of claim 14, wherein synthesizing comprises synthesis on a surface.

18. The method of claim 14, wherein synthesizing comprises coupling of nucleoside phosphoramidites.

19. The method of claim 14, further comprising sequencing the polynucleotide library.

20. A method of detecting minimal residual disease (MRD) in a sample, comprising:
providing the polynucleotide library of claim 1;
contacting the polynucleotide library with a sample; and
detecting a presence or an absence of the at least one variant associated with MRD in the sample.

21. The method of claim 20, wherein the at least one MRD-associated variant is present in the sample at a frequency of 0.001% to 0.1% relative to a wild-type genomic sequence.
